(19) United States
(12) Patent Application Publication
(10) Pub. No.: US 2022/0040162 A1
(43) Pub. Date: Feb. 10, 2022

(54) COMPOSITIONS COMPRISING A CRAC INHIBITOR AND A CORTICOSTEROID AND METHODS OF USE THEREOF

(71) Applicant: Rhizen Pharmaceuticals AG, Basel (CH)

(72) Inventors: Srikant Viswanadha, Hyberabad (IN); Swaroop Kumar Venkata Sat Vakkalanka, Basel (CH)

(21) Appl. No.: 17/250,864

(22) PCT Filed: Sep. 13, 2019
(86) PCT No.: PCT/IB2019/057746

§ 371 Date: Mar. 15, 2021

(30) Foreign Application Priority Data
Sep. 14, 2018 (IN) 201841034710

(51) Int. Cl. A61K 31/4439; A61K 31/573; A61K 31/56; A61K 31/58; A61P 11/06

(52) U.S. Cl. CPC A61K 31/4439; A61K 31/573; A61P 11/06; A61K 31/58; A61K 31/56

(57) ABSTRACT

The present disclosure relates to a method of treating an autoimmune, respiratory and/or inflammatory disease or condition (such as psoriasis, rheumatoid arthritis, asthma, or COPD) by administering at least one calcium release-activated calcium (CRAC) modulator (such as a CRAC inhibitor) and at least one corticosteroid.

This application claims the benefit of Indian Patent Application No. 201841034710, filed Sep. 14, 2018.

### FIELD OF THE INVENTION

The present disclosure relates to a method of treating an autoimmune, respiratory and/or inflammatory disease or condition (such as psoriasis, rheumatoid arthritis, asthma, or COPD) by administering at least one calcium release-activated calcium (CRAC) modulator (such as a CRAC inhibitor) and at least one corticosteroid.

### BACKGROUND OF THE INVENTION

Autoimmune, respiratory and inflammatory diseases such as rheumatoid arthritis (RA), psoriasis, systemic lupus erythematosus (SLE), chronic obstructive pulmonary disease (COPD) and asthma are chronic and often progressive diseases associated with a dysregulated or an overactive immune system, respectively. The causes and the drivers of these diseases remain ill-defined. They are characterized by complex cellular interactions between multiple inflammatory cells of the innate and adaptive immune system. Accordingly, the heterogeneity and complexity of the disease etiology of these conditions makes the search for new cellular targets challenging, as it is unclear who in the cellular infiltrate is a primary player of the pathology versus an “innocent” bystander. Therefore, targeting signalling molecules that are required for the activation of multiple immune cells may be the more likely route to success in combating these chronic, immune cell mediated diseases.

Rheumatoid arthritis (RA) is a progressive, systemic autoimmune disease characterized by chronic inflammation of multiple joints with associated systemic symptoms such as fatigue. This inflammation causes joint pain, stiffness and swelling, resulting in loss of joint function due to destruction of the bone and cartilage, often leading to progressive disability. Patients with RA also have an increased likelihood of developing other systemic complications such as osteoporosis, anaemia, and others affecting the lungs and skin.

RA is one of the most common forms of autoimmune disease and affects over 21 million people worldwide. Rheumatoid arthritis has a worldwide distribution with an estimated prevalence of 1 to 2%. Prevalence increases with age, approaching 5% in women over age 55. The average annual incidence in the United States is about 70 per 100,000 annually. Both incidence and prevalence of rheumatoid arthritis are two to three times greater in women than in men. Although rheumatoid arthritis may present at any age, patients most commonly are first affected in the third to sixth decades. RA is known to impact quality of life, causing not only physical problems but also significant negative impact on quality of life. and the disease also impacts on the average life expectancy, shortening it by three to seven years. After 10 years, less than 50% of patients with RA can work or function normally on a day-to-day basis. RA has also been reported to lead to economic burden on national economies due to hospital admissions, health care costs and lost productivity. RA is the cause of over nine million primary care physician visits in the UK annually, representing £833 million in lost production. It is also estimated to have cost the UK economy £5.5 billion in 2000. In the US, experts have estimated that RA costs more to business and industry than any other disease, with 500,000 hospitalisations per year and the burden of illness on the economy for arthritis (as a whole) to be estimated at $128 billion.

There are a number of treatments available to manage RA. Some address the signs and symptoms of RA, others aim to modify the course of the disease and positively impact the systemic effects of RA, such as fatigue and anaemia.

The current treatments include use of:

Biologics: These are genetically-engineered drugs that target specific cell surface markers or messenger substances in the immune system called cytokines, which are produced by cells in order to regulate other cells during an inflammatory response. An example of a specific cytokine targeted by biologics is tumour necrosis factor alpha (TNFα).

Traditional disease-modifying anti-rheumatic drugs (DMARDs): These are non-specific immunosuppressive drugs, which are intended to combat the signs and symptoms of RA as well as slowing down progressive joint destruction. These treatments are often used in combination with one another, or in combination with a biologic agent, to improve patient response

Glucocorticoids (corticosteroids): These are anti-inflammatory drugs related to cortisol—a steroid produced naturally in the body—that work by countering inflammation. However, the side-effects of glucocorticoids, which include hyperglycaemia, osteoporosis, hypertension, weight gain, cataracts, sleep problems, muscle loss, and susceptibility to infections, limits their use

Non-steroidal anti-inflammatory drugs (NSAIDs): These manage the signs and symptoms of RA, such as reducing pain, swelling, and inflammation, but do not alter the course of the disease or slow the progression of joint destruction

There are also a number of RA therapies targeting other components of the immune system. These include biologic treatments targeting alternative cytokines such as interleukin-6 (IL-6) that help to reduce inflammation and the progression of RA in the joints and throughout the body.

Asthma is the most common chronic disease among children and also affects millions of adults. Some 235 million people worldwide suffer from this disease. The causes of asthma are not well understood, but effective medicines are available that can treat it, thus largely avoiding the diminished lives, disabilities and death it can bring. Unfortunately, for many people with asthma—particularly the poor—effective treatments are too costly or not available at all.

Chronic obstructive pulmonary disease (COPD) is a highly prevalent condition and a major cause of morbidity and mortality worldwide. As the disease progresses, patients with COPD may become prone to frequent exacerbations, resulting in patient anxiety, worsening health status, lung function decline, and increase in mortality rate. These episodes of worsening respiratory function lead to increases in health care utilization, hospital admissions and costs. Worse, frequent exacerbations are associated with a faster decline in lung function, thereby shortening life expectancy.

According to the recommendations of Global Initiative for Chronic Obstructive Lung Disease (GOLD), the first line therapy for COPD are long acting I3-agonists, long acting muscarinic antagonist and inhalation corticosteroids. However, these drugs reduce the symptoms and exacerbations associated with the disease rather than targeting its molecular and cellular basis. Accordingly, there is still a need for further improvement of COPD therapy.

The regulation of intracellular calcium is a key element in the transduction of signals into and within cells. Cellular responses to growth factors, neurotransmitters, hormones and a variety of other signal molecules are initiated through calcium-dependent processes. The importance of calcium ion as a second messenger is emphasised by many different mechanisms which work together to maintain calcium homeostasis. Changes in intracellular free calcium ion concentration represent the most wide-spread and important signalling event for regulating a plethora of cellular responses. A widespread route for calcium ion entry into the cell is through store-operated channels (SOCs), i.e. many cell types employ store-operated calcium ion entry as their principal pathway for calcium ion influx. This mechanism is engaged following calcium ion release from stores, where the depleted stores lead to activation of calcium release-activated calcium (CRAC) channels.

CRAC channels, a subfamily of store-operated channels, are activated by the release of calcium from intracellular stores, particularly from the endoplasmic reticulum (ER). These channels are key factors in the regulation of a wide range of cellular function, including muscle contraction, protein and fluid secretion and control over cell growth and proliferation and hence play an essential role in various diseases such as immune disorders and allergic responses. Among several biophysically distinct store-operated currents, the best characterized and most calcium ion selective one is the CRAC current. Thus, CRAC channels mediate essential functions from secretion to gene expression and cell growth and form a network essential for the activation of immune cells that establish the adaptive immune response. Recently two proteins, stromal interaction molecule (STIM1) and CRAC Modulator 1 (CRACM1 or Orai1), have been identified as the essential components that fully reconstitute and amplify CRAC currents in heterologous expression systems with a similar biophysical fingerprint. In mammals, there exist several homologs of these proteins: STIM1 and STIM2 in the endoplasmic reticulum and CRACM1, CRACM2, and CRACM3 in the plasma membrane.

CRAC currents were initially discovered in lymphocytes and mast cells, and at the same time have been characterized in various cell lines such as S2 drosophila, DT40 B cells, hepatocytes, dendritic, megakaryotic, and Madin-Darby canine kidney cells. In lymphocytes and in mast cells, activation through antigen or Fc receptors initiates the release of calcium ion from intracellular stores caused by the second messenger inositol (1,4,5)-triphosphate (Ins(1,4,5)P3), which in turn leads to calcium ion influx through CRAC channels in the plasma membrane. Store-operated Ca²⁺ currents characterized in smooth muscle, A431 epidermal cells, endothelial cells from various tissues, and prostate cancer cell lines show altered biophysical characteristics suggesting a distinct molecular origin.

For example, calcium ion influx across the cell membrane is important in lymphocyte activation and adaptive immune responses. [Ca²⁺]-oscillations triggered through stimulation of the TCR (T-cell antigen receptor) have been demonstrated to be prominent and appear to involve only a single calcium ion influx pathway, the store-operated CRAC channel. See, e.g., Lewis, “Calcium signalling mechanisms in T lymphocytes,” Ann. Rev. Immunol., 19, (2001), 497-521; Feske et al., “Ca++ calcineurin signalling in cells of the immune system,” Biochem. Biophys. Res. Commun., 311, (2003), 1117-1132; Hogan et al., “Transcriptional regulation by calcium, calcineurin, and NFAT,” Genes Dev., 17, (2003) 2205-2232.

It is well established now that intracellular calcium plays an important role in various cellular functions, and that its concentration is regulated by calcium ion influx through calcium channels on the cell membrane.

Further reference is made to the following U.S patents, U.S. Publications and International Publications: WO 2005/009954, WO 2005/009539, WO 2005/009954, WO 2006/034402, WO 2006/081389, WO 2006/081391, WO 2007/087429, WO 2007/087427, WO 2007087441, WO 200/7087442, WO 2007/087443, WO 2007/089904, WO 2007109362, WO 2007/112093, WO 2008/039520, WO 2008/063504, WO 2008/103310, WO 2009/017818, WO 2009/017819, WO 2009/017831, WO 2010/039238, WO 2010/039237, WO 2010/039236, WO 2009/089305, WO 2009/038775, US 2006/0173006, US 2007/0249051, WO 2007/121186, WO 2006/050214, WO 2007/139926, WO 2008/148108, U.S. Pat. No. 7,452,675, US 2009/023177; WO 2007/139926, U.S. Pat. Nos. 6,696,267, 6,348,480, WO 2008/106731, US 2008/0293092, WO 2010/048559, WO 2010/027875, WO 2010/025295, WO 2010/034011, WO 2010/034003, WO 2009/076454, WO 2009/035818, US 2010/0152241, US 2010/0087415, US 2009/0311720, WO 2004/078995, WO 2010/122088, WO 2010/122089, WO 2011/034962, WO 2011/036130, WO 2011/139765, WO 2011/139489, WO 2011/109551, WO 2012/170931, WO 2012/027710, WO 2012/040511, WO 2012/170951, WO 2012/079020, WO 2012/056478, WO 2013/059666, WO 2013/059677, WO 2013/092463, WO 2013/092467, WO 2013/050270, WO 2013/050341, WO 2013/164773, WO 2013/164769, WO 2013/092444, WO 2013/064468, WO 2014/043715, WO 2014/059333, WO 2014/207648, WO 2014/203217, WO 2014/108336, WO 2014/108337, WO 2015/022073, WO 2015/090580, WO 2015/054283, WO 2015/197188, WO 2016/115054, WO 2017/212414 and WO 2018/140796, all of which are incorporated herein by reference in their entirety.

Other known molecules which relate to CRAC channel modulators include, for example, CM2489, CM4620, N-(5-(6-chloro-2,2-difluorobenzo[d][1,3]dioxol-5-yl)pyrazin-2-yl)-2-fluoro-6-methylbenzamide, N-[4-[3,5-Bis(trifluoromethyl)-1H-pyrazol-1-yl]phenyl]-4-methyl-1,2,3-thiadiazole-5-carboxamide (YM-58483), 2,6-Difluoro-N-{5-[4-methyl-1-(5-methyl-thiazol-2-yl)-1,2,5,6-tetrahydro-pyridin-3-yl]-pyrazin-2-yl}-benzamid (RO2959), 2,6-Difluoro-N-(1-(4-hydroxy-2-(trifluoromethyl)benzyl)-1H-pyrazol-3-yl)benzamide (GSK-7975A), 2,6-Difluoro-N-(1-(2-phenoxybenzyl)-1H-pyrazol-3-yl)benzamide (GS K5503A) and N-(2′,5′-Dimethoxy[1,1′-biphenyl]-4-yl)-3-fluoro-4-pyridinecarboxamide (Synta 66) and have been or are currently under clinical investigation for various indications.

Further reference is made herein to WO 2011/042797, WO 2011/042798, US 2011/0105447 and US 2011/0112058, each of which is incorporated herein by reference in its entirety.

Corticosteroids are potent anti-inflammatory agents, able to decrease the number, activity and movement of inflammatory cells. Corticosteroids are commonly used to treat a wide range of chronic and acute inflammatory conditions including asthma, chronic obstructive pulmonary disease (COPD), allergic rhinitis, rheumatoid arthritis, inflammatory bowel disease and autoimmune diseases. Corticosteroids mediate their effects through the glucocorticoid receptor (GR). The binding of corticosteroids to GR induces its nuclear translocation which, in turn, affects a number of downstream pathways via DNA-binding-dependent (e.g. transactivation) and -independent (e.g. transrepression) mechanisms.

Corticosteroids for treating chronic inflammatory conditions in the lung such as asthma and COPD are currently administered through inhalation. One of the advantages of employing inhaled corticosteroids (ICS) is the possibility of delivering the drug directly at site of action, thereby limiting systemic side-effects, resulting in a more rapid clinical response and a higher therapeutic ratio. Although ICS treatment can afford important benefits, especially in asthma, it is important to minimize ICS systemic exposure which leads to the occurrence and severity of unwanted side effects that may be associated with chronic administration.

Despite currently available intervention therapies, autoimmune disorders such as RA, psoriasis and respiratory disorders such as asthma and COPD remains a disease class with a significant unmet medical need.

### SUMMARY OF INVENTION

It is an objective of the present invention to provide methods and pharmaceutical compositions having enhanced activity for the treatment of respiratory and/or inflammatory diseases and conditions. Such pharmaceutical compositions allow for treating autoimmune, respiratory and inflammatory diseases and conditions with a lesser amount of active compounds and/or allow for treating autoimmune, respiratory and inflammatory diseases and conditions in a more efficient way, thereby minimizing or obviating possibly existing adverse effects generally linked to any kind of treatment with an active compound in high doses and/or for a longer period of time.

In one aspect, the present invention provides a method of treating autoimmune, respiratory and inflammatory diseases and conditions comprising administering a combination of a CRAC modulator (e.g., a CRAC inhibitor) with at least one corticosteroid.

In one embodiment, the present invention provides a method of treating an autoimmune, respiratory and/or inflammatory disease or condition comprising administering a combination of a CRAC inhibitor with at least one corticosteroid.

The present invention also relates to a combination of medicaments, comprising a CRAC inhibitor and at least one corticosteroid, and to the use thereof for the treatment of an autoimmune, respiratory and/or inflammatory disease or condition, in particular for the treatment of asthma, rheumatoid Arthritis (RA), psoriasis and/or COPD.

The present invention also relates to a pharmaceutical composition comprising a CRAC modulator (e.g., a CRAC inhibitor) and at least one corticosteroid, and to use of such a pharmaceutical composition for treating an autoimmune, respiratory or inflammatory disease or condition, such as asthma, rheumatoid Arthritis (RA), psoriasis and COPD.

In one embodiment the present invention provides a method of treating an autoimmune, respiratory and/or inflammatory disease or condition comprising administering a CRAC inhibitor, or pharmaceutically acceptable salt thereof, and a corticosteroid, or a pharmaceutical acceptable salt thereof, and to the use thereof for the treatment of an autoimmune, respiratory and/or inflammatory disease or condition, in particular for the treatment of asthma,

Yet another embodiment the present invention provides a method of treating an autoimmune, respiratory and/or inflammatory disease or condition comprising administering (i) a CRAC modulator, wherein the CRAC modulator is a compound of formula (I)

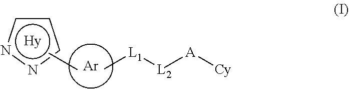

or a tautomer, N-oxide, pharmaceutically acceptable ester or pharmaceutically acceptable salt thereof, wherein

Ring Hy represents

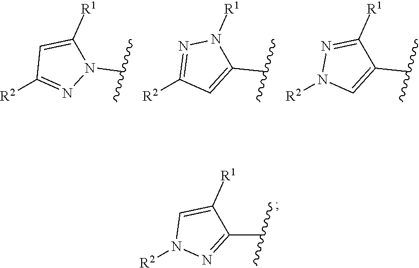

Ring Hy is optionally substituted with R′″;

R¹ and R² are the same or different and are selected from CH₃, CH₂F, CHF₂, CF₃, substituted or unsubstituted C₍₃₋₅₎cycloalkyl, CH₂—OR^{a}, CH₂—NR^{a}R^{b} and COOH;

Ring Ar represents:

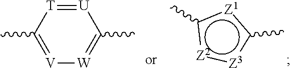

T, U, V and W are the same or different and are independently selected from CR^{a} and N;

Z¹, Z² and Z³ are the same or different and are selected from CR^{a}, CR^{a}R^{b}, O, S and —NR^{a}, with the proviso that at least one of Z¹, Z² and Z³ represents O, S or —NR^{a};

L₁ and L₂ together represent —NH—C(═X)—, —NH—S(═O)_{q}—, —C(═X)NH—, —NH—CR′R″— or —S(═O)_{q}NH—;

A is absent or selected from —(CR′R″)—, O, S(═O)_{q}, C(═X) and —NR^{a};

each occurrence of R′ and R″ are the same or different and are selected from hydrogen, hydroxy, cyano, halogen, —OR^{a}, —COOR^{a}, —S(═O)_{q}—R^{a}, —NR^{a}R^{b}, —C(═X)—R^{a}, substituted or unsubstituted C₍₁₋₆₎ alkyl group, substituted or unsubstituted C₍₁₋₆₎ alkenyl, substituted or unsubstituted C₍₁₋₆₎ alkynyl, and substituted or unsubstituted C₍₃₋₅₎ cycloalkyl, or R′ and R″ together with the common atom to which they are attached may be joined to form a saturated 3-6 member carbocyclic ring; which may optionally include one or more heteroatoms which may be same or different and are selected from O, NR^{a} and S;

R′″ is selected from hydrogen, hydroxy, cyano, halogen, —OR^{a}, —COOR^{a}, —S(═O)_{q}—R^{a}, —NR^{a}R^{b}, —C(═X)—R^{a}, substituted or unsubstituted C₍₁₋₆₎ alkyl group, substituted or unsubstituted C₍₁₋₆₎ alkenyl, substituted or unsubstituted C₍₁₋₆₎ alkynyl, and substituted or unsubstituted C₍₃₋₅₎cycloalkyl;

each occurrence of X is independently selected from O, S and —NR^{a};

Cy is selected from substituted or unsubstituted cycloalkyl group, substituted or unsubstituted heterocyclyl, substituted or unsubstituted aryl, and substituted or unsubstituted heteroaryl;

each occurrence of R^{a} and R^{b} are the same or different and are selected from hydrogen, nitro, hydroxy, cyano, halogen, —OR^{c}, —S(═O)_{q}—R^{c}, —C(═Y)—R^{c}, —CR^{c}R^{d}—C(═Y)—R^{c}, —CR^{c}R^{d}—Y—CR^{c}R^{d}—, —C(═Y)—NR^{c}R^{d}—, —NRR^{d}—C(═Y)—NR^{c}R^{d}—, —S(═O)_{q}—NR^{c}R^{d}—, —NR^{c}R^{d}—S(═O)_{q}—NR^{c}R^{d}—, —NR^{c}R^{d}—NR^{c}R^{d}—, substituted or unsubstituted alkyl, substituted or unsubstituted alkenyl, substituted or unsubstituted alkynyl, optionally substituted or unsubstituted cycloalkyl, substituted or unsubstituted cycloalkylakyl, substituted or unsubstituted cycloalkenyl, substituted or unsubstituted heterocylyl, substituted or unsubstituted heterocyclylalkyl, substituted or unsubstituted aryl, substituted or unsubstituted arylalkyl, substituted or unsubstituted heteroaryl, and substituted or unsubstituted heteroarylalkyl, or when R^{a} and R^{b} are directly bound to the same atom, they may be joined to form a substituted or unsubstituted saturated or unsaturated 3-10 member ring, which may optionally include one or more heteroatoms which may be the same or different and are selected from O, NR^{c} and S;

each occurrence of R^{c} and R^{d} may be same or different and are selected from hydrogen, nitro, hydroxy, cyano, halogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted heterocyclic group, substituted or unsubstituted heterocyclylalkyl, or when two R^{c} and/or R^{d} substituents are directly bound to the same atom, they may be joined to form a substituted or unsubstituted saturated or unsaturated 3-10 member ring, which may optionally include one or more heteroatoms which are the same or different and are selected from O, NH and S;

each occurrence of Y is independently selected from O, S and —NR^{a}; and

each occurrence of q independently represents 0, 1 or 2;

and (ii) a corticosteroid, or a pharmaceutical acceptable salt thereof. In one embodiment, the disease or condition is asthma, rheumatoid arthritis, psoriasis, or chronic obstructive pulmonary disorder (COPD).

In one preferred embodiment of any of the methods and/or compositions described herein, the CRAC modulator is a compound of formula (IA)

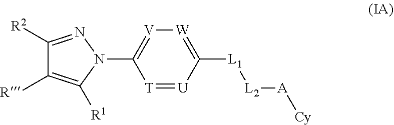

or a tautomer, N-oxide, pharmaceutically acceptable ester, or pharmaceutically acceptable salt thereof, wherein

both R¹ and R² are cyclopropyl or one of R¹ and R² is CF₃ and the other is cyclopropyl;

T is CF or N and U, V, W are independently CH, CF or N;

L₁ and L₂ together represent —NH—C(═X)—, —NH—S(═O)_{q}—, —C(═X)NH—, or —S(═O)_{q}NH— or —NH—CR′R″—;

A is absent or selected from —(CR′R″)— and —NR^{a};

each occurrence of R′ and R″ are the same or different and are independently selected from hydrogen or substituted or unsubstituted C₍₁₋₆₎ alkyl group or R′ and R″ may be joined to form a substituted or unsubstituted saturated or unsaturated 3-6 membered ring, which may optionally include one or more heteroatoms which may be same or different and are selected from O, NR^{a} and S;

R′″ is selected from hydrogen or halogen;

each occurrence of X is independently selected from O, S and —NR^{a};

Cy is selected from

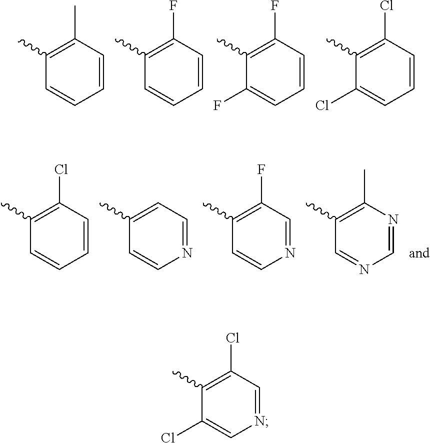

each occurrence of R^{a} is independently selected from hydrogen, nitro, hydroxy, cyano, halogen, —OR^{c}, —S(═O)_{q}—R^{c}, —NR^{c}R^{d}, —C(═Y)—R^{c}, —CR^{c}R^{d}—C(═Y)—R^{c}, —CR^{c}R^{d}—Y—CR^{c}R^{d}—, —C(═Y)—NR^{c}R^{d}—, —NRR^{d}—C(═Y)—NR^{c}R^{d}—, —S(═O)_{q}—NR^{c}R^{d}—, —NR^{c}R^{d}—S(═O)_{q}—NR^{c}R^{d}—, —NR^{c}R^{d}—NR^{c}R^{d}—, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted heterocylyl, substituted or unsubstituted heterocyclylalkyl, substituted or unsubstituted aryl, substituted or unsubstituted arylalkyl, substituted or unsubstituted heteroaryl, and substituted or unsubstituted heteroarylalkyl;

each occurrence of R^{c} and R^{d} may be same or different and are independently selected from hydrogen, nitro, hydroxy, cyano, halogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted heterocyclic group, substituted or unsubstituted heterocyclylalkyl, or when two R^{c} and/or R^{d} substitutents are directly bound to the same atom, they may be joined to form a substituted or unsubstituted saturated or unsaturated 3-10 member ring, which may optionally include one or more heteroatoms which are the same or different and are selected from O, NH and S;

each occurrence of Y is independently selected from O, S and —NR^{a}; and

each occurrence of q independently represents 0, 1 or 2.

In another preferred embodiment of any of the methods and/or compositions described herein, the CRAC modulator is a compound of formula (IB)

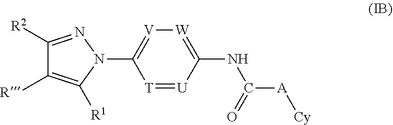

or a tautomer, N-oxide, pharmaceutically acceptable ester or pharmaceutically acceptable salt thereof, wherein

R¹ and R² are both cyclopropyl or one of R¹ and R² is CF₃ and the other is cyclopropyl;

R′″ is selected from hydrogen, hydroxy, cyano, halogen, —OR^{a}, —COOR^{a}, —S(═O)_{q}—R^{a}, —NR^{a}R^{b}, —C(═X)—R^{a}, substituted or unsubstituted C₍₁₋₆₎ alkyl group, substituted or unsubstituted C₍₁₋₆₎ alkenyl, substituted or unsubstituted C₍₁₋₆₎ alkynyl, and substituted or unsubstituted C₍₃₋₅₎cycloalkyl;

T, U, V and W are the same or different and are independently selected from CR^{a} and N;

A is absent or is selected from —CH₂—, —CHMe-,

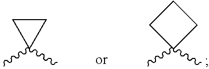

Cy is a bicyclic ring selected from substituted or unsubstituted cycloalkyl group, substituted or unsubstituted heterocyclyl, substituted or unsubstituted aryl, and substituted or unsubstituted heteroaryl;

each occurrence of R^{a} and R^{b} are the same or different and are independently selected from hydrogen, nitro, hydroxy, cyano, halogen, —OR^{c}, —S(═O)_{q}—R^{c}, —NR^{c}R^{d}, —C(═Y)—R^{c}, —CR^{c}R^{d}—C(═Y)—R^{c}, —CR^{c}R^{d}—Y—CR^{c}R^{d}—, —C(═Y)—NR^{c}R^{d}—, —NR^{c}R^{d}—C(═Y)—NR^{c}R^{d}—, —S(═O)_{q}—NR^{c}R^{d}—, —NR^{c}R^{d}—S(═O)_{q}—NR^{c}R^{d}—, —NR^{c}R^{d}—NR^{c}R^{d}—, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted heterocylyl, substituted or unsubstituted heterocyclylalkyl, substituted or unsubstituted aryl, substituted or unsubstituted arylalkyl, substituted or unsubstituted heteroaryl, and substituted or unsubstituted heteroarylalkyl, or when R^{a} and R^{b} are directly bound to the same atom, they may be joined to form a substituted or unsubstituted saturated or unsaturated 3-10 member ring, which may optionally include one or more heteroatoms which may be the same or different and are selected from O, NR^{c} and S;

each occurrence of R^{c} and R^{d} may be same or different and are independently selected from hydrogen, nitro, hydroxy, cyano, halogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted heterocyclic group, substituted or unsubstituted heterocyclylalkyl, or when two R^{c} and/or R^{d} substitutents are directly bound to the same atom, they may be joined to form a substituted or unsubstituted saturated or unsaturated 3-10 member ring, which may optionally include one or more heteroatoms which are the same or different and are selected from O, NH and S;

each occurrence of X is independently selected from O, S and —NR^{a};

each occurrence of Y is independently selected from O, S and —NR^{a}; and

each occurrence of q independently represents 0, 1 or 2.

In another preferred embodiment of any of the methods and/or compositions described herein, the CRAC modulator is a compound of formula (I), (IA) or (IB), wherein R¹ and R² are both cyclopropyl or one of R¹ and R² is CF₃ and the other is cyclopropyl.

In another preferred embodiment of any of the methods and/or compositions described herein, the CRAC modulator is a compound of formula (I), (IA) or (IB), wherein Hy is

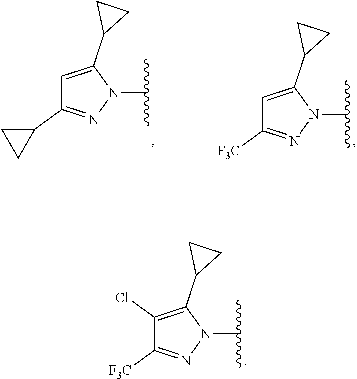 [SMILES: *n1nc(C2CC2)cc1C1CC1 ; *n1nc(C(F)(F)F)cc1C1CC1 ; *n1nc(C(F)(F)F)c(Cl)c1C1CC1]

In another preferred embodiment of any of the methods and/or compositions described herein, the CRAC modulator is a compound of formula (I), (IA) or (IB), wherein Ring Ar is selected from

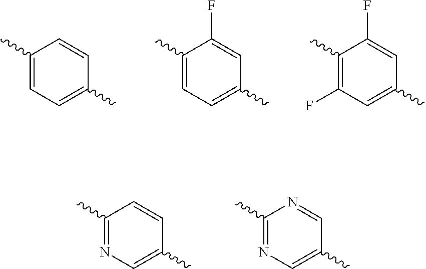

In another preferred embodiment of any of the methods and/or compositions described herein, the CRAC modulator is a compound of formula (I) and (IA) wherein L₁ and L₂ together represent —NH—C(═X)— or —C(═X)NH;

In another preferred embodiment of any of the methods and/or compositions described herein, the CRAC modulator is a compound of formula (I), (IA) or (IB), wherein Cy is selected from

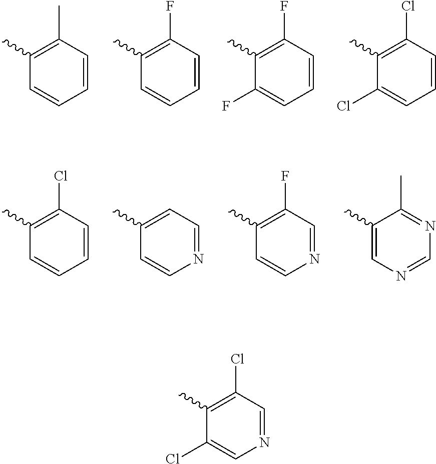

In another preferred embodiment of any of the methods and/or compositions described herein, the CRAC modulator is a compound of formula (I), (IA) or (IB), wherein Cy is selected from

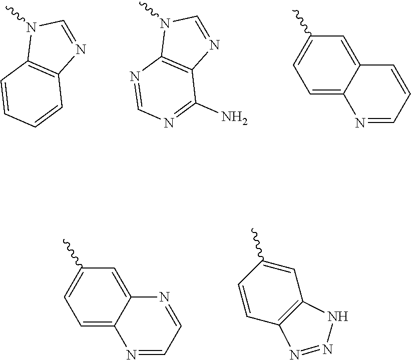

The CRAC modulators of formulas (I), (IA), and (IB) can be CRAC inhibitors.

In another preferred embodiment of any of the methods and/or compositions described herein, the CRAC modulator (e.g., CRAC inhibitor) is selected from:
N-[4-(3,5-dicyclopropyl-1H-pyrazol-1-yl)phenyl]-4-methyl-1,2,3-thiadiazole-5-carboxamide
N-[4-(3,5-dicyclopropyl-1H-pyrazol-1-yl)phenyl]-4-methylthiazole-5-carboxamide
N-[4-(3,5-dicyclopropyl-1H-pyrazol-1-yl)phenyl]-2,4-dimethylthiazole-5-carboxamide
N-[4-(3,5-dicyclopropyl-1H-pyrazol-1-yl)phenyl]-5-methylisoxazole-4-carboxamide
N-[4-(3,5-dicyclopropyl-1H-pyrazol-1-yl)phenyl]-3,5-dimethylisoxazole-4-carboxamide
N-[4-(3,5-dicyclopropyl-1H-pyrazol-1-yl)phenyl]benzamide
N-[4-(3,5-dicyclopropyl-1H-pyrazol-1-yl)phenyl]-2-methylbenzamide
N-[4-(3,5-dicyclopropyl-1H-pyrazol-1-yl)phenyl]-2,6-difluorobenzamide
N-[4-(3,5-dicyclopropyl-1H-pyrazol-1-yl)phenyl]-2,3-difluorobenzamide
N-[4-(3,5-dicyclopropyl-1H-pyrazol-1-yl)phenyl-3-(methylsulfonyl)benzamide
N-[4-(3,5-dicyclopropyl-1H-pyrazol-1-yl)phenyl]-4-(methylsulfonyl)benzamide
2-chloro-N-[4-(3,5-dicyclopropyl-1H-pyrazol-1-yl)phenyl]-5-(methylthio)benzamide
2-chloro-N-[4-(3,5-dicyclopropyl-1H-pyrazol-1-yl)phenyl)-5-(methylsulfonyl)benzamide
N-[4-(3,5-dicyclopropyl-1H-pyrazol-1-yl)phenyl]nicotinamide hydrochloride
N-[4-(3,5-dicyclopropyl-1H-pyrazol-1-yl)phenyl]isonicotinamide hydrochloride
N-[4-(3,5-dicyclopropyl-1H-pyrazol-1-yl)phenyl]-3-fluoroisonicotinamide
3,5-dichloro-N-(4-(3,5-dicyclopropyl-1H-pyrazol-1-yl)phenyl)isonicotinamide
N-[4-(3,5-dicyclopropyl-1H-pyrazol-1-yl)phenyl]-4-methylpyrimidine-5-carboxamide
N-[4-(3,5-dicyclopropyl-1H-pyrazol-1-yl)phenyl]-2-phenylacetamide
N-[4-(3,5-dicyclopropyl-1H-pyrazol-1-yl)phenyl]-2-(4-fluorophenyl)acetamide
N-[4-(3,5-dicyclopropyl-1H-pyrazol-1-yl)phenyl]-1-phenylcyclopropanecarboxamide
N-[4-(3,5-dicyclopropyl-1H-pyrazol-1-yl)phenyl]-2-(pyridin-2-yl)acetamide
N-[4-(3,5-dicyclopropyl-1H-pyrazol-1-yl)phenyl]-2-(pyridin-3-yl)acetamide
N-[4-(3,5-dicyclopropyl-1H-pyrazol-1-yl)phenyl]-2-(pyridin-4-yl)acetamide
N-[4-(3,5-dicyclopropyl-1H-pyrazol-1-yl)phenyl]-2-(piperazin-1-yl)acetamide
N-[4-(3,5-dicyclopropyl-1H-pyrazol-1-yl)phenyl]-2-morpholinoacetamide
N-[4-(3,5-dicyclopropyl-1H-pyrazol-1-yl)phenyl]benzenesulfonamide
N-[4-(3,5-dicyclopropyl-1H-pyrazol-1-yl)-3-fluorophenyl]-4-methyl-1,2,3-thiadiazole-5-carboxamide
N-[4-(3,5-dicyclopropyl-1H-pyrazol-1-yl)-3-fluorophenyl]-4-methylthiazole-5-carboxamide
N-[4-(3,5-dicyclopropyl-1H-pyrazol-1-yl)-3-fluorophenyl]-3,5-dimethylisoxazole-4-carboxamide
N-[4-(3,5-dicyclopropyl-1H-pyrazol-1-yl)-3-fluorophenyl]-2methyl benzamide
N-[4-(3,5-dicyclopropyl-1H-pyrazol-1-yl)-3-fluorophenyl]-2,3-difluorobenzamide
N-[4-(3,5-dicyclopropyl-1H-pyrazol-1-yl)-3-fluorophenyl]-2,6-difluorobenzamide
N-[4-(3,5-dicyclopropyl-1H-pyrazol-1-yl)-3-fluorophenyl]nicotinamide
N-[4-(3,5-dicyclopropyl-1H-pyrazol-1-yl)-3-fluorophenyl]isonicotinamide
N-[4-(3,5-dicyclopropyl-1H-pyrazol-1-yl)-3-fluorophenyl]-4-methylpyrimidine-5-carboxamide
N-[4-(4-chloro-3,5-dicyclopropyl-1H-pyrazol-1-yl)-3-fluorophenyl]-4-methyl-1,2,3-thiadiazole-5-carboxamide
N-[6-(3,5-dicyclopropyl-1H-pyrazol-1-yl)pyridin-3-yl]-4-methyl-1,2,3-thiadiazole-5-carboxamide
N-[6-(3,5-dicyclopropyl-1H-pyrazol-1-yl)pyridin-3-yl]-4-methylthi azole-5-carboxamide
N-[6-(3,5-dicyclopropyl-1H-pyrazol-1-yl)pyridin-3-yl]-2,4-dimethylthiazole-5-carboxamide
N-[6-(3,5-dicyclopropyl-1H-pyrazol-1-yl)pyridin-3-yl]-3,5-dimethylisoxazole-4-carboxamide
6-(3,5-dicyclopropyl-1H-pyrazol-1-yl)-N-o-tolylnicotinamide
N-[6-(3,5-dicyclopropyl-1H-pyrazol-1-yl)pyridin-3-yl]-2-fluorobenzamide
N-[6-(3,5-dicyclopropyl-1H-pyrazol-1-yl)pyridin-3-yl]-2,3-difluorobenzamide
N-[6-(3,5-dicyclopropyl-1H-pyrazol-1-yl)pyridin-3-yl]-2,6-difluorobenzamide
N-[6-(3,5-dicyclopropyl-1H-pyrazol-1-yl)pyridin-3-yl]nicotinamide dihydrochloride
N-[6-(3,5-dicyclopropyl-1H-pyrazol-1-yl)pyridin-3-yl]isonicotinamide
N-[6-(3,5-dicyclopropyl-1H-pyrazol-1-yl)pyridin-3-yl]-3-fluoroisonicotinamide
3,5-dichloro-N-{4-[5-cyclopropyl-3-(trifluoromethyl)-1H-pyrazol-1-yl]phenyl}isonicotinamide
3,5-dichloro-N-[6-(3,5-dicyclopropyl-1H-pyrazol-1-yl)pyridin-3-yl]isonicotinamide
N-[6-(3,5-dicyclopropyl-1H-pyrazol-1-yl)pyridin-3-yl]-4-methylpyrimidine-5-carboxamide
N-{4-[5-cyclopropyl-3-(trifluoromethyl)-1H-pyrazol-1-yl]phenyl}-4-methyl-1,2,3-thiadiazole-5-carboxamide
N-{4-[5-cyclopropyl-3-(trifluoromethyl)-1H-pyrazol-1-yl]phenyl}-4-methylthiazole-5-carboxamide
N-{4-[5-cyclopropyl-3-(trifluoromethyl)-1H-pyrazol-1-yl]phenyl}-N,4-dimethylthiazole-5-carboxamide
N-{4-[5-cyclopropyl-3-(trifluoromethyl)-1H-pyrazol-1-yl]phenyl}-2,4-dimethylthiazole-5-carboxamide
N-{4-[5-cyclopropyl-3-(trifluoromethyl)-1H-pyrazol-1-yl]phenyl}-5-methylisoxazole-4-carboxamide
N-{4-[5-cyclopropyl-3-(trifluoromethyl)-1H-pyrazol-1-yl]phenyl}-3,5-dimethylisoxazole-4-carboxamide
N-{4-[5-cyclopropyl-3-(trifluoromethyl)-1H-pyrazol-1-yl]phenyl}-1-methyl-1H-imidazole-2-carboxamide
N-{4-[3-cyclopropyl-5-(trifluoromethyl)-1H-pyrazol-1-yl]phenyl}-4-methyl-1H-imidazole-5-carboxamide
N-{4-[5-cyclopropyl-3-(trifluoromethyl)-1H-pyrazol-1-yl]phenyl}-2-methylbenzamide
N-{4-[5-cyclopropyl-3-(trifluoromethyl)-1H-pyrazol-1-yl]phenyl}-2,3-difluorobenzamide
N-{4-[5-cyclopropyl-3-(trifluoromethyl)-1H-pyrazol-1-yl]phenyl}-2,6-difluorobenzamide
N-{4-[5-cyclopropyl-3-(trifluoromethyl)-1H-pyrazol-1-yl]phenyl}-3-(methylsulfonyl)benzamide
2-chloro-N-{4-[5-cyclopropyl-3-(trifluoromethyl)-1H-pyrazol-1-yl]phenyl}-5-(methylthio) benzamide
2-chloro-N-{4-[5-cyclopropyl-3-(trifluoromethyl)-1H-pyrazol-1-yl]phenyl}-5-(methylsulfonyl)benzamide
N-{4-[5-cyclopropyl-3-(trifluoromethyl)-1H-pyrazol-1-yl]phenyl}pyridine-4-carboxamide hydrochloride
N-{4-[5-cyclopropyl-3-(trifluoromethyl)-1H-pyrazol-1-yl]phenyl}-3-fluoro isonicotinamide
N-{4-[5-cyclopropyl-3-(trifluoromethyl)-1H-pyrazol-1-yl]phenyl}-4-methylpyrimidine-5-carboxamide
N-{4-[5-cyclopropyl-3-(trifluoromethyl)-1H-pyrazol-1-yl]phenyl}-2,4-dimethyl pyrimidine-5-carboxamide
N-{4-[5-cyclopropyl-3-(trifluoromethyl)-1H-pyrazol-1-yl]phenyl}-2-(4-fluorophenyl)acetamide
N-{4-[5-cyclopropyl-3-(trifluoromethyl)-1H-pyrazol-1-yl]phenyl}-2-(pyridin-2-yl)acetamide
N-{4-[5-cyclopropyl-3-(trifluoromethyl)-1H-pyrazol-1-yl]phenyl}-2-(pyridin-3-yl)acetamide
N-{4-[5-cyclopropyl-3-(trifluoromethyl)-1H-pyrazol-1-yl]phenyl}-2-(pyridin-4-yl)acetamide
4-[5-cyclopropyl-3-(trifluoromethyl)-1H-pyrazol-1-yl]-N-[(4-methylthiazol-5-yl)methyl]aniline
1-{4-[5-cyclopropyl-3-(trifluoromethyl)-1H-pyrazol-1-yl]phenyl}-3-(4-methyl-1,2,3-thiadiazol-5-yl)urea
1-{4-[5-cyclopropyl-3-(trifluoromethyl)-1H-pyrazol-1-yl]phenyl}-3-(4-methylthiazol-5-yl)urea
1-{4-[5-cyclopropyl-3-(trifluoromethyl)-1H-pyrazol-1-yl]phenyl}-3-(4-methylpyrimidin-5-yl)urea
4-[5-cyclopropyl-3-(trifluoromethyl)-1H-pyrazol-1-yl]-N-(4-methylthiazol-5-yl) benzamide
4-[5-cyclopropyl-3-(trifluoromethyl)-1H-pyrazol-1-yl]-N-(2,6-difluorophenyl) benzamide
N-{4-[4-chloro-5-cyclopropyl-3-(trifluoromethyl)-1H-pyrazol-1-yl]phenyl}-4-methylthiazole-5-carboxamide
N-{4-[4-chloro-5-cyclopropyl-3-(trifluoromethyl)-1H-pyrazol-1-yl]phenyl}-2-(pyridin-2-yl)acetamide
N-{4-[5-cyclopropyl-3-(trifluoromethyl)-1H-pyrazol-1-yl]-3-fluorophenyl}-4-methyl-1,2,3-thiadiazole-5-carboxamide
N-{4-[5-cyclopropyl-3-(trifluoromethyl)-1H-pyrazol-1-yl]-3-fluorophenyl}-4-methylthiazole-5-carboxamide
N-{4-[5-cyclopropyl-3-(trifluoromethyl)-1H-pyrazol-1-yl]-3-fluorophenyl}-5-methylisoxazole-4-carboxamide
N-{4-[5-cyclopropyl-3-(trifluoromethyl)-1H-pyrazol-1-yl]-3-fluorophenyl}-3,5-dimethylisoxazole-4-carboxamide
N-{4-[5-cyclopropyl-3-(trifluoromethyl)-1H-pyrazol-1-yl]-3-fluorophenyl}-2-methylbenzamide
N-{4-[5-cyclopropyl-3-(trifluoromethyl)-1H-pyrazol-1-yl]-3-fluorophenyl}-2,3-difluorobenzamide
N-{4-[5-cyclopropyl-3-(trifluoromethyl)-1H-pyrazol-1-yl]-3-fluorophenyl}-2,6-difluorobenzamide
N-{4-[5-cyclopropyl-3-(trifluoromethyl)-1H-pyrazol-1-yl]-3-fluorophenyl}nicotinamide
N-{4-[5-cyclopropyl-3-(trifluoromethyl)-1H-pyrazol-1-yl]-3-fluorophenyl}isonicotinamide
N-{4-[5-cyclopropyl-3-(trifluoromethyl)-1H-pyrazol-1-yl]-3-fluorophenyl}-3-fluoroisonicotinamide
3,5-dichloro-N-{4-[5-cyclopropyl-3-(trifluoromethyl)-1H-pyrazol-1-yl]-3-fluorophenyl}isonicotinamide
N-{4-[5-cyclopropyl-3-(trifluoromethyl)-1H-pyrazol-1-yl]-3-fluorophenyl}-4-methylpyrimidine-5-carboxamide
N-{4-[5-cyclopropyl-3-(trifluoromethyl)-1H-pyrazol-1-yl]-3-fluorophenyl}-N,4-dimethylpyrimidine-5-carboxamide
N-{4-[4-chloro-5-cyclopropyl-3-(trifluoromethyl)-1H-pyrazol-1-yl]-3-fluorophenyl}-4-methyl-1,2,3-thiadiazole-5-carboxamide
N-{4-[4-chloro-5-cyclopropyl-3-(trifluoromethyl)-1H-pyrazol-1-yl]-3-fluorophenyl}-2-(pyridin-2-yl)acetamide
1-{4-[5-cyclopropyl-3-(trifluoromethyl)-1H-pyrazol-1-yl]-3-fluorophenyl}-3-(4-methylpyrimidin-5-yl)urea
N-{4-[5)-cyclopropyl-3-(trifluromethyl)-1H-pyrazol-1-yl]3-fluorophenyl}-2,6-dichloro benzamide
4-[5-cyclopropyl-3-(trifluoromethyl)-1H-pyrazol-1-yl]-N-(2,3-difluorophenyl)-3-fluorobenzamide
4-[5-cyclopropyl-3-(trifluoromethyl)-1H-pyrazol-1-yl]-N-(2,6-difluorophenyl)-3-fluorobenzamide
N-{6-[5-cyclopropyl-3-(trifluoromethyl)-1H-pyrazol-1-yl]pyridin-3-yl}-4-methyl-1,2,3-thiadiazole-5-carboxamide
N-{6-[5-cyclopropyl-3-(trifluoromethyl)-1H-pyrazol-1-yl]pyridin-3-yl}-4-methylthiazole-5-carboxamide
N-{6-[5-cyclopropyl-3-(trifluoromethyl)-1H-pyrazol-1-yl]pyridin-3-yl}-3,5-dimethylisoxazole-4-carboxamide
N-{6-[5-cyclopropyl-3-(trifluoromethyl)-1H-pyrazol-1-yl]pyridin-3-yl}-2-methylbenzamide
2-chloro-N-{6-[5-cyclopropyl-3-(trifluoromethyl)-1H-pyrazol-1-yl]pyridin-3-yl}benzamide
N-(6-(5-cyclopropyl-3-(trifluoromethyl)-1H-pyrazol-1-yl)pyridin-3-yl)-2-fluorobenzamide
N-{6-[5-cyclopropyl-3-(trifluoromethyl)-1H-pyrazol-1-yl]pyridin-3-yl}-2,3-difluorobenzamide
N-{6-[5-cyclopropyl-3-(trifluoromethyl)-1H-pyrazol-1-yl]pyridin-3-yl}-2,6-difluorobenzamide
N-{6-[5-cyclopropyl-3-(trifluoromethyl)-1H-pyrazol-1-yl]pyridin-3-yl}picolinamide
N-{6-[5-cyclopropyl-3-(trifluoromethyl)-1H-pyrazol-1-yl]pyridin-3-yl}-3-methylpicolinamide
N-{6-[5-cyclopropyl-3-(trifluoromethyl)-1H-pyrazol-1-yl]pyridin-3-yl}nicotinamide
N-{6-[5-cyclopropyl-3-(trifluoromethyl)-1H-pyrazol-1-yl]pyridin-3-yl}-2-methylnicotinamide
N-{6-[5-cyclopropyl-3-(trifluoromethyl)-1H-pyrazol-1-yl]pyridin-3-yl}isonicotinamide
N-{6-[5-cyclopropyl-3-(trifluoromethyl)-1H-pyrazol-1-yl]pyridin-3-yl}-3-fluoroisonicotinamide
3,5-dichloro-N-{6-[5-cyclopropyl-3-(trifluoromethyl)-1H-pyrazol-1-yl]pyridin-3-yl}isonicotinamide
N-{6-[5-cyclopropyl-3-(trifluoromethyl)-1H-pyrazol-1-yl]pyridin-3-yl}-4-methylpyrimidine-5-carboxamide
N-{6-[5-cyclopropyl-3-(trifluoromethyl)-1H-pyrazol-1-yl]pyridin-3-yl}-2-(pyridin-2-yl)acetamide
N-{6-[5-cyclopropyl-3-(trifluoromethyl)-1H-pyrazol-1-yl]pyridin-3-yl}-2-(pyridin-4-yl)acetamide
N-{4-[4-chloro-5-cyclopropyl-3-(trifluoromethyl)-1H-pyrazol-1-yl]-3-fluorophenyl}-4-methylpyrimidine-5-carboxamide
1-{6-[3-cyclopropyl-5-(trifluoromethyl)-1H-pyrazol-1-yl]pyridin-3-yl}-3-(4-methylthiazol-5-yl)urea
6-[5-cyclopropyl-3-(trifluoromethyl)-1H-pyrazol-1-yl]-N-(2,3-difluorophenyl) nicotinamide
6-[5-cyclopropyl-3-(trifluoromethyl)-1H-pyrazol-1-yl]-N-(2,6-difluorophenyl) nicotinamide
N-{6-[4-chloro-5-cyclopropyl-3-(trifluoromethyl)-1H-pyrazol-1-yl]pyridin-3-yl}-4-methylthiazole-5-carboxamide
N-{2-[5-cyclopropyl-3-(trifluoromethyl)-1H-pyrazol-1-yl]pyrimidin-5-yl}-2,6-difluorobenzamide
N-{4-[5-(fluoromethyl)-3-(trifluoromethyl)-1H-pyrazol-1-yl]phenyl}-4-methylthiazole-5-carboxamide
N-{4-[5-(difluoromethyl)-3-(trifluoromethyl)-1H-pyrazol-1-yl]phenyl}-4-methylthiazole-5-carboxamide
3,5-dichloro-N-[4-(3,5-dicyclopropyl-1H-pyrazol-1-yl)-3-fluorophenyl]isonicotinamide
N-(2-chloro-6-fluorophenyl)-4-[5-cyclopropyl-3-(trifluoromethyl)-1H-pyrazol-1-yl]-3-fluorobenzamide
N-{2-[5-cyclopropyl-3-(trifluoromethyl)-1H-pyrazol-1-yl]pyrimidin-5-yl}-4-methylthiazole-5-carboxamide
N-{4-[5-cyclopropyl-3-(trifluoromethyl)-1H-pyrazol-1-yl]-3,5-difluorophenyl}-4-methylpyrimidine-5-carboxamide
{4-[5-cyclopropyl-3-(trifluoromethyl)-1H-pyrazol-1-yl]-3-fluorophenyl}-1-phenylcyclobutanecarboxamide
N-{4-[5-cyclopropyl-3-(trifluoromethyl)-1H-pyrazol-1-yl]-3-fluorophenyl}-4-methyloxazole-5-carboxamide
N-{2-[5-cyclopropyl-3-(trifluoromethyl)-1H-pyrazol-1-yl]pyrimidin-5-yl}-4-methylpyrimidine-5-carboxamide
4-[5-cyclopropyl-3-(trifluoromethyl)-1H-pyrazol-1-yl]-3-fluoro-N-(4-methylpyrimidin-5-yl) benzamide and
N-{4-[3-cyclopropyl-5-(difluoromethyl)-1H-pyrazol-1-yl]-3-fluorophenyl}-2,6-difluorobenzamide;
N-{4-[5-cyclopropyl-3-(difluoromethyl)-1H-pyrazol-1-yl]-3-fluorophenyl}-2,6-difluorobenzamide
N-[4-(3,5-dicyclopropyl-1H-pyrazol-1-yl)phenyl]-1H-benzo[d]imidazole-6-carboxamide
N-[4-(3,5-dicyclopropyl-1H-pyrazol-1-yl)phenyl]-1H-benzo[d][1,2,3]triazole-6-carboxamide
N-[4-(3,5-dicyclopropyl-1H-pyrazol-1-yl)phenyl]quinoline-6-carboxamide hydrochloride
N-[4-(3,5-dicyclopropyl-1H-pyrazol-1-yl)phenyl]quinoxaline-6-carboxamide
2-(1H-benzo[d]imidazol-1-yl)-N-[4-(3,5-dicyclopropyl-1H-pyrazol-1-yl)phenyl]acetamide
2-(1H-benzo[d][1,2,3]triazol-1-yl)-N-[4-(3,5-dicyclopropyl-1H-pyrazol-1-yl)phenyl]acetamide
N-[4-(3,5-dicyclopropyl-1H-pyrazol-1-yl)phenyl]-2-(1H-indol-3-yl)acetamide
N-[4-(3,5-dicyclopropyl-1H-pyrazol-1-yl)phenyl]-2-(imidazo[1,2-a]pyridin-2-yl)acetamide hydrochloride
N-[4-(3,5-dicyclopropyl-1H-pyrazol-1-yl)phenyl]-2-(quinolin-6-yl)acetamide:
N-[4-(3,5-dicyclopropyl-1H-pyrazol-1-yl)phenyl]-2-(quinolin-6-yl)acetamide hydrochloride
2-(1H-benzo[d][1,2,3]triazol-1-yl)-N-(4-(3,5-dicyclopropyl-1H-pyrazol-1-yl)-3-fluorophenyl)acetamide
N-[4-(3,5-dicyclopropyl-1H-pyrazol-1-yl)-3-fluorophenyl]-2-(quinolin-6-yl)acetamide hydrochloride
N-[6-(3,5-dicyclopropyl-1H-pyrazol-1-yl)pyridin-3-yl]quinoline-6-carboxamide dihydrochloride
N-[6-(3,5-dicyclopropyl-1H-pyrazol-1-yl)pyridin-3-yl]quinoxaline-6-carboxamide
2-(1H-benzo[d][1,2,3]triazol-1-yl)-N-[6-(3,5-dicyclopropyl-1H-pyrazol-1-yl)pyridin-3-yl]acetamide
N-[6-(3,5-dicyclopropyl-1H-pyrazol-1-yl)pyridin-3-yl]-2-(quinolin-6-yl)acetamidedihydrochloride
N-{4-[5-cyclopropyl-3-(trifluoromethyl)-1H-pyrazol-1-yl]phenyl}quinoline-6-carboxamide hydrochloride
N-{4-[5-cyclopropyl-3-(trifluoromethyl)-1H-pyrazol-1-yl]phenyl}quinoxaline-6-carboxamide
2-(1H-benzo[d]imidazol-1-yl)-N-{4-[5-cyclopropyl-3-(trifluoromethyl)-1H-pyrazol-1-yl]phenyl}acetamide
2-(1H-benzo[d][1,2,3]triazol-1-yl)-N-{4-[5-cyclopropyl-3-(trifluoromethyl)-1H-pyrazol-1-yl]phenyl}acetamide
2-(2H-benzo[d][1,2,3]triazol-2-yl)-N-{4-[5-cyclopropyl-3-(trifluoromethyl)-1H-pyrazol-1-yl]phenyl}acetamide
2-(3H-[1,2,3]triazolo[4,5-b]pyridin-3-yl)-N-{4-[5-cyclopropyl-3-(trifluoromethyl)-1H-pyrazol-1-yl]phenyl}acetamide
(S)-2-(3H-[1,2,3]triazolo[4,5-b]pyridin-3-yl)-N-{4-[5-cyclopropyl-3-(trifluoromethyl)-1H-pyrazol-1-yl]phenyl}propanamide
2-(6-amino-9H-purin-9-yl)-N-{4-[5-cyclopropyl-3-(trifluoromethyl)-1H-pyrazol-1-yl]phenyl}acetamide
N-(4-(5-cyclopropyl-3-(trifluoromethyl)-1H-pyrazol-1-yl)phenyl)-2-(1,3-dimethyl-2,6-dioxo-2,3-dihydro-1H-purin-7(6H)-yl) acetamide
N-{4-[5-cyclopropyl-3-(trifluoromethyl)-1H-pyrazol-1-yl)phenyl)-2-(imidazo[1,2-a]pyridin-2-yl)acetamide hydrochloride
N-{4-[5-cyclopropyl-3-(trifluoromethyl)-1H-pyrazol-1-yl]phenyl}-2-(quinolin-6-yl)acetamide hydrochloride
N-{4-[5-cyclopropyl-3-(trifluoromethyl)-1H-pyrazol-1-yl]phenyl}-2-(quinolin-6-yl)propanamide hydrochloride
N-{4-[5-cyclopropyl-3-(trifluoromethyl)-1H-pyrazol-1-yl]-3-fluorophenyl}-1H-benzo[d][1,2,3]triazole-6-carboxamide
2-(1H-benzo[d][1,2,3]triazol-1-yl)-N-{4-[5-cyclopropyl-3-(trifluoromethyl)-1H-pyrazol-1-yl]-3-fluorophenyl}acetamide
N-{6-[5-cyclopropyl-3-(trifluoromethyl)-1H-pyrazol-1-yl]pyridin-3-yl}-1H-benzo[d][1,2,3]triazole-5-carboxamide
2-(1H-benzo[d][1,2,3]triazol-1-yl)-N-{6-[5-cyclopropyl-3-(trifluoromethyl)-1H-pyrazol-1-yl]pyridin-3-yl}acetamide
2-(2H-benzo[d][1,2,3]triazol-2-yl)-N-{6-[5-cyclopropyl-3-(trifluoromethyl)-1H-pyrazol-1-yl]pyridin-3-yl}acetamide
N-{6-[5-cyclopropyl-3-(trifluoromethyl)-1H-pyrazol-1-yl]pyridin-3-yl}-2-(quinolin-6-yl)acetamide hydrochloride
2-(1H-benzo[d][1,2,3]triazol-1-yl)-N-{6-[4-chloro-5-cyclopropyl-3-(trifluoromethyl)-1H-pyrazol-1-yl]pyridin-3-yl}acetamide
4-[5-cyclopropyl-3-(trifluoromethyl)-1H-pyrazol-1-yl]-3-fluoro-N-(quinolin-6-ylmethyl) benzamide hydrochloride,
1-[4-(3,5-dicyclopropyl-1H-pyrazol-1-yl)phenyl]-3-(quinolin-6-yl)urea and pharmaceutically acceptable salts thereof.

In another preferred embodiment of any of the methods and/or compositions described herein, the CRAC inhibitor selected from
CM2489;
CM4620;
N-(5-(6-chloro-2,2-difluorobenzo[d][1,3]dioxol-5-yl)pyrazin-2-yl)-2-fluoro-6-methylbenzamide;
N-[4-[3,5-Bis(trifluoromethyl)-1H-pyrazol-1-yl]phenyl]-4-methyl-1,2,3-thiadiazole-5-carboxamide (YM-58483);
2,6-Difluoro-N-{5-[4-methyl-1-(5-methyl-thiazol-2-yl)-1,2,5,6-tetrahydro-pyridin-3-yl]-pyrazin-2-yl}-benzamid (R02959);
2,6-Difluoro-N-(1-(4-hydroxy-2-(trifluoromethyl)benzyl)-1H-pyrazol-3-yl)benzamide (GSK-7975A);
2,6-Difluoro-N-(1-(2-phenoxybenzyl)-1H-pyrazol-3-yl)benzamide (GSK5503A);
N-(2′,5′-Dimethoxy[1,1′-biphenyl]-4-yl)-3-fluoro-4-pyridinecarboxamide (Synta 66);
N-{6-[5-cyclopropyl-3-(trifluoromethyl)-1H-pyrazol-1-yl]pyridin-3-yl}-2-methylbenzamide;
N-{6-[5-cyclopropyl-3-(trifluoromethyl)-1H-pyrazol-1-yl]pyridin-3-yl}-3-fluoroisonicotinamide;
   and pharmaceutically acceptable salts thereof.

In another preferred embodiment of any of the methods and/or compositions described herein, the CRAC inhibitor is selected from
N-{6-[5-cyclopropyl-3-(trifluoromethyl)-1H-pyrazol-1-yl]pyridin-3-yl}-2-methyl benzamide (Compound A);
N-{6-[5-cyclopropyl-3-(trifluoromethyl)-1H-pyrazol-1-yl]pyridin-3-yl}-3-fluoroisonicotinamide;
   and pharmaceutically acceptable salts thereof.

In another preferred embodiment of any of the methods and/or compositions described herein, the corticosteroid is selected from dexamethasone, betamethasone, prednisolone, methyl prednisolone, prednisone, hydrocortisone, fluticasone, triamcinolone, budesonide or cortisone prednisolone, methylprednisolone, naflocort, deflazacort, halopredone acetate, budesonide, beclomethasone dipropionate, hydrocortisone, triamcinolone acetonide, fluocinolone acetonide, fluocinonide, clocortolone pivalate, methylprednisolone aceponate, dexamethasone palmitoate, tipredane, hydrocortisone aceponate, prednicarbate, alclometasone dipropionate, halometasone, methylprednisolone suleptanate, mometasone, mometasone furoate, mometasone furoate monohydrate nmexolone, prednisolone farnesylate, ciclesonide, deprodone propionate, fluticasone propionate, halobetasol propionate, loteprednol etabonate, betamethasone butyrate propionate, flunisolide, prednisone, dexamethasone sodium phosphate, triamcinolone, betamethasone 17-valerate, betamethasone, betamethasone dipropionate, hydrocortisone acetate, hydrocortisone sodium succinate, prednisolone sodium phosphate, hydrocortisone probutate, and pharmaceutically acceptable salts thereof.

In another preferred embodiment, the present invention provides a method of treating autoimmune, respiratory and inflammatory diseases and conditions comprising administering a combination comprising (i) a compound of formula (I), (IA) or (IB), or a pharmaceutically acceptable salt thereof and (ii) a corticosteroid selected from dexamethasone, betamethasone, prednisolone, methyl prednisolone, prednisone, mometasone furoate, mometasone furoate monohydrate, hydrocortisone, fluticasone, triamcinolone, budesonide or cortisone or a pharmaceutically acceptable salt thereof.

The present invention also relates to pharmaceutical composition comprising a Compound of formula (I), (IA) and (IB), a CRAC inhibitor and at least one corticosteroid, and to use of said pharmaceutical compositions for treating autoimmune, respiratory and inflammatory diseases and conditions.

The present invention also relates to pharmaceutical composition comprising a CRAC inhibitor selected from
CM2489;
CM4620;
N-(5-(6-chloro-2,2-difluorobenzo[d][1,3]dioxol-5-yl)pyrazin-2-yl)-2-fluoro-6-methylbenzamide;
N-[4-[3,5-Bis(trifluoromethyl)-1H-pyrazol-1-yl]phenyl]-4-methyl-1,2,3-thiadiazole-5-carboxamide (YM-58483);
2,6-Difluoro-N-{5-[4-methyl-1-(5-methyl-thiazol-2-yl)-1,2,5,6-tetrahydro-pyridin-3-yl]-pyrazin-2-yl}-benzamid (R02959);
2,6-Difluoro-N-(1-(4-hydroxy-2-(trifluoromethyl)benzyl)-1H-pyrazol-3-yl)benzamide (GSK-7975A);
2,6-Difluoro-N-(1-(2-phenoxybenzyl)-1H-pyrazol-3-yl)benzamide (GSK5503A);
N-(2′,5′-Dimethoxy[1,1′-biphenyl]-4-yl)-3-fluoro-4-pyridinecarboxamide (Synta 66);
N-{6-[5-cyclopropyl-3-(trifluoromethyl)-1H-pyrazol-1-yl]pyridin-3-yl}-2-methylbenzamide;
N-{6-[5-cyclopropyl-3-(trifluoromethyl)-1H-pyrazol-1-yl]pyridin-3-yl}-3-fluoroisonicotinamide;
   and pharmaceutically acceptable salts thereof; and
   at least one corticosteroid,
   and to the use of said pharmaceutical compositions for treating autoimmune, respiratory and inflammatory diseases and conditions.

In another preferred embodiment of any of the methods and/or compositions described herein, the compound of formula (I) is a CRAC inhibitor selected from
N-{6-[5-cyclopropyl-3-(trifluoromethyl)-1H-pyrazol-1-yl]pyridin-3-yl}-2-methyl benzamide (Compound A);
N-{6-[5-cyclopropyl-3-(trifluoromethyl)-1H-pyrazol-1-yl]pyridin-3-yl}-3-fluoroisonicotinamide;
   and pharmaceutically acceptable salts thereof;
   and the corticosteroid is selected from dexamethasone, betamethasone, prednisolone, methyl prednisolone, prednisone, mometasone, mometasone furoate, mometasone furoate monohydrate, hydrocortisone, fluticasone, triamcinolone, budesonide, cortisone or a pharmaceutically acceptable salt thereof.

In another preferred embodiment of any of the methods and/or compositions described herein, the CRAC modulator is N-{6-[5-cyclopropyl-3-(trifluoromethyl)-1H-pyrazol-1-yl]pyridin-3-yl}-2-methyl benzamide and the corticosteroid is dexamethasone.

In another preferred embodiment of any of the methods and/or compositions described herein the CRAC modulator is N-{6-[5-cyclopropyl-3-(trifluoromethyl)-1H-pyrazol-1-yl]pyridin-3-yl}-2-methyl benzamide and the corticosteroid is mometasone, mometasone furoate or mometasone furoate monohydrate.

In another preferred embodiment of any of the methods and/or compositions described herein the CRAC modulator is N-{6-[5-cyclopropyl-3-(trifluoromethyl)-1H-pyrazol-1-yl]pyridin-3-yl}-2-methyl benzamide and the corticosteroid is fluticasone.

In another aspect, the present invention relates to a kit for treating an autoimmune, respiratory or inflammatory disease or condition, the kit comprising:

(i) a CRAC modulator or a pharmaceutically acceptable salt thereof, and (ii) a corticosteroid or a pharmaceutically acceptable salt thereof, either in a single pharmaceutical composition or in separate pharmaceutical compositions according to any of the embodiments described herein,

(ii) optionally, instructions for treating the autoimmune, respiratory or inflammatory disease or condition with the CRAC modulator and corticosteroid; and

(iii) optionally, a container for placing the pharmaceutical composition or pharmaceutical compositions

### BRIEF DESCRIPTION OF THE DRAWINGS

FIG. 1A is a scattered graph depicting the effect of Compound A on the IC₅₀ of dexamethasone (Dex) on IL-8 concentrations in H₂O₂ treated U937 cells.

FIG. 1B is a bar graph depicting the effect of Compound A on the IC₅₀ of Dexamethasone (Dex) on IL-8 concentrations in H₂O₂ treated U937 cells.

FIG. 2 depicts the effect of Compound A on IL-1β, IL-6, and GM-CSF release in cells isolated from asthma patients and healthy subjects.

FIG. 3 depicts the effect of Compound A in combination with fluticasone (F) on IL-1β, IL-6, and GM-CSF release in cells isolated from asthma patients and healthy subjects.

### DETAILED DESCRIPTION OF THE INVENTION

Unless defined otherwise, all technical and scientific terms used herein have the same meaning as is commonly understood in the field to which the subject matter belongs. In the event that there is a plurality of definitions for terms herein, those in this section prevail.

Abbreviations used herein have their conventional meaning within the chemical and biological arts, unless otherwise indicated.

It is to be understood that the foregoing general description and the following detailed description are exemplary and explanatory only and are not restrictive of any subject matter. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Definition of standard chemistry and molecular biology terms may be found in reference works, including, but not limited to, Carey and Sundberg “ADVANCED ORGANIC CHEMISTRY 4^{th} edition” Vols. A (2000) and B (2001), Plenum Press, New York and “MOLECULAR BIOLOGY OF THE CELL 5^{th} edition” (2007), Garland Science, New York. Unless otherwise indicated, conventional methods of mass spectroscopy, NMR, HPLC, protein chemistry, biochemistry, recombinant DNA techniques and pharmacology are contemplated within the scope of the embodiments disclosed herein.

Unless specific definitions are provided, the nomenclature employed in connection with, and the laboratory procedures and techniques of, analytical chemistry, and medicinal and pharmaceutical chemistry described herein are those generally used. In some embodiments, standard techniques are used for chemical analyses, pharmaceutical preparation, formulation, and delivery, and treatment of patients. In other embodiments, standard techniques are used for recombinant DNA, oligonucleotide synthesis, and tissue culture and transformation (e.g., electroporation, lipofection). In certain embodiments, reactions and purification techniques are performed e.g., using kits of manufacturer's specifications or as described herein. The foregoing techniques and procedures are generally performed of conventional methods and as described in various general and more specific references that are cited and discussed throughout the present specification.

Additionally, the present invention also includes compounds which differ only in the presence of one or more isotopically enriched atoms, for example replacement of hydrogen with deuterium, and the like.

The term “subject” or “patient” encompasses mammals and non-mammals Examples of mammals include, but are not limited to, any member of the Mammalian class: humans, non-human primates such as chimpanzees, and other apes and monkey species; farm animals such as cattle, horses, sheep, goats, and swine; domestic animals such as rabbits, dogs, and cats; and laboratory animals including rodents, such as rats, mice and guinea pigs. Examples of non-mammals include, but are not limited to, birds, fish and the like. In one embodiment of the methods and compositions provided herein, the mammal is a human

The terms “treat,” “treating” and “treatment,” as used herein, include alleviating, abating or ameliorating a disease, disorder or condition symptoms, preventing additional symptoms, ameliorating or preventing the underlying causes of symptoms, inhibiting the disease, disorder or condition, e.g., arresting the development of the disease, disorder or condition, relieving the disease, disorder or condition, causing regression of the disease, disorder or condition, relieving a condition caused by the disease, disorder or condition, or stopping the symptoms of the disease, disorder or condition either prophylactically and/or therapeutically.

As used herein, the term “target protein” refers to a protein or a portion of a protein capable of being bound by, or interacting with, a compound described herein, such as a compound capable of modulating a STIM protein and/or an Orai protein. In certain embodiments, a target protein is a STIM protein. In other embodiments, a target protein is an Orai protein. In yet other embodiments, the compound described herein targets both STIM and Orai proteins.

The term “STIM protein” refers to any protein situated in the endoplasmic reticular or plasma membrane which activates an increase in rate of calcium flow into a cell by a CRAC channel (STIM refers to a stromal interaction molecule). As used herein, “STIM protein” includes, but is not limited to, mammalian STIM-1, such as human and rodent (e.g., mouse) STIM-1, Drosophila melanogaster D-STIM, C. elegans C-STIM, Anopheles gambiae STIM and mammalian STIM-2, such as human and rodent (e.g., mouse) STIM-2. As described herein, such proteins have been identified as being involved in, participating in and/or providing for store-operated calcium entry or modulation thereof, cytoplasmic calcium buffering and/or modulation of calcium levels in or movement of calcium into, within or out of intracellular calcium stores (e.g., endoplasmic reticulum).

It will be appreciated by “activate” or “activation” it is meant the capacity of a STIM protein to up-regulate, stimulate, enhance or otherwise facilitate calcium flow into a cell by a CRAC channel. It is envisaged that cross-talk between the STIM protein and the CRAC channel may occur by either a direct or indirect molecular interaction. Suitably, the STIM protein is a transmembrane protein which is associated with, or in close proximity to, a CRAC channel.

As used herein, an “Orai protein” includes Orai1 (SEQ ID NO: 1 as described in WO 07/081804), Orai2 (SEQ ID NO: 2 as described in WO 07/081804), or Orai3 (SEQ ID NO: 3 as described in WO 07/081804). Orai1 nucleic acid sequence corresponds to GenBank accession number NM-032790, Orai2 nucleic acid sequence corresponds to GenBank accession number BC069270 and Orai3 nucleic acid sequence corresponds to GenBank accession number NM-152288. As used herein, Orai refers to any one of the Orai genes, e.g., Orai1, Orai2, and Orai3 (see Table I of WO 07/081804). As described herein, such proteins have been identified as being involved in, participating in and/or providing for store-operated calcium entry or modulation thereof, cytoplasmic calcium buffering and/or modulation of calcium levels in or movement of calcium into, within or out of intracellular calcium stores (e.g., endoplasmic reticulum). In alternative embodiments, an Orai protein may be labelled with a tag molecule, by way of example only, an enzyme fragment, a protein (e.g. c-myc or other tag protein or fragment thereof), an enzyme tag, a fluorescent tag, a fluorophore tag, a chromophore tag, a Raman-activated tag, a chemiluminescent tag, a quantum dot marker, an antibody, a radioactive tag, or combination thereof.

The term “fragment” or “derivative” when referring to a protein (e.g. STIM, Orai) means proteins or polypeptides which retain essentially the same biological function or activity in at least one assay as the native protein(s). For example, the fragment or derivative of the referenced protein preferably maintains at least about 50% of the activity of the native protein, at least 75% of the activity of the native protein or at least about 95% of the activity of the native protein, as determined, e.g., by a calcium influx assay.

As used herein, “amelioration” refers to an improvement in a disease or condition or at least a partial relief of symptoms associated with a disease or condition. As used herein, amelioration of the symptoms of a particular disease, disorder or condition by administration of a particular compound or pharmaceutical composition refers to any lessening of severity, delay in onset, slowing of progression, or shortening of duration, whether permanent or temporary, lasting or transient that are attributed to or associated with administration of the compound or composition.

The term “modulate,” as used herein, means to interact with a target protein either directly or indirectly so as to alter the activity of the target protein, including, by way of example only, to inhibit the activity of the target, or to limit or reduce the activity of the target.

As used herein, the term “modulator” refers to a compound that alters an activity of a target (e.g., a target protein). For example, in some embodiments, a modulator causes an increase or decrease in the magnitude of a certain activity of a target compared to the magnitude of the activity in the absence of the modulator. In certain embodiments, a modulator is an inhibitor, which decreases the magnitude of one or more activities of a target. In certain embodiments, an inhibitor completely prevents one or more activities of a target.

As used herein, “modulation” with reference to intracellular calcium refers to any alteration or adjustment in intracellular calcium including but not limited to alteration of calcium concentration in the cytoplasm and/or intracellular calcium storage organelles, e.g., endoplasmic reticulum, or alteration of the kinetics of calcium fluxes into, out of and within cells. In aspect, modulation refers to reduction.

The terms “inhibits,” “inhibiting” or “inhibitor” of SOC channel activity or CRAC channel activity, as used herein, refer to inhibition of store operated calcium channel activity or calcium release activated calcium channel activity.

The term “acceptable” with respect to a formulation, composition or ingredient, as used herein, means having no persistent detrimental effect on the general health of the subject being treated.

The term “pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

Pharmaceutically acceptable salts forming part of this invention include salts derived from inorganic bases such as Li, Na, K, Ca, Mg, Fe, Cu, Zn, and Mn; salts of organic bases such as N,N′-diacetylethylenediamine, glucamine, triethylamine, choline, hydroxide, dicyclohexylamine, metformin, benzylamine, trialkylamine, thiamine, and the like; chiral bases like alkylphenylamine, glycinol, and phenyl glycinol, salts of natural amino acids such as glycine, alanine, valine, leucine, isoleucine, norleucine, tyrosine, cystine, cysteine, methionine, proline, hydroxy proline, histidine, omithine, lysine, arginine, and serine; quaternary ammonium salts of the compounds of invention with alkyl halides, and alkyl sulphates such as Mel and (Me)₂SO₄, non-natural amino acids such as D-isomers or substituted amino acids; guanidine, substituted guanidine wherein the substituents are selected from nitro, amino, alkyl, alkenyl, alkynyl, ammonium or substituted ammonium salts and aluminum salts. Salts may include acid addition salts where appropriate which are, sulphates, nitrates, phosphates, perchlorates, borates, hydrohalides, acetates, tartrates, maleates, citrates, fumarates, succinates, palmoates, methanesulphonates, benzoates, salicylates, benzenesulfonates, ascorbates, glycerophosphates, and ketoglutarates. Pharmaceutically acceptable solvates may be hydrates or comprise other solvents of crystallization such as alcohols.

The term “pharmaceutical composition” refers to a mixture of a compound of the present invention with other chemical components, such as, but not limited to, one or more carriers, stabilizers, diluents, dispersing agents, suspending agents, thickening agents, and/or excipients.

The compounds and pharmaceutical compositions of the present invention can be administered by various routes of administration including, but not limited to, intravenous, oral, aerosol, parenteral, ophthalmic, pulmonary and topical administration.

The terms “effective amount” or “therapeutically effective amount,” as used herein, refer to a sufficient amount of an agent or a compound being administered which will relieve to some extent one or more of the symptoms of the disease or condition being treated. The result is reduction and/or alleviation of the signs, symptoms, or causes of a disease, or any other desired alteration of a biological system. For example, an “effective amount” for therapeutic uses is the amount of a compound of the present invention required to provide a clinically significant decrease in disease symptoms. In some embodiments, an appropriate “effective” amount in any individual case is determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “carrier,” as used herein, refers to relatively nontoxic chemical compounds or agents that facilitate the incorporation of a compound into cells or tissues.

The term “diluent” refers to chemical compounds that are used to dilute the compound of interest prior to delivery. In some embodiments, diluents are used to stabilize compounds because they provide a more stable environment. Salts dissolved in buffered solutions (which also provide pH control or maintenance) are utilized as diluents, including, but not limited to a phosphate buffered saline solution.

As used herein, “intracellular calcium” refers to calcium located in a cell without specification of a particular cellular location. In contrast, “cytosolic” or “cytoplasmic” with reference to calcium refers to calcium located in the cell cytoplasm.

As used herein, an effect on intracellular calcium is any alteration of any aspect of intracellular calcium, including, but not limited to, an alteration in intracellular calcium levels and location and movement of calcium into, out of or within a cell or intracellular calcium store or organelle. For example, in some embodiments, an effect on intracellular calcium is an alteration of the properties, such as, for example, the kinetics, sensitivities, rate, amplitude, and electrophysiological characteristics, of calcium flux or movement that occurs in a cell or portion thereof. In some embodiments, an effect on intracellular calcium is an alteration in any intracellular calcium-modulating process, including, store-operated calcium entry, cytosolic calcium buffering, and calcium levels in or movement of calcium into, out of or within an intracellular calcium store. Any of these aspects are assessed in a variety of ways including, but not limited to, evaluation of calcium or other ion (particularly cation) levels, movement of calcium or other ion (particularly cation), fluctuations in calcium or other ion (particularly cation) levels, kinetics of calcium or other ion (particularly cation) fluxes and/or transport of calcium or other ion (particularly cation) through a membrane. An alteration is any such change that is statistically significant. Thus, for example, in some embodiments, if intracellular calcium in a test cell and a control cell is said to differ, such differences are a statistically significant difference.

Modulation of intracellular calcium is any alteration or adjustment in intracellular calcium including but not limited to alteration of calcium concentration or level in the cytoplasm and/or intracellular calcium storage organelles, e.g., endoplasmic reticulum, alteration in the movement of calcium into, out of and within a cell or intracellular calcium store or organelle, alteration in the location of calcium within a cell, and alteration of the kinetics, or other properties, of calcium fluxes into, out of and within cells. In some embodiments, intracellular calcium modulation involves alteration or adjustment, e.g. reduction or inhibition, of store-operated calcium entry, cytosolic calcium buffering, calcium levels in or movement of calcium into, out of or within an intracellular calcium store or organelle, and/or basal or resting cytosolic calcium levels. The modulation of intracellular calcium involves an alteration or adjustment in receptor-mediated ion (e.g., calcium) movement, second messenger-operated ion (e.g., calcium) movement, calcium influx into or efflux out of a cell, and/or ion (e.g., calcium) uptake into or release from intracellular compartments, including, for example, endosomes and lysosomes.

As used herein, “involved in,” with respect to the relationship between a protein and an aspect of intracellular calcium or intracellular calcium regulation means that when expression or activity of the protein in a cell is reduced, altered or eliminated, there is a concomitant or associated reduction, alteration or elimination of one or more aspects of intracellular calcium or intracellular calcium regulation. Such an alteration or reduction in expression or activity occurs by virtue of an alteration of expression of a gene encoding the protein or by altering the levels of the protein. A protein involved in an aspect of intracellular calcium, such as, for example, store-operated calcium entry, thus, are one that provides for or participates in an aspect of intracellular calcium or intracellular calcium regulation. For example, a protein that provides for store-operated calcium entry are a STIM protein and/or an Orai protein.

As used herein, “cation entry” or “calcium entry” into a cell refers to entry of cations, such as calcium, into an intracellular location, such as the cytoplasm of a cell or into the lumen of an intracellular organelle or storage site. Thus, in some embodiments, cation entry is, for example, the movement of cations into the cell cytoplasm from the extracellular medium or from an intracellular organelle or storage site, or the movement of cations into an intracellular organelle or storage site from the cytoplasm or extracellular medium. Movement of calcium into the cytoplasm from an intracellular organelle or storage site is also referred to as “calcium release” from the organelle or storage site.

As used herein, “immune cells” include cells of the immune system and cells that perform a function or activity in an immune response, such as, but not limited to, T-cells, B-cells, lymphocytes, macrophages, dendritic cells, neutrophils, eosinophils, basophils, mast cells, plasma cells, white blood cells, antigen presenting cells and natural killer cells.

“Store operated calcium entry” or “SOCE” refers to the mechanism by which release of calcium ions from intracellular stores is coordinated with ion influx across the plasma membrane.

A “therapeutic effect,” as that term is used herein encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

“Signal transduction” is a process during which stimulatory or inhibitory signals are transmitted into and within a cell to elicit an intracellular response. A modulator of a signal transduction pathway refers to a compound which modulates the activity of one or more cellular proteins mapped to the same specific signal transduction pathway. A modulator may augment (agonist) or suppress (antagonist) the activity of a signaling molecule.

“Inflammatory response” as used herein is characterized by redness, heat, swelling and pain (i.e., inflammation) and typically involves tissue injury or destruction. An inflammatory response is usually a localized, protective response elicited by injury or destruction of tissues, which serves to destroy, dilute or wall off (sequester) both the injurious agent and the injured tissue. Inflammatory responses are notably associated with the influx of leukocytes and/or leukocyte (e.g., neutrophil) chemotaxis. Inflammatory responses may result from infection with pathogenic organisms and viruses, noninfectious means such as trauma or reperfusion following myocardial infarction or stroke, immune responses to foreign antigens, and autoimmune diseases. Inflammatory responses amenable to treatment with the methods and compounds according to the invention encompass conditions associated with reactions of the specific defense system as well as conditions associated with reactions of the non-specific defense system.

The therapeutic methods of the invention include methods for the treatment of conditions associated with inflammatory cell activation. “Inflammatory cell activation” refers to the induction by a stimulus (including, but not limited to, cytokines, antigens or auto-antibodies) of a proliferative cellular response, the production of soluble mediators (including but not limited to cytokines, oxygen radicals, enzymes, prostanoids, or vasoactive amines), or cell surface expression of new or increased numbers of mediators (including, but not limited to, major histocompatibility antigens or cell adhesion molecules) in inflammatory cells (including, but not limited to, monocytes, macrophages, T lymphocytes, B lymphocytes, granulocytes (polymorphonuclear leukocytes including neutrophils, basophils, and eosinophils) mast cells, dendritic cells, Langerhans cells, and endothelial cells). It will be appreciated by persons skilled in the art that the activation of one or a combination of these phenotypes in these cells can contribute to the initiation, perpetuation, or exacerbation of an inflammatory condition.

“Autoimmune disease” as used herein refers to any group of disorders in which tissue injury is associated with humoral or cell-mediated responses to the body's own constituents.

“Transplant rejection” as used herein refers to an immune response directed against grafted tissue (including organs or cells (e.g., bone marrow), characterized by a loss of function of the grafted and surrounding tissues, pain, swelling, leukocytosis, and thrombocytopenia.

“Allergic disease” as used herein refers to any symptoms, tissue damage, or loss of tissue function resulting from allergy.

“Arthritic disease” as used herein refers to any disease that is characterized by inflammatory lesions of the joints attributable to a variety of etiologies.

“Dermatitis” as used herein refers to any of a large family of diseases of the skin that are characterized by inflammation of the skin attributable to a variety of etiologies.

The compounds of the present invention are also useful in combination (administered together or sequentially) with one or more steroidal anti-inflammatory drugs, non-steroidal anti-inflammatory drugs (NSAIDs), immune selective anti-inflammatory Derivatives (ImSAIDs), or any combination thereof.

The term “co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present.

According to the present invention, the compound of formula (I), (IA) and (IB) or a hydrate, a pharmaceutically acceptable salt or a solvate thereof, can also be administered in combination with one or more other active principles useful in one of the pathologies mentioned above, for example an anti-emetic, analgesic, anti-inflammatory or anti-cachexia agent.

It is also possible to combine the compositions of the present invention with a radiation treatment.

It is also possible to combine the compositions of the present invention with surgery, including either pre, post, or during period of surgery.

These treatments can be administered simultaneously, separately, sequentially and/or spaced in time.

The method of combing a CRAC inhibitor with a corticosteroid, as described in any of the embodiments herein, show an activity which is significantly higher than (a synergistic activity) the activity that would have been expected knowing the individual activities of each of the CRAC inhibitor or the corticosteroid alone.

The method of combing the CRAC inhibitor with a corticosteroid, as described in any of the embodiments herein, show an activity even when corticosteroid alone is insensitive as a single agent.

Thus, the method of present invention should allow for treating autoimmune, respiratory and inflammatory diseases and conditions with a smaller amount of active compounds and/or should allow for treating autoimmune, respiratory and inflammatory diseases and conditions for a longer period of time as well in a more efficient way.

The pharmaceutical compositions according to the present invention show an activity which is significantly higher than the activity that would have been expected knowing the individual activities of each of the components. Thus, the pharmaceutical compositions should allow for treating respiratory and inflammatory diseases and conditions with a smaller amount of active compounds and/or should allow for treating respiratory and inflammatory diseases and conditions in a more efficient way.

Therefore, the present invention further relates to a pharmaceutical composition according to the invention for use in the treatment of autoimmune, respiratory and inflammatory diseases and conditions.

Another embodiment of the present invention relates to a method of treating autoimmune respiratory and inflammatory diseases and conditions, comprising administering a therapeutically effective amount of a pharmaceutical composition according to the present invention to a patient in need thereof.

Another embodiment of the present invention relates to the use of a pharmaceutical composition according to the invention for making a medicament for treating autoimmune, respiratory and inflammatory diseases and conditions.

In the pharmaceutical compositions according to the present invention the CRAC inhibitor may be contained in a form selected from solvates, hydrates or salts with pharmacologically acceptable acids or bases.

In the pharmaceutical compositions according to the present invention the Corticosteroid may be contained in a form selected from solvates, hydrates or salts with pharmacologically acceptable acids or bases.

Another embodiment of the present invention is a method of treating an immune system-related disease (e.g., an autoimmune disease), a disease or disorder involving inflammation (e.g., asthma, chronic obstructive pulmonary disease, rheumatoid arthritis, inflammatory bowel disease, glomerulonephritis, neuro-inflammatory diseases, multiple sclerosis, uveitis and disorders of the immune system), cancer or other proliferative disease, a hepatic disease or disorder, or a renal disease or disorder. The method includes administering an effective amount of one or more compositions according to any of the embodiments described herein.

Examples of immune disorders which can be treated by the compositions of the present invention include, but are not limited to, psoriasis, rheumatoid arthritis, vasculitis, inflammatory bowel disease, dermatitis, osteoarthritis, asthma, inflammatory muscle disease, allergic rhinitis, vaginitis, interstitial cystitis, scleroderma, osteoporosis, eczema, allogeneic or xenogeneic transplantation (organ, bone marrow, stem cells and other cells and tissues) graft rejection, graft-versus-host disease, lupus erythematosus, inflammatory disease, type I diabetes, pulmonary fibrosis, dermatomyositis, Sjogren's syndrome, thyroiditis (e.g., Hashimoto's and autoimmune thyroiditis), myasthenia gravis, autoimmune hemolytic anemia, multiple sclerosis, cystic fibrosis, Idiopathic pulmonary fibrosis (IPF), chronic relapsing hepatitis, primary biliary cirrhosis, allergic conjunctivitis and atopic dermatitis.

When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary from, for example, between 1% and 15% of the stated number or numerical range. The term “comprising” (and related terms such as “comprise” or “comprises” or “having” or “including”) includes those embodiments, for example, an embodiment of any composition of matter, composition, method, or process, or the like, that “consist of” or “consist essentially of” the described features.

### Pharmaceutical, Compositions

The invention provides a pharmaceutical composition comprising a CRAC inhibitor and at least one corticosteroid (according to any of the embodiments described herein) and, optionally, one or more pharmaceutically acceptable carriers or excipients.

In one embodiment, the pharmaceutical composition includes a therapeutically effective amount of CRAC inhibitor and at least one corticosteroid (according to any of the embodiments described herein). The pharmaceutical composition may include one or more additional active ingredients as described herein.

The pharmaceutical carriers and/or excipients may be selected from, for example, diluents, fillers, salts, disintegrants, binders, lubricants, glidants, wetting agents, controlled release matrices, colorants, flavorings, buffers, stabilizers, solubilizers, and any combination thereof.

The pharmaceutical compositions of the present invention can be administered alone or in combination with one or more other active agents. Where desired, the subject compounds and other agent(s) may be mixed into a preparation or both components may be formulated into separate preparations to use them in combination separately or at the same time.

The pharmaceutical compositions of the present invention can be administered together or in a sequential manner with one or more other active agents. Where desired, the subject compounds and other agent(s) may be co-administered or both components may be administered in a sequence to use them as a combination.

The compounds and pharmaceutical compositions of the present invention can be administered by any route that enables delivery of the compounds to the site of action, such as orally, intranasally, topically (e.g., transdermally), intraduodenally, parenterally (including intravenously, intraarterially, intramuscularally, intravascularally, intraperitoneally or by injection or infusion), intradermally, by intramammary, intrathecally, intraocularly, retrobulbarly, intrapulmonary (e.g., aerosolized drugs) or subcutaneously (including depot administration for long term release e.g., embedded-under the-splenic capsule, brain, or in the cornea), sublingually, anally, rectally, vaginally, or by surgical implantation (e.g., embedded under the splenic capsule, brain, or in the cornea).

The compositions can be administered in solid, semi-solid, liquid or gaseous form, or may be in dried powder, such as lyophilized form. The pharmaceutical compositions can be packaged in forms convenient for delivery, including, for example, solid dosage forms such as capsules, sachets, cachets, gelatine, papers, tablets, suppositories, pellets, pills, troches, and lozenges. The type of packaging will generally depend on the desired route of administration. Implantable sustained release formulations are also contemplated, as are transdermal formulations.

The amount of the compound to be administered is dependent on the mammal being treated, the severity of the disorder or condition, the rate of administration, the disposition of the compound and the discretion of the prescribing physician. However, an effective dosage of the CRAC modulator and/or the corticosteroid is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In one embodiment, an effective dosage of the CRAC modulator is in the range of about 0.001 to about 100 mg per kg body weight per day, preferably about 1 to about 35 mg/kg/day, in single or divided doses. In another embodiment, an effective dosage of the corticosteroid is in the range of about 0.001 to about 100 mg per kg body weight per day, preferably about 1 to about 35 mg/kg/day, in single or divided doses. An effective amount of the CRC modulator and/or corticosteroid, or a composition containing both may be administered in either single or multiple doses (e.g., twice or three times a day).

In one embodiment, the pharmaceutical compositions described herein comprise a CRAC modulator and a corticosteroid in a ratio of between about 100:1 and about 1:100 by weight, such as between about 50:1 and about 1:50 by weight or between about 1:10 and about 10:1 by weight, or between about 1:5 and about 5:1 by weight.

In one embodiment, the pharmaceutical compositions described herein comprise from about 0.01 mg to about 1000 mg, such as from about 0.01 mg to about 500 mg, from about 0.01 mg to about 250 mg or from about 0.01 mg to about 100 mg of a CRAC modulator and from about 0.01 mg to about 1000 mg, such as from about 0.01 mg to about 500 mg, from about 0.01 mg to about 250 mg or from about 0.01 mg to about 100 mg of at least one corticosteroid.

In another embodiment, any of the pharmaceutical compositions described herein comprise from about 0.01 mg to about 1000 mg, such as from about 10 mg to about 500 mg, from about 50 mg to about 250 mg or from about 50 mg to about 100 mg of a CRAC modulator.

In another embodiment, any of the pharmaceutical compositions described herein comprise from about 10 mg to about 500 mg of a CRAC modulator.

In another embodiment, any of the pharmaceutical compositions described herein comprise from about 0.01 mg to about 100 mg of a corticosteroid.

In one embodiment of any of the pharmaceutical compositions described herein, the corticosteroid is selected from dexamethasone, betamethasone, prednisolone, methyl prednisolone, prednisone, hydrocortisone, fluticasone, triamcinolone, triamcinolone acetonide, budesonide, cortisone prednisolone, methylprednisolone, naflocort, deflazacort, halopredone acetate, budesonide, beclomethasone dipropionate, hydrocortisone, triamcinolone acetonide, fluocinolone acetonide, fluocinonide, clocortolone pivalate, clocortolone acetate, clocortolone caproate, methylprednisolone aceponate, dexamethasone palmitoate, tipredane, hydrocortisone, hydrocortisone butyrate, hydrocortisone aceponate, prednicarbate, alclometasone dipropionate, halometasone, methylprednisolone suleptanate, methylprednisolone sodium succinate, methylprednisolone acetate, mometasone, mometasone furoate, mometasone furoate monohydrate, rimexolone, prednisolone farnesylate, ciclesonide, deprodone propionate, fluticasone propionate, halobetasol propionate, loteprednol etabonate, betamethasone butyrate propionate, betamethasone sodium phosphate, betamethasone acetate, flunisolide, Flunisolide Hemihydrate, prednisone, dexamethasone sodium phosphate, betamethasone 17-valerate, betamethasone, betamethasone dipropionate, hydrocortisone acetate, hydrocortisone sodium succinate, prednisolone sodium phosphate, hydrocortisone probutate, and any combination thereof.

More preferably, the corticosteroid is selected from dexamethasone, betamethasone, prednisolone, methyl prednisolone, prednisone, hydrocortisone, fluticasone, triamcinolone, budesonide, cortisone, mometasone, mometasone furoate, mometasone furoate monohydrate, and any combination thereof.

One particular embodiment of the present invention relates to a pharmaceutical composition according to any embodiment of the present invention, wherein the corticosteroid is fluticasone.

Another particular embodiment of the present invention relates to pharmaceutical composition according to the present invention, wherein the corticosteroid is budesonide

Yet another particular embodiment of the present invention relates to a pharmaceutical composition according to any embodiment of the present invention, wherein the corticosteroid is prednisolone.

Yet another particular embodiment of the present invention relates to a pharmaceutical composition according to any embodiment of the present invention, wherein the corticosteroid is mometasone, mometasone furoate or mometasone furoate monohydrate.

Yet another particular embodiment of the present invention relates to a pharmaceutical composition according to any embodiment of the present invention, wherein the corticosteroid is dexamethasone.

A further embodiment of the present invention relates to a method of treating an indication selected from respiratory diseases and conditions such as diseases of the airways and lungs which are accompanied by increased or altered production of mucus and/or inflammatory and/or obstructive diseases of the airways such as acute bronchitis, chronic bronchitis, chronic obstructive bronchitis (COPD), cough, pulmonary emphysema, allergic or non-allergic rhinitis or sinusitis, chronic sinusitis or rhinitis, nasal polyposis, chronic rhinosinusitis, acute rhinosinusitis, asthma, allergic bronchitis, alveolitis, Farmer's disease, hyperreactive airways, bronchitis or pneumonitis caused by infection, e.g. by bacteria or viruses or helminthes or fungi or protozoons or other pathogens, pediatric asthma, bronchiectasis, pulmonary fibrosis, adult respiratory distress syndrome, bronchial and pulmonary edema, bronchitis or pneumonitis or interstitial pneumonitis caused by different origins, e.g. aspiration, inhalation of toxic gases, vapors, bronchitis or pneumonitis or interstitial pneumonitis caused by heart failure, X-rays, radiation, chemotherapy, bronchitis or pneumonitis or interstitial pneumonitis associated with collagenosis, e.g. lupus erythematodes, systemic scleroderma, lung fibrosis, idiopathic pulmonary lung fibrosis (IPF), interstitial lung diseases or interstitial pneumonitis of different origin, including asbestosis, silicosis, M. Boeck or sarcoidosis, granulomatosis, cystic fibrosis or mucoviscidosis, or a-1-antitrypsin deficiency; or selected from inflammatory diseases and conditions such as inflammatory diseases of the gastrointestinal tract of various origins such as inflammatory pseudopolyps, Crohn's disease, ulcerative colitis, inflammatory diseases of the joints, such as rheumatoid arthritis, or allergic inflammatory diseases of the oro-nasopharynx, skin or the eyes, such as atopic dermatitis, seasonal and perenial, chronic uritcaria, hives of unknown cause and allergic conjunctivitis; and in particular selected from asthma, allergic and non-allergic rhinitis, COPD and atopic dermatitis; the method comprising administering a therapeutically effective amount of a pharmaceutical composition according to any of the embodiments of the present invention to a patient in need thereof.

A further embodiment of the present invention relates to the use of a pharmaceutical composition according to any of the embodiments of the present invention for making a medicament for treating respiratory and/or inflammatory diseases and conditions, particularly wherein the respiratory and/or inflammatory diseases or conditions are selected from asthma, allergic and non-allergic rhinitis, COPD and atopic dermatitis.

A further embodiment of the present invention relates to a pharmaceutical composition according to any of the embodiments of the present invention for use in the treatment of respiratory and inflammatory diseases and conditions, particularly wherein the respiratory and inflammatory diseases or conditions are selected from asthma, allergic and non-allergic rhinitis, COPD and atopic dermatitis.

The present invention is now further illustrated by means of the following non-limiting biological examples.

### BIOLOGICAL EXAMPLES

As described in the following examples, Compound A is Example 104 of International Publication No. WO 2011/042797, which is hereby incorporated by reference.

### Example 1: H₂O₂ Induced Corticosteroid Insensitivity in U937 Cells

### Test Procedure

U937 cells were maintained in RPMI-1640 with 15 mM glutamine. 6×10⁶ cells were taken in a T-25 flask with 12 ml of fresh medium and treated with 1 μM of Compound A and incubated at 37° C. and 5% CO₂ for 30 min.

H₂O₂ was added at a final concentration of 200 μM to the above cells and incubated for 2 h.

Cells were pelleted and resuspended in serum free media and seeded on to a 96-well plate at 0.15×10⁶ cells per well in 100 μl.

50 μl of 3× dexamethasone at desired concentrations was added and incubated for 45 min

50 μl of 4× concentration of TNF-α was added such that the final concentration was 10 ng/ml, to induce IL-8 and incubated for 18 h.

Supernatant was collected and IL-8 was estimated by ELISA.

### Cytokine Assay

IL-8 strips were plated with fresh or thawed supernatants and incubated at room temperature for 2 h or overnight at 4° C.

Contents were discarded and strips were washed with 200 μl of wash buffer per well for 15 s for a total of 5 times.

Strips were blotted dry and 100 μl per well of 1× detection antibody was added and incubated at room temperature for 1 h.

Contents were discarded and strips were washed with 200 μl of wash buffer per well for 15 s for a total of 5 times.

Strips were blotted dry and 100 μl per well of 1× Avidin-HRP antibody was added and incubated at room temperature for 30 min.

Contents were discarded and the strips were washed with 200 μl per well of wash buffer for 15 s for a total of 5 times.

100 μl per well of TMB substrate were added and incubated at room temperature for 5-15 min.

The reaction was stopped by adding 50 μl per well of 2N H₂SO₄.

Absorbance was read on a plate reader at A₄₅₀ nm and A₅₇₀ nm.

### Results

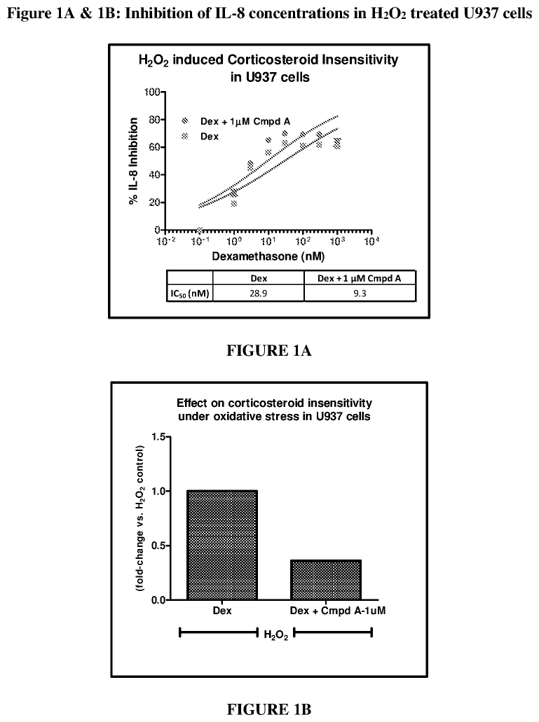

As depicted in FIG. 1A, Compound A (Cmpd A) decreased the IC50 of dexamethasone (Dex) on IL-8 concentrations in H₂O₂ treated U937 cells indicating significant potentiation of dexamethasone activity.

Addition of 1 μM of Compound A reversed H₂O₂-induced dexamethasone insensitivity in U937 macrophages manifested by 3-fold reduction in IC₅₀ for IL-8 release (FIG. 1B).

### Example 2: General Description Related to Patient Identification, Isolation of Mononuclear Cells from Healthy and Asthmatic Patients for In-Vitro Testing of Compound A as a Single Agent or in Combination with a Corticosteroid

Patients were classified into two groups: A) healthy subjects—patients with normal lung function and who did not smoke; and B) asthmatics—patients under iCS/LABA treatment diagnosed according to GINA (Global Initiative for Asthma (GINA) 2014) guidelines.

TABLE 1
Clinical characteristics of asthmatic patients (single agent study).

| Age | Gender | Smoker | Pack/year | FEV1% (pre) | FEV1% (post) |
|---|---|---|---|---|---|
| 56 | F | Ex | 40 | 23 | 33 |
| 48 | F | No | — | 38 | 48 |
| 63 | M | No | — | 70 | 88 |
| 62 | F | Yes | 30 | 85 | 96 |
| 62 | M | Ex | 50 | 51 | 70 |

Ex: quit smoking

TABLE 2
Clinical characteristics of asthmatic patients (Compound
A in combination with a corticosteroid).

| Age | Gender | Smoker | Pack/year | FEV1% (pre) | FEV1% (post) |
|---|---|---|---|---|---|
| 49 | M | Ex | 20 | 36 | 43 |
| 60 | M | No | — | 30 | 39 |
| 67 | F | No | — | 34 | 44 |
| 54 | F | No | — | 57 | 69 |
| 59 | F | Ex | 35 | 70 | 79 |

Ex: quit smoking

Mononuclear cells were isolated from peripheral blood of healthy volunteers, and asthmatic patients. Briefly, PBMC were isolated from peripheral venous blood by standard laboratory procedures. Peripheral venous blood was mixed with dextran 500 at 3% (in 0.9% saline) in a proportion of 2:1. This mixture was incubated at room temperature for 30 min until sedimentation of erythrocytes. The upper phase was carefully collected and layered on Ficoll-Paque Histopaque 1077 density gradient in a proportion of 3:1. (PBMC) layer was isolated and quantified.

For the single agent study, isolated mononuclear cells were incubated with Compound A, or vehicle for 30 minutes before incubation with or without LPS for 6 hours in standard cell culture conditions (37° C. and 5% CO₂). For the combination experiments, the same procedure was followed except that cells were incubated with Compound A (1 μM) in combination with fluticasone (0.1 nM). Supernatants were collected to measure IL-1β, IL-6, and GM-CSF. Data was analysed using Graphpad Prism.

### Results

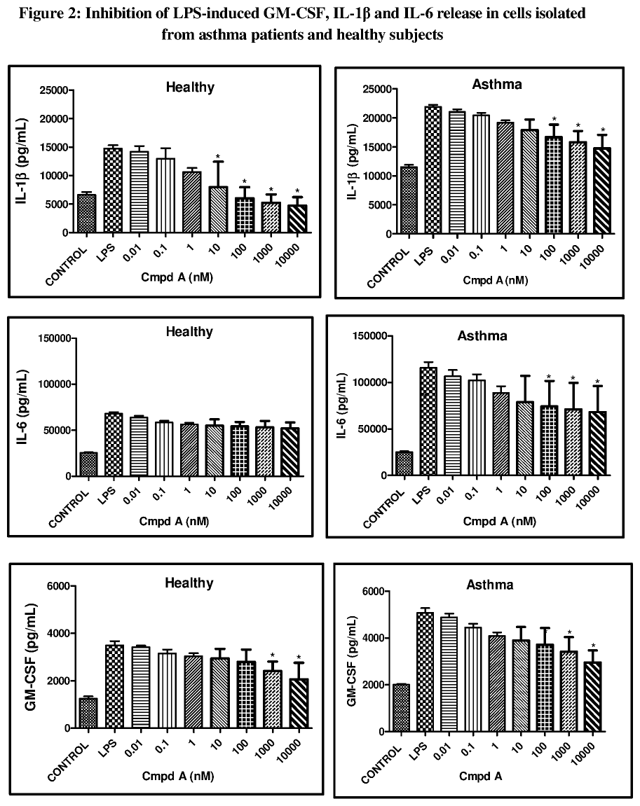

In asthmatic patients, Compound A inhibited GM-CSF, IL6 and IL1β release induced by LPS stimulus (FIG. 2), reaching a percentage maximum effect (% Emax) of 80±16.4%, 52.5±30.3% and 68.9±23.7%, respectively. In healthy donors, Compound A showed higher % Emax for IL1β confirming results observed in asthmatic mononuclear cells.

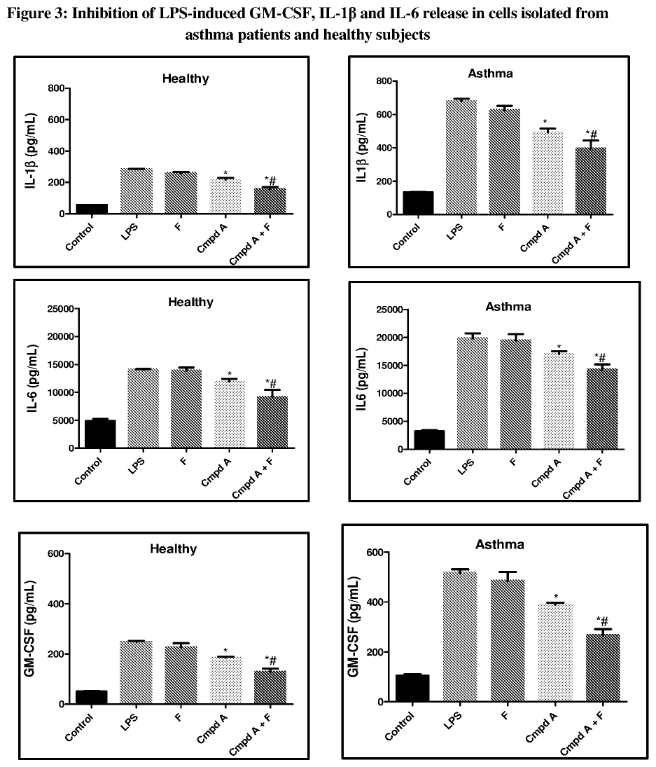

Compound A in combination with fluticasone inhibited LPS-induced GM-CSF, IL-1β and IL-6 release in cells isolated from asthma patients and healthy subjects (FIG. 3).

### CONCLUSION

Inhibitory effect of combination of Compound A with fluticasone was significantly better as compared to Compound A alone.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as described above. It is intended that the appended claims define the scope of the invention and that methods and structures within the scope of these claims and their equivalents be covered thereby.

All publications, patents and patent applications cited in this application are herein incorporated by reference to the same extent as if each individual publication, patent or patent application was specifically and individually indicated to be incorporated herein by reference.

We claim:

1. A method of treating an autoimmune, respiratory and/or inflammatory disease or condition, the method comprising administering to a subject in need thereof a therapeutically effective amount of (i) a CRAC modulator, and (ii) a corticosteroid.

2. The method according to claim 1, wherein the CRAC modulator is a CRAC inhibitor.

3. The method according to claim 1, wherein the CRAC modulator is
(i) a compound of formula (I) 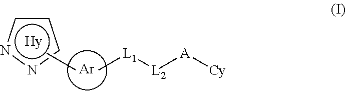
or a tautomer, N-oxide, pharmaceutically acceptable ester or pharmaceutically acceptable salt thereof, wherein
Ring Hy represents 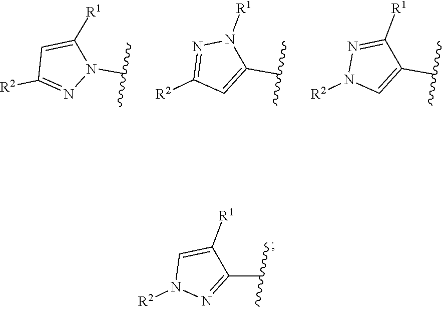
Ring Hy is optionally substituted with R′″;
R¹ and R² are the same or different and are selected from CH₃, CH₂F, CHF₂, CF₃, substituted or unsubstituted C₍₃₋₅₎cycloalkyl, CH₂—OR^{a}, CH₂—NR^{a}R^{b} and COOH;
Ring Ar represents: 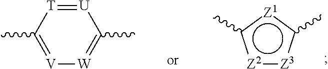
T, U, V and W are the same or different and are independently selected from CR^{a} and N;
Z¹, Z² and Z³ are the same or different and are selected from CR^{a}, CR^{a}R^{b}, O, S and —NR^{a}, with the proviso that at least one of Z¹, Z² and Z³ represents O, S or —NR^{a};
L₁ and L₂ together represent —NH—C(═X)—, —NH—S(═O)_{q}—, —C(═X)NH—, —NH—CR′R″— or —S(═O)_{q}NH—;
A is absent or selected from —(CR′R″)—, O, S(═O)_{q}, C(═X) and —NR^{a};
each occurrence of R′ and R″ are the same or different and are selected from hydrogen, hydroxy, cyano, halogen, —OR^{a}, —COOR^{a}, —S(═O)_{q}—R^{a}, —NR^{a}R^{b}, —C(═X)—R^{a}, substituted or unsubstituted C₍₁₋₆₎ alkyl group, substituted or unsubstituted C₍₁₋₆₎ alkenyl, substituted or unsubstituted C₍₁₋₆₎ alkynyl, and substituted or unsubstituted C₍₃₋₅₎ cycloalkyl, or R′ and R″ together with the common atom to which they are attached may be joined to form a saturated 3-6 member carbocyclic ring; which may optionally include one or more heteroatoms which may be same or different and are selected from O, NR^{a} and S;
R′″ is selected from hydrogen, hydroxy, cyano, halogen, —OR^{a}, —COOR^{a}, —S(═O)_{q}—R^{a}, —NR^{a}R^{b}, —C(═X)—R^{a}, substituted or unsubstituted C₍₁₋₆₎ alkyl group, substituted or unsubstituted C₍₁₋₆₎ alkenyl, substituted or unsubstituted C₍₁₋₆₎ alkynyl, and substituted or unsubstituted C₍₃₋₅₎cycloalkyl;
each occurrence of X is independently selected from O, S and —NR^{a};
Cy is selected from substituted or unsubstituted cycloalkyl group, substituted or unsubstituted heterocyclyl, substituted or unsubstituted aryl, and substituted or unsubstituted heteroaryl;
each occurrence of R^{a} and R^{b} are the same or different and are selected from hydrogen, nitro, hydroxy, cyano, halogen, —OR^{c}, —S(═O)_{q}—R^{c}, —C(═Y)—R^{c}, —CR^{c}R^{d}—C(═Y)—R^{c}, —CR^{c}R^{d}—Y—CR^{c}R^{d}—, —C(═Y)—NR^{c}R^{d}—, —NRR^{d}—C(═Y)—NR^{c}R^{d}—, —S(═O)_{q}—NR^{c}R^{d}—, —NR^{c}R^{d}—S(═O)_{q}—NR^{c}R^{d}—, —NR^{c}R^{d}—NR^{c}R^{d}—, substituted or unsubstituted alkyl, substituted or unsubstituted alkenyl, substituted or unsubstituted alkynyl, optionally substituted or unsubstituted cycloalkyl, substituted or unsubstituted cycloalkylakyl, substituted or unsubstituted cycloalkenyl, substituted or unsubstituted heterocylyl, substituted or unsubstituted heterocyclylalkyl, substituted or unsubstituted aryl, substituted or unsubstituted arylalkyl, substituted or unsubstituted heteroaryl, and substituted or unsubstituted heteroarylalkyl, or when R^{a} and R^{b} are directly bound to the same atom, they may be joined to form a substituted or unsubstituted saturated or unsaturated 3-10 member ring, which may optionally include one or more heteroatoms which may be the same or different and are selected from O, NR^{c} and S;
each occurrence of R^{c} and R^{d} may be same or different and are selected from hydrogen, nitro, hydroxy, cyano, halogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted heterocyclic group, substituted or unsubstituted heterocyclylalkyl, or when two R^{c} and/or R^{d} substituents are directly bound to the same atom, they may be joined to form a substituted or unsubstituted saturated or unsaturated 3-10 member ring, which may optionally include one or more heteroatoms which are the same or different and are selected from O, NH and S;
each occurrence of Y is independently selected from O, S and —NR^{a}; and
each occurrence of q independently represents 0, 1 or 2; or
(ii)
CM2489;
CM4620;
N-(5-(6-chloro-2,2-difluorobenzo[d][1,3]dioxol-5-yl)pyrazin-2-yl)-2-fluoro-6-methylbenzamide;
N-[4-[3,5-Bis(trifluoromethyl)-1H-pyrazol-1-yl]phenyl]-4-methyl-1,2,3-thiadiazole-5-carboxamide (YM-58483);
2,6-Difluoro-N-{5-[4-methyl-1-(5-methyl-thiazol-2-yl)-1,2,5,6-tetrahydro-pyridin-3-yl]-pyrazin-2-yl}-benzamid (R02959);
2,6-Difluoro-N-(1-(4-hydroxy-2-(trifluoromethyl)benzyl)-1H-pyrazol-3-yl)benzamide (GSK-7975A);
2,6-Difluoro-N-(1-(2-phenoxybenzyl)-1H-pyrazol-3-yl)benzamide (GSK5503A);
N-(2′,5′-Dimethoxy[1,1′-biphenyl]-4-yl)-3-fluoro-4-pyridinecarboxamide (Synta 66), or a pharmaceutically acceptable salt thereof.

4. The method according to any one of claims 1-3, wherein the CRAC modulator is a compound of formula (IA) 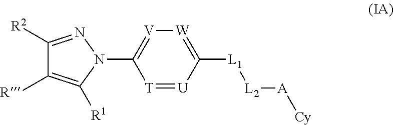
or a tautomer, N-oxide, pharmaceutically acceptable ester, or pharmaceutically acceptable salt thereof, wherein
both R¹ and R² are cyclopropyl or one of R¹ and R² is CF₃ and the other is cyclopropyl;
T is CF or N and U, V, W are independently CH, CF or N;
L₁ and L₂ together represent —NH—C(═X)—, —NH—S(═O)_{q}—, —C(═X)NH—, or —S(═O)_{q}NH— or —NH—CR′R″—;
A is absent or selected from —(CR′R″)— and —NR^{a};
each occurrence of R′ and R″ are the same or different and are independently selected from hydrogen or substituted or unsubstituted C₍₁₋₆₎ alkyl group or R′ and R″ may be joined to form a substituted or unsubstituted saturated or unsaturated 3-6 membered ring, which may optionally include one or more heteroatoms which may be same or different and are selected from O, NR^{a} and S;
R′″ is selected from hydrogen or halogen;
each occurrence of X is independently selected from O, S and —NR^{a};
Cy is selected from 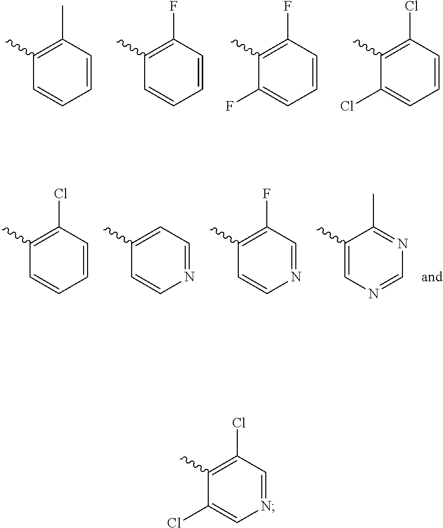
each occurrence of R^{a} is independently selected from hydrogen, nitro, hydroxy, cyano, halogen, —OR^{c}, —S(═O)_{q}—R^{c}, —NR^{c}R^{d}, —C(═Y)—R^{c}, —CR^{c}R^{d}—C(═Y)—R^{c}, —CR^{c}R^{d}—Y—CR^{c}R^{d}—, —C(═Y)—NR^{c}R^{d}—, —NRR^{d}—C(═Y)—NR^{c}R^{d}—, —S(═O)_{q}—NR^{c}R^{d}—, —NR^{c}R^{d}—S(═O)_{q}—NR^{c}R^{d}—, —NR^{c}R^{d}—NR^{c}R^{d}—, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted heterocylyl, substituted or unsubstituted heterocyclylalkyl, substituted or unsubstituted aryl, substituted or unsubstituted arylalkyl, substituted or unsubstituted heteroaryl, and substituted or unsubstituted heteroarylalkyl;
each occurrence of R^{c} and R^{d} may be same or different and are independently selected from hydrogen, nitro, hydroxy, cyano, halogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted heterocyclic group, substituted or unsubstituted heterocyclylalkyl, or when two R^{c} and/or R^{d} substitutents are directly bound to the same atom, they may be joined to form a substituted or unsubstituted saturated or unsaturated 3-10 member ring, which may optionally include one or more heteroatoms which are the same or different and are selected from O, NH and S; each occurrence of Y is independently selected from O, S and —NR^{a}; and
each occurrence of q independently represents 0, 1 or 2.

5. The method according to any one of claims 1-3, wherein the CRAC modulator is a compound of formula (IB) 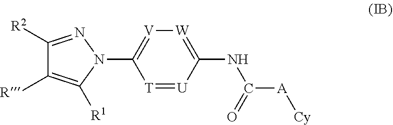
or a tautomer, N-oxide, pharmaceutically acceptable ester or pharmaceutically acceptable salt thereof, wherein
R¹ and R² are both cyclopropyl or one of R¹ and R² is CF₃ and the other is cyclopropyl;
R′″ is selected from hydrogen, hydroxy, cyano, halogen, —OR^{a}, —COOR^{a}, —S(═O)_{q}—R^{a}, —NR^{a}R^{b}, —C(═X)—R^{a}, substituted or unsubstituted C₍₁₋₆₎ alkyl group, substituted or unsubstituted C₍₁₋₆₎ alkenyl, substituted or unsubstituted C₍₁₋₆₎ alkynyl, and substituted or unsubstituted C₍₃₋₅₎cycloalkyl;
T, U, V and W are the same or different and are independently selected from CR^{a} and N;
A is absent or is selected from —CH₂—, —CHMe-, 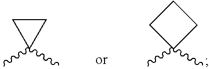
Cy is a bicyclic ring selected from substituted or unsubstituted cycloalkyl group, substituted or unsubstituted heterocyclyl, substituted or unsubstituted aryl, and substituted or unsubstituted heteroaryl;
each occurrence of R^{a} and R^{b} are the same or different and are independently selected from hydrogen, nitro, hydroxy, cyano, halogen, —OR^{c}, —S(═O)_{q}—R^{c}, —NR^{c}R^{d}, —C(═Y)—R^{c}, —CR^{c}R^{d}—C(═Y)—R^{c}, —CR^{c}R^{d}—Y—CR^{c}R^{d}—, —C(═Y)—NR^{c}R^{d}—, —NRR^{d}—C(═Y)—NR^{c}R^{d}—, —S(═O)_{q}—NR^{c}R^{d}—, —NR^{c}R^{d}—S(═O)_{q}—NR^{c}R^{d}—, —NR^{c}R^{d}—NR^{c}R^{d}—, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted heterocylyl, substituted or unsubstituted heterocyclylalkyl, substituted or unsubstituted aryl, substituted or unsubstituted arylalkyl, substituted or unsubstituted heteroaryl, and substituted or unsubstituted heteroarylalkyl, or when R^{a} and R^{b} are directly bound to the same atom, they may be joined to form a substituted or unsubstituted saturated or unsaturated 3-10 member ring, which may optionally include one or more heteroatoms which may be the same or different and are selected from O, NR^{c} and S;
each occurrence of R^{c} and R^{d} may be same or different and are independently selected from hydrogen, nitro, hydroxy, cyano, halogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted heterocyclic group, substituted or unsubstituted heterocyclylalkyl, or when two R^{c} and/or R^{d} substitutents are directly bound to the same atom, they may be joined to form a substituted or unsubstituted saturated or unsaturated 3-10 member ring, which may optionally include one or more heteroatoms which are the same or different and are selected from O, NH and S;
each occurrence of X is independently selected from O, S and —NR^{a};
each occurrence of Y is independently selected from O, S and —NR^{a}; and
each occurrence of q independently represents 0, 1 or 2;

6. The method according to any one of claims 1-5, wherein R¹ and R² are both cyclopropyl or one of R¹ and R² is CF₃ and the other is cyclopropyl

7. The method according to any one of claims 1-6, wherein Hy is

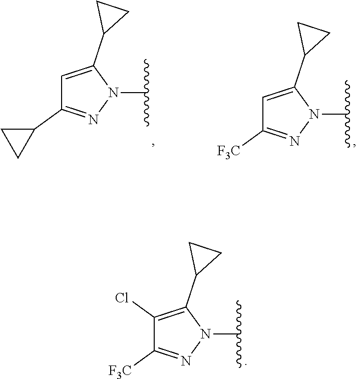 [SMILES: *n1nc(C2CC2)cc1C1CC1 ; *n1nc(C(F)(F)F)cc1C1CC1 ; *n1nc(C(F)(F)F)c(Cl)c1C1CC1]

8. The method according to any one of claims 1-7, wherein Ring Ar is selected from

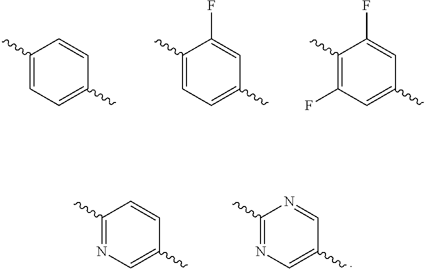

9. The method according to any one of claims 1-8, wherein L₁ and L₂ together represent —NH—C(═X)— or —C(═X)—NH—.

10. The method according to any one of claims 1-9, wherein Cy is selected from

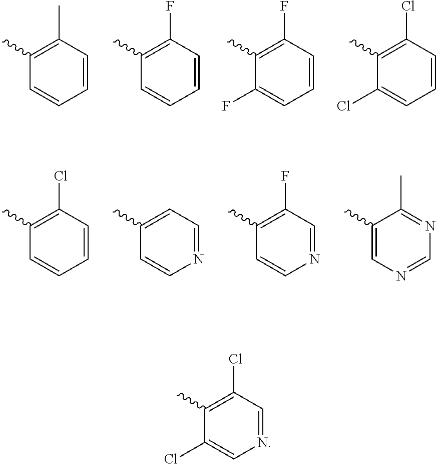

11. The method according to any one of claims 1-10, wherein Cy is selected from

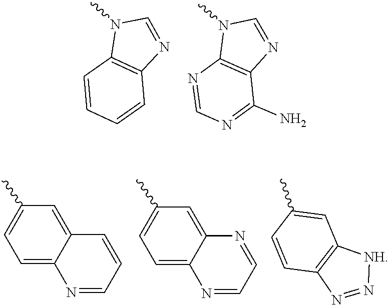

12. The method according to any one of claims 1-11, wherein the CRAC modulator is selected from
N-[4-(3,5-dicyclopropyl-1H-pyrazol-1-yl)phenyl]-4-methyl-1,2,3-thiadiazole-5-carboxamide
N-[4-(3,5-dicyclopropyl-1H-pyrazol-1-yl)phenyl]-4-methylthiazole-5-carboxamide
N-[4-(3,5-dicyclopropyl-1H-pyrazol-1-yl)phenyl]-2,4-dimethylthiazole-5-carboxamide
N-[4-(3,5-dicyclopropyl-1H-pyrazol-1-yl)phenyl]-5-methylisoxazole-4-carboxamide
N-[4-(3,5-dicyclopropyl-1H-pyrazol-1-yl)phenyl]-3,5-dimethylisoxazole-4-carboxamide
N-[4-(3,5-dicyclopropyl-1H-pyrazol-1-yl)phenyl]benzamide
N-[4-(3,5-dicyclopropyl-1H-pyrazol-1-yl)phenyl]-2-methylbenzamide
N-[4-(3,5-dicyclopropyl-1H-pyrazol-1-yl)phenyl]-2,6-difluorobenzamide
N-[4-(3,5-dicyclopropyl-1H-pyrazol-1-yl)phenyl]-2,3-difluorobenzamide
N-[4-(3,5-dicyclopropyl-1H-pyrazol-1-yl)phenyl-3-(methylsulfonyl)benzamide
N-[4-(3,5-dicyclopropyl-1H-pyrazol-1-yl)phenyl]-4-(methylsulfonyl)benzamide
2-chloro-N-[4-(3,5-dicyclopropyl-1H-pyrazol-1-yl)phenyl]-5-(methylthio)benzamide
2-chloro-N-[4-(3,5-dicyclopropyl-1H-pyrazol-1-yl)phenyl)-5-(methylsulfonyl)benzamide
N-[4-(3,5-dicyclopropyl-1H-pyrazol-1-yl)phenyl]nicotinamide hydrochloride
N-[4-(3,5-dicyclopropyl-1H-pyrazol-1-yl)phenyl]isonicotinamide hydrochloride
N-[4-(3,5-dicyclopropyl-1H-pyrazol-1-yl)phenyl]-3-fluoroisonicotinamide
3,5-dichloro-N-(4-(3,5-dicyclopropyl-1H-pyrazol-1-yl)phenyl)isonicotinamide
N-[4-(3,5-dicyclopropyl-1H-pyrazol-1-yl)phenyl]-4-methylpyrimidine-5-carboxamide
N-[4-(3,5-dicyclopropyl-1H-pyrazol-1-yl)phenyl]-2-phenylacetamide
N-[4-(3,5-dicyclopropyl-1H-pyrazol-1-yl)phenyl]-2-(4-fluorophenyl)acetamide
N-[4-(3,5-dicyclopropyl-1H-pyrazol-1-yl)phenyl]-1-phenylcyclopropanecarboxamide
N-[4-(3,5-dicyclopropyl-1H-pyrazol-1-yl)phenyl]-2-(pyridin-2-yl)acetamide
N-[4-(3,5-dicyclopropyl-1H-pyrazol-1-yl)phenyl]-2-(pyridin-3-yl)acetamide
N-[4-(3,5-dicyclopropyl-1H-pyrazol-1-yl)phenyl]-2-(pyridin-4-yl)acetamide
N-[4-(3,5-dicyclopropyl-1H-pyrazol-1-yl)phenyl]-2-(piperazin-1-yl)acetamide
N-[4-(3,5-dicyclopropyl-1H-pyrazol-1-yl)phenyl]-2-morpholinoacetamide
N-[4-(3,5-dicyclopropyl-1H-pyrazol-1-yl)phenyl]benzenesulfonamide
N-[4-(3,5-dicyclopropyl-1H-pyrazol-1-yl)-3-fluorophenyl]-4-methyl-1,2,3-thiadiazole-5-carboxamide
N-[4-(3,5-dicyclopropyl-1H-pyrazol-1-yl)-3-fluorophenyl]-4-methylthiazole-5-carboxamide
N-[4-(3,5-dicyclopropyl-1H-pyrazol-1-yl)-3-fluorophenyl]-3,5-dimethylisoxazole-4-carboxamide
N-[4-(3,5-dicyclopropyl-1H-pyrazol-1-yl)-3-fluorophenyl]-2methyl benzamide
N-[4-(3,5-dicyclopropyl-1H-pyrazol-1-yl)-3-fluorophenyl]-2,3-difluorobenzamide
N-[4-(3,5-dicyclopropyl-1H-pyrazol-1-yl)-3-fluorophenyl]-2,6-difluorobenzamide
N-[4-(3,5-dicyclopropyl-1H-pyrazol-1-yl)-3-fluorophenyl]nicotinamide
N-[4-(3,5-dicyclopropyl-1H-pyrazol-1-yl)-3-fluorophenyl]isonicotinamide
N-[4-(3,5-dicyclopropyl-1H-pyrazol-1-yl)-3-fluorophenyl]-4-methylpyrimidine-5-carboxamide
N-[4-(4-chloro-3,5-dicyclopropyl-1H-pyrazol-1-yl)-3-fluorophenyl]-4-methyl-1,2,3-thiadiazole-5-carboxamide
N-[6-(3,5-dicyclopropyl-1H-pyrazol-1-yl)pyridin-3-yl]-4-methyl-1,2,3-thiadiazole-5-carboxamide
N-[6-(3,5-dicyclopropyl-1H-pyrazol-1-yl)pyridin-3-yl]-4-methylthiazole-5-carboxamide
N-[6-(3,5-dicyclopropyl-1H-pyrazol-1-yl)pyridin-3-yl]-2,4-dimethylthiazole-5-carboxamide
N-[6-(3,5-dicyclopropyl-1H-pyrazol-1-yl)pyridin-3-yl]-3,5-dimethylisoxazole-4-carboxamide
6-(3,5-dicyclopropyl-1H-pyrazol-1-yl)-N-o-tolylnicotinamide
N-[6-(3,5-dicyclopropyl-1H-pyrazol-1-yl)pyridin-3-yl]-2-fluorobenzamide
N-[6-(3,5-dicyclopropyl-1H-pyrazol-1-yl)pyridin-3-yl]-2,3-difluorobenzamide
N-[6-(3,5-dicyclopropyl-1H-pyrazol-1-yl)pyridin-3-yl]-2,6-difluorobenzamide
N-[6-(3,5-dicyclopropyl-1H-pyrazol-1-yl)pyridin-3-yl]nicotinamide dihydrochloride
N-[6-(3,5-dicyclopropyl-1H-pyrazol-1-yl)pyridin-3-yl]isonicotinamide
N-[6-(3,5-dicyclopropyl-1H-pyrazol-1-yl)pyridin-3-yl]-3-fluoroisonicotinamide
3,5-dichloro-N-{4-[5-cyclopropyl-3-(trifluoromethyl)-1H-pyrazol-1-yl]phenyl}isonicotinamide
3,5-dichloro-N-[6-(3,5-dicyclopropyl-1H-pyrazol-1-yl)pyridin-3-yl]isonicotinamide
N-[6-(3,5-dicyclopropyl-1H-pyrazol-1-yl)pyridin-3-yl]-4-methylpyrimidine-5-carboxamide
N-{4-[5-cyclopropyl-3-(trifluoromethyl)-1H-pyrazol-1-yl]phenyl}-4-methyl-1,2,3-thiadiazole-5-carboxamide
N-{4-[5-cyclopropyl-3-(trifluoromethyl)-1H-pyrazol-1-yl]phenyl}-4-methylthiazole-5-carboxamide
N-{4-[5-cyclopropyl-3-(trifluoromethyl)-1H-pyrazol-1-yl]phenyl}-N,4-dimethylthiazole-5-carboxamide
N-{4-[5-cyclopropyl-3-(trifluoromethyl)-1H-pyrazol-1-yl]phenyl}-2,4-dimethylthiazole-5-carboxamide
N-{4-[5-cyclopropyl-3-(trifluoromethyl)-1H-pyrazol-1-yl]phenyl}-5-methylisoxazole-4-carboxamide
N-{4-[5-cyclopropyl-3-(trifluoromethyl)-1H-pyrazol-1-yl]phenyl}-3,5-dimethylisoxazole-4-carboxamide
N-{4-[5-cyclopropyl-3-(trifluoromethyl)-1H-pyrazol-1-yl]phenyl}-1-methyl-1H-imidazole-2-carboxamide
N-{4-[3-cyclopropyl-5-(trifluoromethyl)-1H-pyrazol-1-yl]phenyl}-4-methyl-1H-imidazole-5-carboxamide
N-{4-[5-cyclopropyl-3-(trifluoromethyl)-1H-pyrazol-1-yl]phenyl}-2-methylbenzamide
N-{4-[5-cyclopropyl-3-(trifluoromethyl)-1H-pyrazol-1-yl]phenyl}-2,3-difluorobenzamide
N-{4-[5-cyclopropyl-3-(trifluoromethyl)-1H-pyrazol-1-yl]phenyl}-2,6-difluorobenzamide
N-{4-[5-cyclopropyl-3-(trifluoromethyl)-1H-pyrazol-1-yl]phenyl}-3-(methylsulfonyl) benzamide
2-chloro-N-{4-[5-cyclopropyl-3-(trifluoromethyl)-1H-pyrazol-1-yl]phenyl}-5-(methylthio) benzamide
2-chloro-N-{4-[5-cyclopropyl-3-(trifluoromethyl)-1H-pyrazol-1-yl]phenyl}-5-(methylsulfonyl)benzamide
N-{4-[5-cyclopropyl-3-(trifluoromethyl)-1H-pyrazol-1-yl]phenyl}pyridine-4-carboxamide hydrochloride
N-{4-[5-cyclopropyl-3-(trifluoromethyl)-1H-pyrazol-1-yl]phenyl}-3-fluoro isonicotinamide
N-{4-[5-cyclopropyl-3-(trifluoromethyl)-1H-pyrazol-1-yl]phenyl}-4-methylpyrimidine-5-carboxamide
N-{4-[5-cyclopropyl-3-(trifluoromethyl)-1H-pyrazol-1-yl]phenyl}-2,4-dimethyl pyrimidine-5-carboxamide
N-{4-[5-cyclopropyl-3-(trifluoromethyl)-1H-pyrazol-1-yl]phenyl}-2-(4-fluorophenyl)acetamide
N-{4-[5-cyclopropyl-3-(trifluoromethyl)-1H-pyrazol-1-yl]phenyl}-2-(pyridin-2-yl)acetamide
N-{4-[5-cyclopropyl-3-(trifluoromethyl)-1H-pyrazol-1-yl]phenyl}-2-(pyridin-3-yl)acetamide
N-{4-[5-cyclopropyl-3-(trifluoromethyl)-1H-pyrazol-1-yl]phenyl}-2-(pyridin-4-yl)acetamide
4-[5-cyclopropyl-3-(trifluoromethyl)-1H-pyrazol-1-yl]-N-[(4-methylthiazol-5-yl)methyl]aniline
1-{4-[5-cyclopropyl-3-(trifluoromethyl)-1H-pyrazol-1-yl]phenyl}-3-(4-methyl-1,2,3-thiadiazol-5-yl)urea
1-{4-[5-cyclopropyl-3-(trifluoromethyl)-1H-pyrazol-1-yl]phenyl}-3-(4-methylthiazol-5-yl)urea
1-{4-[5-cyclopropyl-3-(trifluoromethyl)-1H-pyrazol-1-yl]phenyl}-3-(4-methylpyrimidin-5-yl)urea
4-[5-cyclopropyl-3-(trifluoromethyl)-1H-pyrazol-1-yl]-N-(4-methylthiazol-5-yl) benzamide
4-[5-cyclopropyl-3-(trifluoromethyl)-1H-pyrazol-1-yl]-N-(2,6-difluorophenyl) benzamide
N-{4-[4-chloro-5-cyclopropyl-3-(trifluoromethyl)-1H-pyrazol-1-yl]phenyl}-4-methylthiazole-5-carboxamide
N-{4-[4-chloro-5-cyclopropyl-3-(trifluoromethyl)-1H-pyrazol-1-yl]phenyl}-2-(pyridin-2-yl)acetamide
N-{4-[5-cyclopropyl-3-(trifluoromethyl)-1H-pyrazol-1-yl]-3-fluorophenyl}-4-methyl-1,2,3-thiadiazole-5-carboxamide
N-{4-[5-cyclopropyl-3-(trifluoromethyl)-1H-pyrazol-1-yl]-3-fluorophenyl}-4-methylthiazole-5-carboxamide
N-{4-[5-cyclopropyl-3-(trifluoromethyl)-1H-pyrazol-1-yl]-3-fluorophenyl}-5-methylisoxazole-4-carboxamide
N-{4-[5-cyclopropyl-3-(trifluoromethyl)-1H-pyrazol-1-yl]-3-fluorophenyl}-3,5-dimethylisoxazole-4-carboxamide
N-{4-[5-cyclopropyl-3-(trifluoromethyl)-1H-pyrazol-1-yl]-3-fluorophenyl}-2-methylbenzamide
N-{4-[5-cyclopropyl-3-(trifluoromethyl)-1H-pyrazol-1-yl]-3-fluorophenyl}-2,3-difluorobenzamide
N-{4-[5-cyclopropyl-3-(trifluoromethyl)-1H-pyrazol-1-yl]-3-fluorophenyl}-2,6-difluorobenzamide
N-{4-[5-cyclopropyl-3-(trifluoromethyl)-1H-pyrazol-1-yl]-3-fluorophenyl}nicotinamide
N-{4-[5-cyclopropyl-3-(trifluoromethyl)-1H-pyrazol-1-yl]-3-fluorophenyl}isonicotinamide
N-{4-[5-cyclopropyl-3-(trifluoromethyl)-1H-pyrazol-1-yl]-3-fluorophenyl}-3-fluoroisonicotinamide
3,5-dichloro-N-{4-[5-cyclopropyl-3-(trifluoromethyl)-1H-pyrazol-1-yl]-3-fluorophenyl}isonicotinamide
N-{4-[5-cyclopropyl-3-(trifluoromethyl)-1H-pyrazol-1-yl]-3-fluorophenyl}-4-methylpyrimidine-5-carboxamide
N-{4-[5-cyclopropyl-3-(trifluoromethyl)-1H-pyrazol-1-yl]-3-fluorophenyl}-N,4-dimethylpyrimidine-5-carboxamide
N-{4-[4-chloro-5-cyclopropyl-3-(trifluoromethyl)-1H-pyrazol-1-yl]-3-fluorophenyl}-4-methyl-1,2,3-thiadiazole-5-carboxamide
N-{4-[4-chloro-5-cyclopropyl-3-(trifluoromethyl)-1H-pyrazol-1-yl]-3-fluorophenyl}-2-(pyridin-2-yl)acetamide
1-{4-[5-cyclopropyl-3-(trifluoromethyl)-1H-pyrazol-1-yl]-3-fluorophenyl}-3-(4-methylpyrimidin-5-yl)urea
N-{4-[5)-cyclopropyl-3-(trifluromethyl)-1H-pyrazol-1-yl]3-fluorophenyl}-2,6-dichloro benzamide
4-[5-cyclopropyl-3-(trifluoromethyl)-1H-pyrazol-1-yl]-N-(2,3-difluorophenyl)-3-fluorobenzamide
4-[5-cyclopropyl-3-(trifluoromethyl)-1H-pyrazol-1-yl]-N-(2,6-difluorophenyl)-3-fluorobenzamide
N-{6-[5-cyclopropyl-3-(trifluoromethyl)-1H-pyrazol-1-yl]pyridin-3-yl}-4-methyl-1,2,3-thiadiazole-5-carboxamide
N-{6-[5-cyclopropyl-3-(trifluoromethyl)-1H-pyrazol-1-yl]pyridin-3-yl}-4-methylthiazole-5-carboxamide
N-{6-[5-cyclopropyl-3-(trifluoromethyl)-1H-pyrazol-1-yl]pyridin-3-yl}-3,5-dimethylisoxazole-4-carboxamide
N-{6-[5-cyclopropyl-3-(trifluoromethyl)-1H-pyrazol-1-yl]pyridin-3-yl}-2-methylbenzamide
2-chloro-N-{6-[5-cyclopropyl-3-(trifluoromethyl)-1H-pyrazol-1-yl]pyridin-3-yl}benzamide
N-(6-(5-cyclopropyl-3-(trifluoromethyl)-1H-pyrazol-1-yl)pyridin-3-yl)-2-fluorobenzamide
N-{6-[5-cyclopropyl-3-(trifluoromethyl)-1H-pyrazol-1-yl]pyridin-3-yl}-2,3-difluorobenzamide
N-{6-[5-cyclopropyl-3-(trifluoromethyl)-1H-pyrazol-1-yl]pyridin-3-yl}-2,6-difluorobenzamide
N-{6-[5-cyclopropyl-3-(trifluoromethyl)-1H-pyrazol-1-yl]pyridin-3-yl}picolinamide
N-{6-[5-cyclopropyl-3-(trifluoromethyl)-1H-pyrazol-1-yl]pyridin-3-yl}-3-methylpicolinamide
N-{6-[5-cyclopropyl-3-(trifluoromethyl)-1H-pyrazol-1-yl]pyridin-3-yl}nicotinamide
N-{6-[5-cyclopropyl-3-(trifluoromethyl)-1H-pyrazol-1-yl]pyridin-3-yl}-2-methylnicotinamide
N-{6-[5-cyclopropyl-3-(trifluoromethyl)-1H-pyrazol-1-yl]pyridin-3-yl}isonicotinamide
N-{6-[5-cyclopropyl-3-(trifluoromethyl)-1H-pyrazol-1-yl]pyridin-3-yl}-3-fluoroisonicotinamide
3,5-dichloro-N-{6-[5-cyclopropyl-3-(trifluoromethyl)-1H-pyrazol-1-yl]pyridin-3-yl}isonicotinamide
N-{6-[5-cyclopropyl-3-(trifluoromethyl)-1H-pyrazol-1-yl]pyridin-3-yl}-4-methylpyrimidine-5-carboxamide
N-{6-[5-cyclopropyl-3-(trifluoromethyl)-1H-pyrazol-1-yl]pyridin-3-yl}-2-(pyridin-2-yl)acetamide
N-{6-[5-cyclopropyl-3-(trifluoromethyl)-1H-pyrazol-1-yl]pyridin-3-yl}-2-(pyridin-4-yl)acetamide
N-{4-[4-chloro-5-cyclopropyl-3-(trifluoromethyl)-1H-pyrazol-1-yl]-3-fluorophenyl}-4-methylpyrimidine-5-carboxamide
1-{6-[3-cyclopropyl-5-(trifluoromethyl)-1H-pyrazol-1-yl]pyridin-3-yl}-3-(4-methylthiazol-5-yl)urea
6-[5-cyclopropyl-3-(trifluoromethyl)-1H-pyrazol-1-yl]-N-(2,3-difluorophenyl) nicotinamide
6-[5-cyclopropyl-3-(trifluoromethyl)-1H-pyrazol-1-yl]-N-(2,6-difluorophenyl) nicotinamide
N-{6-[4-chloro-5-cyclopropyl-3-(trifluoromethyl)-1H-pyrazol-1-yl]pyridin-3-yl}-4-methylthiazole-5-carboxamide
N-{2-[5-cyclopropyl-3-(trifluoromethyl)-1H-pyrazol-1-yl]pyrimidin-5-yl}-2,6-difluorobenzamide
N-{4-[5-(fluoromethyl)-3-(trifluoromethyl)-1H-pyrazol-1-yl]phenyl}-4-methylthiazole-5-carboxamide
N-{4-[5-(difluoromethyl)-3-(trifluoromethyl)-1H-pyrazol-1-yl]phenyl}-4-methylthiazole-5-carboxamide
3,5-dichloro-N-[4-(3,5-dicyclopropyl-1H-pyrazol-1-yl)-3-fluorophenyl]isonicotinamide
N-(2-chloro-6-fluorophenyl)-4-[5-cyclopropyl-3-(trifluoromethyl)-1H-pyrazol-1-yl]-3-fluorobenzamide
N-{2-[5-cyclopropyl-3-(trifluoromethyl)-1H-pyrazol-1-yl]pyrimidin-5-yl}-4-methylthiazole-5-carboxamide
N-{4-[5-cyclopropyl-3-(trifluoromethyl)-1H-pyrazol-1-yl]-3,5-difluorophenyl}-4-methylpyrimidine-5-carboxamide
{4-[5-cyclopropyl-3-(trifluoromethyl)-1H-pyrazol-1-yl]-3-fluorophenyl}-1-phenylcyclobutanecarboxamide
N-{4-[5-cyclopropyl-3-(trifluoromethyl)-1H-pyrazol-1-yl]-3-fluorophenyl}-4-methyloxazole-5-carboxamide
N-{2-[5-cyclopropyl-3-(trifluoromethyl)-1H-pyrazol-1-yl]pyrimidin-5-yl}-4-methylpyrimidine-5-carboxamide
4-[5-cyclopropyl-3-(trifluoromethyl)-1H-pyrazol-1-yl]-3-fluoro-N-(4-methylpyrimidin-5-yl) benzamide and
N-{4-[3-cyclopropyl-5-(difluoromethyl)-1H-pyrazol-1-yl]-3-fluorophenyl}-2,6-difluorobenzamide;
N-{4-[5-cyclopropyl-3-(difluoromethyl)-1H-pyrazol-1-yl]-3-fluorophenyl}-2,6-difluorobenzamide
N-[4-(3,5-dicyclopropyl-1H-pyrazol-1-yl)phenyl]-1H-benzo[d]imidazole-6-carboxamide
N-[4-(3,5-dicyclopropyl-1H-pyrazol-1-yl)phenyl]-1H-benzo[d][1,2,3]triazole-6-carboxamide
N-[4-(3,5-dicyclopropyl-1H-pyrazol-1-yl)phenyl]quinoline-6-carboxamide hydrochloride
N-[4-(3,5-dicyclopropyl-1H-pyrazol-1-yl)phenyl]quinoxaline-6-carboxamide
2-(1H-benzo[d]imidazol-1-yl)-N-[4-(3,5-dicyclopropyl-1H-pyrazol-1-yl)phenyl]acetamide
2-(1H-benzo[d][1,2,3]triazol-1-yl)-N-[4-(3,5-dicyclopropyl-1H-pyrazol-1-yl)phenyl]acetamide
N-[4-(3,5-dicyclopropyl-1H-pyrazol-1-yl)phenyl]-2-(1H-indol-3-yl)acetamide
N-[4-(3,5-dicyclopropyl-1H-pyrazol-1-yl)phenyl]-2-(imidazo[1,2-a]pyridin-2-yl)acetamide hydrochloride
N-[4-(3,5-dicyclopropyl-1H-pyrazol-1-yl)phenyl]-2-(quinolin-6-yl)acetamide:
N-[4-(3,5-dicyclopropyl-1H-pyrazol-1-yl)phenyl]-2-(quinolin-6-yl)acetamide hydrochloride
2-(1H-benzo[d][1,2,3]triazol-1-yl)-N-(4-(3,5-dicyclopropyl-1H-pyrazol-1-yl)-3-fluorophenyl)acetamide
N-[4-(3,5-dicyclopropyl-1H-pyrazol-1-yl)-3-fluorophenyl]-2-(quinolin-6-yl)acetamide hydrochloride
N-[6-(3,5-dicyclopropyl-1H-pyrazol-1-yl)pyridin-3-yl]quinoline-6-carboxamide dihydrochloride
N-[6-(3,5-dicyclopropyl-1H-pyrazol-1-yl)pyridin-3-yl]quinoxaline-6-carboxamide
2-(1H-benzo[d][1,2,3]triazol-1-yl)-N-[6-(3,5-dicyclopropyl-1H-pyrazol-1-yl)pyridin-3-yl]acetamide
N-[6-(3,5-dicyclopropyl-1H-pyrazol-1-yl)pyridin-3-yl]-2-(quinolin-6-yl)acetamidedihydrochloride
N-{4-[5-cyclopropyl-3-(trifluoromethyl)-1H-pyrazol-1-yl]phenyl}quinoline-6-carboxamide hydrochloride
N-{4-[5-cyclopropyl-3-(trifluoromethyl)-1H-pyrazol-1-yl]phenyl}quinoxaline-6-carboxamide
2-(1H-benzo[d]imidazol-1-yl)-N-{4-[5-cyclopropyl-3-(trifluoromethyl)-1H-pyrazol-1-yl]phenyl}acetamide
2-(1H-benzo[d][1,2,3]triazol-1-yl)-N-{4-[5-cyclopropyl-3-(trifluoromethyl)-1H-pyrazol-1-yl]phenyl}acetamide
2-(2H-benzo[d][1,2,3]triazol-2-yl)-N-{4-[5-cyclopropyl-3-(trifluoromethyl)-1H-pyrazol-1-yl]phenyl}acetamide
2-(3H-[1,2,3]triazolo[4,5-b]pyridin-3-yl)-N-{4-[5-cyclopropyl-3-(trifluoromethyl)-1H-pyrazol-1-yl]phenyl}acetamide
(S)-2-(3H-[1,2,3]triazolo[4,5-b]pyridin-3-yl)-N-{4-[5-cyclopropyl-3-(trifluoromethyl)-1H-pyrazol-1-yl]phenyl}propanamide
2-(6-amino-9H-purin-9-yl)-N-{4-[5-cyclopropyl-3-(trifluoromethyl)-1H-pyrazol-1-yl]phenyl}acetamide
N-(4-(5-cyclopropyl-3-(trifluoromethyl)-1H-pyrazol-1-yl)phenyl)-2-(1,3-dimethyl-2,6-dioxo-2,3-dihydro-1H-purin-7(6H)-yl)acetamide
N-{4-[5-cyclopropyl-3-(trifluoromethyl)-1H-pyrazol-1-yl)phenyl)-2-(imidazo[1,2-a]pyridin-2-yl)acetamide hydrochloride
N-{4-[5-cyclopropyl-3-(trifluoromethyl)-1H-pyrazol-1-yl]phenyl}-2-(quinolin-6-yl)acetamide hydrochloride
N-{4-[5-cyclopropyl-3-(trifluoromethyl)-1H-pyrazol-1-yl]phenyl}-2-(quinolin-6-yl)propanamide hydrochloride
N-{4-[5-cyclopropyl-3-(trifluoromethyl)-1H-pyrazol-1-yl]-3-fluorophenyl}-1H-benzo[d][1,2,3]triazole-6-carboxamide
2-(1H-benzo[d][1,2,3]triazol-1-yl)-N-{4-[5-cyclopropyl-3-(trifluoromethyl)-1H-pyrazol-1-yl]-3-fluorophenyl}acetamide
N-{6-[5-cyclopropyl-3-(trifluoromethyl)-1H-pyrazol-1-yl]pyridin-3-yl}-1H-benzo[d][1,2,3]triazole-5-carboxamide
2-(1H-benzo[d][1,2,3]triazol-1-yl)-N-{6-[5-cyclopropyl-3-(trifluoromethyl)-1H-pyrazol-1-yl]pyridin-3-yl}acetamide
2-(2H-benzo[d][1,2,3]triazol-2-yl)-N-{6-[5-cyclopropyl-3-(trifluoromethyl)-1H-pyrazol-1-yl]pyridin-3-yl}acetamide
N-{6-[5-cyclopropyl-3-(trifluoromethyl)-1H-pyrazol-1-yl]pyridin-3-yl}-2-(quinolin-6-yl)acetamide hydrochloride
2-(1H-benzo[d][1,2,3]triazol-1-yl)-N-{6-[4-chloro-5-cyclopropyl-3-(trifluoromethyl)-1H-pyrazol-1-yl]pyridin-3-yl}acetamide
4-[5-cyclopropyl-3-(trifluoromethyl)-1H-pyrazol-1-yl]-3-fluoro-N-(quinolin-6-ylmethyl) benzamide hydrochloride and
1-[4-(3,5-dicyclopropyl-1H-pyrazol-1-yl)phenyl]-3-(quinolin-6-yl)urea,
and pharmaceutically acceptable salts thereof.

13. The method according to any one of claims 1-11, wherein the CRAC modulator is selected from
CM2489;
CM4620;
N-(5-(6-chloro-2,2-difluorobenzo[d][1,3]dioxol-5-yl)pyrazin-2-yl)-2-fluoro-6-methylbenzamide;
N-[4-[3,5-Bis(trifluoromethyl)-1H-pyrazol-1-yl]phenyl]-4-methyl-1,2,3-thiadiazole-5-carboxamide (YM-58483);
2,6-Difluoro-N-{5-[4-methyl-1-(5-methyl-thiazol-2-yl)-1,2,5,6-tetrahydro-pyridin-3-yl]-pyrazin-2-yl}-benzamid (R02959);
2,6-Difluoro-N-(1-(4-hydroxy-2-(trifluoromethyl)benzyl)-1H-pyrazol-3-yl)benzamide (GSK-7975A);
2,6-Difluoro-N-(1-(2-phenoxybenzyl)-1H-pyrazol-3-yl)benzamide (GSK5503A);
N-(2′,5′-Dimethoxy[1,1′-biphenyl]-4-yl)-3-fluoro-4-pyridinecarboxamide (Synta 66);
N-{6-[5-cyclopropyl-3-(trifluoromethyl)-1H-pyrazol-1-yl]pyridin-3-yl}-2-methylbenzamide;
N-{6-[5-cyclopropyl-3-(trifluoromethyl)-1H-pyrazol-1-yl]pyridin-3-yl}-3-fluoroisonicotinamide;
and pharmaceutically acceptable salts thereof.

14. The method according to any one of claims 1-13, the CRAC modulator is selected from
N-{6-[5-cyclopropyl-3-(trifluoromethyl)-1H-pyrazol-1-yl]pyridin-3-yl}-2-methyl benzamide;
N-{6-[5-cyclopropyl-3-(trifluoromethyl)-1H-pyrazol-1-yl]pyridin-3-yl}-3-fluoroisonicotinamide;
and pharmaceutically acceptable salts thereof.

15. The method according to any one of claims 1-14, wherein the corticosteroid is selected from the group consisting of dexamethasone, betamethasone, prednisolone, methyl prednisolone, prednisone, hydrocortisone, fluticasone, triamcinolone, budesonide or cortisone prednisolone, methylprednisolone, naflocort, deflazacort, halopredone acetate, budesonide, beclomethasone dipropionate, hydrocortisone, triamcinolone acetonide, fluocinolone acetonide, fluocinonide, clocortolone pivalate, methylprednisolone aceponate, dexamethasone palmitoate, tipredane, hydrocortisone aceponate, prednicarbate, alclometasone dipropionate, halometasone, methylprednisolone suleptanate, mometasone, mometasone furoate, mometasone furoate monohydrate, nmexolone, prednisolone farnesylate, ciclesonide, deprodone propionate, fluticasone propionate, halobetasol propionate, loteprednol etabonate, betamethasone butyrate propionate, flunisolide, prednisone, dexamethasone sodium phosphate, triamcinolone, betamethasone 17-valerate, betamethasone, betamethasone dipropionate, hydrocortisone acetate, hydrocortisone sodium succinate, prednisolone sodium phosphate, hydrocortisone probutate, and pharmaceutically acceptable salts thereof.

16. The method according to claim 15, wherein the corticosteroid is selected from the group consisting of dexamethasone, betamethasone, prednisolone, methyl prednisolone, prednisone, hydrocortisone, fluticasone, mometasone, mometasone furoate, mometasone furoate monohydrate, triamcinolone, budesonide, cortisone, and pharmaceutically acceptable salts thereof.

17. The method according to any one of claims 15-16, wherein the corticosteroid is selected from dexamethasone, fluticasone, and pharmaceutically acceptable salts thereof.

18. The method according to any one of claims 1-17, wherein the CRAC modulator is N-{6-[5-cyclopropyl-3-(trifluoromethyl)-1H-pyrazol-1-yl]pyridin-3-yl}-2-methyl benzamide and the corticosteroid is dexamethasone.

19. The method according to any one of claims 1-17, wherein the CRAC modulator is N-{6-[5-cyclopropyl-3-(trifluoromethyl)-1H-pyrazol-1-yl]pyridin-3-yl}-2-methyl benzamide and the corticosteroid is fluticasone.

20. The method according to any one of claims 1-17, wherein the CRAC modulator is N-{6-[5-cyclopropyl-3-(trifluoromethyl)-1H-pyrazol-1-yl]pyridin-3-yl}-2-methyl benzamide and the corticosteroid is mometasone, mometasone furoate or mometasone furoate monohydrate.

21. The method according to any one of claims 1-20, wherein the therapeutically effective amount of (i) the CRAC modulator, and the therapeutically effective amount of (ii) a corticosteroid are administered simultaneously as a combined formulation.

22. The method according to any one of claims 1-20, wherein the therapeutically effective amount of (i) the CRAC modulator, and the therapeutically effective amount of (ii) a corticosteroid are administered sequentially.

23. The method according to claim 22, wherein the therapeutically effective amount of the corticosteroid is administered before the therapeutically effective amount of the CRAC modulator.

24. The method according to any one of claims 1-23, wherein the therapeutically effective amount of the CRAC modulator is administered twice daily to once every three weeks, and the therapeutically effective amount of the corticosteroid is administered twice daily to once every three weeks.

25. The method according to any one of claims 1-24, wherein the autoimmune, respiratory and/or inflammatory disease or condition is selected from the group consisting of asthma, chronic obstructive pulmonary disease, rheumatoid arthritis, inflammatory bowel disease, glomerulonephritis, neuro inflammatory diseases, multiple sclerosis, uveitis, psoriasis, arthritis, vasculitis, dermatitis, osteoarthritis, inflammatory muscle disease, allergic rhinitis, vaginitis, interstitial cystitis, scleroderma, osteoporosis, eczema, allogeneic or xenogeneic transplantation (organ, bone marrow, stem cells and other cells and tissues) graft rejection, graft-versus-host disease, lupus erythematosus, inflammatory disease, type I diabetes, pulmonary fibrosis, dermatomyositis, Sjogren's syndrome, thyroiditis, myasthenia gravis, autoimmune hemolytic anemia, cystic fibrosis, idiopathic pulmonary fibrosis (IPF), chronic relapsing hepatitis, primary biliary cirrhosis, allergic conjunctivitis, atopic dermatitis, and combinations thereof.

26. The method according to any one of claims 1-25, wherein the autoimmune, respiratory and/or inflammatory disease or condition is selected from the group consisting of asthma, rheumatoid arthritis, psoriasis, and chronic obstructive pulmonary disease.

27. The method according to any one of claims 1-26, wherein the CRAC modulator and the corticosteroid are each administered in an amount ranging from about
(i) 0.01 mg to about 1000 mg;
(ii) 0.01 mg to about 500 mg;
(iii) 0.01 mg to about 250 mg; or
(iv) 0.01 mg to about 100 mg;

28. The method according to any one of claims 1-27, wherein
(a) the CRAC modulator is administered in an amount ranging from about
(i) 0.01 mg to about 1000 mg;
(ii) 10 mg to about 500 mg;
(iii) 50 mg to about 250 mg; or
(iv) 50 mg to about 100 mg; and
(b) the corticosteroid is administered in an amount ranging from about 0.01 mg to about 100 mg.

29. The method according to any one of claims 1-28, wherein
(a) the CRAC modulator is administered in an amount ranging from about 10 mg to about 500 mg; and
(b) the corticosteroid is administered in an amount ranging from about 0.01 mg to about 100 mg;

30. The method of any one of claims 1-29, wherein the CRAC modulator and the corticosteroid are administered at a ratio of about 1:100 to about 100:1 by weight.

31. A pharmaceutical composition comprising (i) a CRAC modulator, (ii) a corticosteroid, and (iii) optionally, a pharmaceutically acceptable carrier, glidant, diluent, or excipient.

32. The pharmaceutical composition according to claim 31, wherein the CRAC modulator is
(i) a compound of formula (I) 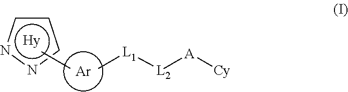
or a tautomer, N-oxide, pharmaceutically acceptable ester or pharmaceutically acceptable salt thereof, wherein
Ring Hy represents 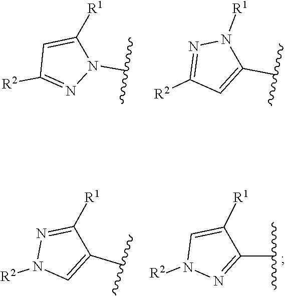
Ring Hy is optionally substituted with R′″;
R¹ and R² are the same or different and are selected from CH₃, CH₂F, CHF₂, CF₃, substituted or unsubstituted C₍₃₋₅₎cycloalkyl, CH₂—OR^{a}, CH₂—NR^{a}R^{b} and COOH;
Ring Ar represents: 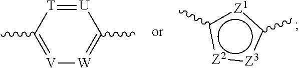
T, U, V and W are the same or different and are independently selected from CR^{a} and N;
Z¹, Z² and Z³ are the same or different and are selected from CR^{a}, CR^{a}R^{b}, O, S and —NR^{a}, with the proviso that at least one of Z¹, Z² and Z³ represents O, S or —NR^{a};
L₁ and L₂ together represent —NH—C(═X)—, —NH—S(═O)_{q}—, —C(═X)NH—, —NH—CR′R″— or —S(═O)_{q}NH—;
A is absent or selected from —(CR′R″)—, O, S(═O)_{q}, C(═X) and —NR^{a};
each occurrence of R′ and R are the same or different and are selected from hydrogen, hydroxy, cyano, halogen, —OR^{a}, —COOR^{a}, —S(═O)_{q}—R^{a}, —NR^{a}R^{b}, —C(═X)—R^{a}, substituted or unsubstituted C₍₁₋₆₎ alkyl group, substituted or unsubstituted C₍₁₋₆₎ alkenyl, substituted or unsubstituted C₍₁₋₆₎ alkynyl, and substituted or unsubstituted C₍₃₋₅₎ cycloalkyl, or R′ and R″ together with the common atom to which they are attached may be joined to form a saturated 3-6 member carbocyclic ring; which may optionally include one or more heteroatoms which may be same or different and are selected from O, NR^{a} and S;
R′″ is selected from hydrogen, hydroxy, cyano, halogen, —OR^{c}, —COOR^{a}, —S(═O)_{q}—R^{a}, —NR^{a}R^{b}, —C(═X)—R^{a}, substituted or unsubstituted C₍₁₋₆₎ alkyl group, substituted or unsubstituted C₍₁₋₆₎ alkenyl, substituted or unsubstituted C₍₁₋₆₎ alkynyl, and substituted or unsubstituted C₍₃₋₅₎cycloalkyl;
each occurrence of X is independently selected from O, S and —NR^{a}
Cy is selected from substituted or unsubstituted cycloalkyl group, substituted or unsubstituted heterocyclyl, substituted or unsubstituted aryl, and substituted or unsubstituted heteroaryl;
each occurrence of R^{a} and R^{b} are the same or different and are selected from hydrogen, nitro, hydroxy, cyano, halogen, —OR^{c}, —S(═O)_{q}—R^{c}, —C(═Y)—R^{c}, —CR^{c}R^{d}—C(═Y)—R^{c}, —CR^{c}R^{d}—Y—CR^{c}R^{d}—, —C(═Y)—NR^{c}R^{d}—, —NRR^{d}—C(═Y)—NR^{c}R^{d}—, —S(═O)_{q}—NR^{c}R^{d}—, —NR^{c}R^{d}—S(═O)_{q}—NR^{c}R^{d}—, —NR^{c}R^{d}—NR^{c}R^{d}—, substituted or unsubstituted alkyl, substituted or unsubstituted alkenyl, substituted or unsubstituted alkynyl, optionally substituted or unsubstituted cycloalkyl, substituted or unsubstituted cycloalkylakyl, substituted or unsubstituted cycloalkenyl, substituted or unsubstituted heterocylyl, substituted or unsubstituted heterocyclylalkyl, substituted or unsubstituted aryl, substituted or unsubstituted arylalkyl, substituted or unsubstituted heteroaryl, and substituted or unsubstituted heteroarylalkyl, or when R^{a} and R^{b} are directly bound to the same atom, they may be joined to form a substituted or unsubstituted saturated or unsaturated 3-10 member ring, which may optionally include one or more heteroatoms which may be the same or different and are selected from O, NR^{c} and S;
each occurrence of R^{c} and R^{d} may be same or different and are selected from hydrogen, nitro, hydroxy, cyano, halogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted heterocyclic group, substituted or unsubstituted heterocyclylalkyl, or when two R^{c} and/or R^{d} substituents are directly bound to the same atom, they may be joined to form a substituted or unsubstituted saturated or unsaturated 3-10 member ring, which may optionally include one or more heteroatoms which are the same or different and are selected from O, NH and S;
each occurrence of Y is independently selected from O, S and —NR^{a}; and
each occurrence of q independently represents 0, 1 or 2; or
(ii)
CM2489;
CM4620;
N-(5-(6-chloro-2,2-difluorobenzo[d][1,3]dioxol-5-yl)pyrazin-2-yl)-2-fluoro-6-methylbenzamide;
N-[4-[3,5-Bis(trifluoromethyl)-1H-pyrazol-1-yl]phenyl]-4-methyl-1,2,3-thiadiazole-5-carboxamide (YM-58483);
2,6-Difluoro-N-{5-[4-methyl-1-(5-methyl-thiazol-2-yl)-1,2,5,6-tetrahydro-pyridin-3-yl]-pyrazin-2-yl}-benzamid (R02959);
2,6-Difluoro-N-(1-(4-hydroxy-2-(trifluoromethyl)benzyl)-1H-pyrazol-3-yl)benzamide (GSK-7975A);
2,6-Difluoro-N-(1-(2-phenoxybenzyl)-1H-pyrazol-3-yl)benzamide (GSK5503A);
N-(2′,5′-Dimethoxy[1,1′-biphenyl]-4-yl)-3-fluoro-4-pyridinecarboxamide (Synta 66);
or a pharmaceutically acceptable salt thereof.

33. The pharmaceutical composition according to any one of claims 31-32, wherein the CRAC modulator is a compound of formula (IA) 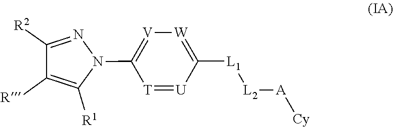
or a tautomer, N-oxide, pharmaceutically acceptable ester, or pharmaceutically acceptable salt thereof, wherein
both R¹ and R² are cyclopropyl or one of R¹ and R² is CF₃ and the other is cyclopropyl;
T is CF or N and U, V, W are independently CH, CF or N;
L₁ and L₂ together represent —NH—C(═X)—, —NH—S(═O)_{q}—, —C(═X)NH—, or —S(═O)_{q}NH— or —NH—CR′R″—;
A is absent or selected from —(CR′R″)— and —NR^{a};
each occurrence of R′ and R″ are the same or different and are independently selected from hydrogen or substituted or unsubstituted C₍₁₋₆₎ alkyl group or R′ and R″ may be joined to form a substituted or unsubstituted saturated or unsaturated 3-6 membered ring, which may optionally include one or more heteroatoms which may be same or different and are selected from O, NR^{a} and S;
R′″ is selected from hydrogen or halogen;
each occurrence of X is independently selected from O, S and —NR^{a};
Cy is selected from 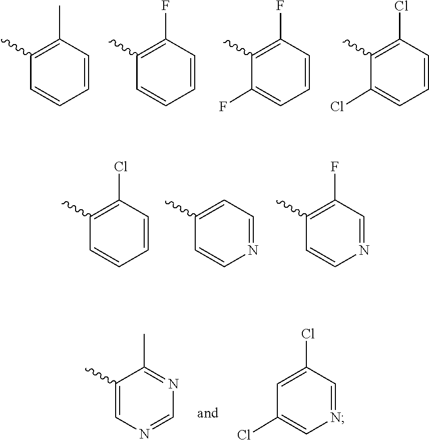
each occurrence of R^{a} is independently selected from hydrogen, nitro, hydroxy, cyano, halogen, —OR^{c}, —S(═O)_{q}—R^{c}, —NR^{c}R^{d}, —C(═Y)—R^{c}, —CR^{c}R^{d}—C(═Y)—R^{c}, —CR^{c}R^{d}—Y—CR^{c}R^{d}—, —C(═Y)—NR^{c}R^{d}—, —NRR^{d}—C(═Y)—NR^{c}R^{d}—, —S(═O)_{q}—NR^{c}R^{d}—, —NR^{c}R^{d}—S(═O)_{q}—NR^{c}R^{d}—, —NR^{c}R^{d}—NR^{c}R^{d}—, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted heterocylyl, substituted or unsubstituted heterocyclylalkyl, substituted or unsubstituted aryl, substituted or unsubstituted arylalkyl, substituted or unsubstituted heteroaryl, and substituted or unsubstituted heteroarylalkyl;
each occurrence of R^{c} and R^{d} may be same or different and are independently selected from hydrogen, nitro, hydroxy, cyano, halogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted heterocyclic group, substituted or unsubstituted heterocyclylalkyl, or when two R^{c} and/or R^{d} substitutents are directly bound to the same atom, they may be joined to form a substituted or unsubstituted saturated or unsaturated 3-10 member ring, which may optionally include one or more heteroatoms which are the same or different and are selected from O, NH and S;
each occurrence of Y is independently selected from O, S and —NR^{a}; and
each occurrence of q independently represents 0, 1 or 2.

34. The pharmaceutical composition according to any one of claims 31-33, wherein the CRAC modulator is a compound of formula (IB) 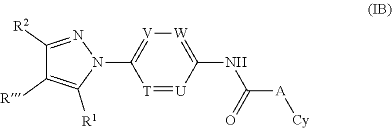
or a tautomer, N-oxide, pharmaceutically acceptable ester or pharmaceutically acceptable salt thereof, wherein
R¹ and R² are both cyclopropyl or one of R¹ and R² is CF₃ and the other is cyclopropyl;
R′″ is selected from hydrogen, hydroxy, cyano, halogen, —OR^{a}, —COOR^{a}, —S(═O)_{q}—R^{a}, —NR^{a}R^{b}, —C(═X)—R^{a}, substituted or unsubstituted C₍₁₋₆₎ alkyl group, substituted or unsubstituted C₍₁₋₆₎ alkenyl, substituted or unsubstituted C₍₁₋₆₎ alkynyl, and substituted or unsubstituted C₍₃₋₅₎cycloalkyl;
T, U, V and W are the same or different and are independently selected from CR^{a} and N;
A is absent or is selected from —CH₂—, —CHMe-, 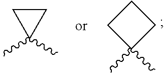
Cy is a bicyclic ring selected from substituted or unsubstituted cycloalkyl group, substituted or unsubstituted heterocyclyl, substituted or unsubstituted aryl, and substituted or unsubstituted heteroaryl;
each occurrence of R^{a} and R^{b} are the same or different and are independently selected from hydrogen, nitro, hydroxy, cyano, halogen, —OR^{c}, —S(═O)_{q}—R^{c}, —NR^{c}R^{d}, —C(═Y)—R^{c}, —CR^{c}R^{d}—C(═Y)—R^{c}, —CR^{c}R^{d}—Y—CR^{c}R^{d}—, —C(═Y)—NR^{c}R^{d}—, —NRR^{d}—C(═Y)—NR^{c}R^{d}—, —S(═O)_{q}—NR^{c}R^{d}—, —NR^{c}R^{d}—S(═O)_{q}—NR^{c}R^{d}—, —NR^{c}R^{d}—NR^{c}R^{d}—, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted heterocylyl, substituted or unsubstituted heterocyclylalkyl, substituted or unsubstituted aryl, substituted or unsubstituted arylalkyl, substituted or unsubstituted heteroaryl, and substituted or unsubstituted heteroarylalkyl, or when R^{a} and R^{b} are directly bound to the same atom, they may be joined to form a substituted or unsubstituted saturated or unsaturated 3-10 member ring, which may optionally include one or more heteroatoms which may be the same or different and are selected from O, NR^{c} and S;
each occurrence of R^{c} and R^{d} may be same or different and are independently selected from hydrogen, nitro, hydroxy, cyano, halogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted heterocyclic group, substituted or unsubstituted heterocyclylalkyl, or when two R^{c} and/or R^{d} substitutents are directly bound to the same atom, they may be joined to form a substituted or unsubstituted saturated or unsaturated 3-10 member ring, which may optionally include one or more heteroatoms which are the same or different and are selected from O, NH and S;
each occurrence of X is independently selected from O, S and —NR^{a};
each occurrence of Y is independently selected from O, S and —NR^{a}; and
each occurrence of q independently represents 0, 1 or 2.

35. The pharmaceutical composition according to any one of claims 31-34, wherein the CRAC modulator is selected from
N-[4-(3,5-dicyclopropyl-1H-pyrazol-1-yl)phenyl]-4-methyl-1,2,3-thiadiazole-5-carboxamide
N-[4-(3,5-dicyclopropyl-1H-pyrazol-1-yl)phenyl]-4-methylthiazole-5-carboxamide
N-[4-(3,5-dicyclopropyl-1H-pyrazol-1-yl)phenyl]-2,4-dimethylthiazole-5-carboxamide
N-[4-(3,5-dicyclopropyl-1H-pyrazol-1-yl)phenyl]-5-methylisoxazole-4-carboxamide
N-[4-(3,5-dicyclopropyl-1H-pyrazol-1-yl)phenyl]-3,5-dimethylisoxazole-4-carboxamide
N-[4-(3,5-dicyclopropyl-1H-pyrazol-1-yl)phenyl]benzamide
N-[4-(3,5-dicyclopropyl-1H-pyrazol-1-yl)phenyl]-2-methylbenzamide
N-[4-(3,5-dicyclopropyl-1H-pyrazol-1-yl)phenyl]-2,6-difluorobenzamide
N-[4-(3,5-dicyclopropyl-1H-pyrazol-1-yl)phenyl]-2,3-difluorobenzamide
N-[4-(3,5-dicyclopropyl-1H-pyrazol-1-yl)phenyl-3-(methylsulfonyl)benzamide
N-[4-(3,5-dicyclopropyl-1H-pyrazol-1-yl)phenyl]-4-(methylsulfonyl)benzamide
2-chloro-N-[4-(3,5-dicyclopropyl-1H-pyrazol-1-yl)phenyl]-5-(methylthio)benzamide
2-chloro-N-[4-(3,5-dicyclopropyl-1H-pyrazol-1-yl)phenyl)-5-(methylsulfonyl)benzamide
N-[4-(3,5-dicyclopropyl-1H-pyrazol-1-yl)phenyl]nicotinamide hydrochloride
N-[4-(3,5-dicyclopropyl-1H-pyrazol-1-yl)phenyl]isonicotinamide hydrochloride
N-[4-(3,5-dicyclopropyl-1H-pyrazol-1-yl)phenyl]-3-fluoroisonicotinamide
3,5-dichloro-N-(4-(3,5-dicyclopropyl-1H-pyrazol-1-yl)phenyl)isonicotinamide
N-[4-(3,5-dicyclopropyl-1H-pyrazol-1-yl)phenyl]-4-methylpyrimidine-5-carboxamide
N-[4-(3,5-dicyclopropyl-1H-pyrazol-1-yl)phenyl]-2-phenylacetamide
N-[4-(3,5-dicyclopropyl-1H-pyrazol-1-yl)phenyl]-2-(4-fluorophenyl)acetamide
N-[4-(3,5-dicyclopropyl-1H-pyrazol-1-yl)phenyl]-1-phenylcyclopropanecarboxamide
N-[4-(3,5-dicyclopropyl-1H-pyrazol-1-yl)phenyl]-2-(pyridin-2-yl)acetamide
N-[4-(3,5-dicyclopropyl-1H-pyrazol-1-yl)phenyl]-2-(pyridin-3-yl)acetamide
N-[4-(3,5-dicyclopropyl-1H-pyrazol-1-yl)phenyl]-2-(pyridin-4-yl)acetamide
N-[4-(3,5-dicyclopropyl-1H-pyrazol-1-yl)phenyl]-2-(piperazin-1-yl)acetamide
N-[4-(3,5-dicyclopropyl-1H-pyrazol-1-yl)phenyl]-2-morpholinoacetamide
N-[4-(3,5-dicyclopropyl-1H-pyrazol-1-yl)phenyl]benzenesulfonamide
N-[4-(3,5-dicyclopropyl-1H-pyrazol-1-yl)-3-fluorophenyl]-4-methyl-1,2,3-thiadiazole-5-carboxamide
N-[4-(3,5-dicyclopropyl-1H-pyrazol-1-yl)-3-fluorophenyl]-4-methylthiazole-5-carboxamide
N-[4-(3,5-dicyclopropyl-1H-pyrazol-1-yl)-3-fluorophenyl]-3,5-dimethylisoxazole-4-carboxamide
N-[4-(3,5-dicyclopropyl-1H-pyrazol-1-yl)-3-fluorophenyl]-2methyl benzamide
N-[4-(3,5-dicyclopropyl-1H-pyrazol-1-yl)-3-fluorophenyl]-2,3-difluorobenzamide
N-[4-(3,5-dicyclopropyl-1H-pyrazol-1-yl)-3-fluorophenyl]-2,6-difluorobenzamide
N-[4-(3,5-dicyclopropyl-1H-pyrazol-1-yl)-3-fluorophenyl]nicotinamide
N-[4-(3,5-dicyclopropyl-1H-pyrazol-1-yl)-3-fluorophenyl]isonicotinamide
N-[4-(3,5-dicyclopropyl-1H-pyrazol-1-yl)-3-fluorophenyl]-4-methylpyrimidine-5-carboxamide
N-[4-(4-chloro-3,5-dicyclopropyl-1H-pyrazol-1-yl)-3-fluorophenyl]-4-methyl-1,2,3-thiadiazole-5-carboxamide
N-[6-(3,5-dicyclopropyl-1H-pyrazol-1-yl)pyridin-3-yl]-4-methyl-1,2,3-thiadiazole-5-carboxamide
N-[6-(3,5-dicyclopropyl-1H-pyrazol-1-yl)pyridin-3-yl]-4-methylthiazole-5-carboxamide
N-[6-(3,5-dicyclopropyl-1H-pyrazol-1-yl)pyridin-3-yl]-2,4-dimethylthiazole-5-carboxamide
N-[6-(3,5-dicyclopropyl-1H-pyrazol-1-yl)pyridin-3-yl]-3,5-dimethylisoxazole-4-carboxamide
6-(3,5-dicyclopropyl-1H-pyrazol-1-yl)-N-o-tolylnicotinamide
N-[6-(3,5-dicyclopropyl-1H-pyrazol-1-yl)pyridin-3-yl]-2-fluorobenzamide
N-[6-(3,5-dicyclopropyl-1H-pyrazol-1-yl)pyridin-3-yl]-2,3-difluorobenzamide
N-[6-(3,5-dicyclopropyl-1H-pyrazol-1-yl)pyridin-3-yl]-2,6-difluorobenzamide
N-[6-(3,5-dicyclopropyl-1H-pyrazol-1-yl)pyridin-3-yl]nicotinamide dihydrochloride
N-[6-(3,5-dicyclopropyl-1H-pyrazol-1-yl)pyridin-3-yl]isonicotinamide
N-[6-(3,5-dicyclopropyl-1H-pyrazol-1-yl)pyridin-3-yl]-3-fluoroisonicotinamide
3,5-dichloro-N-{4-[5-cyclopropyl-3-(trifluoromethyl)-1H-pyrazol-1-yl]phenyl}isonicotinamide
3,5-dichloro-N-[6-(3,5-dicyclopropyl-1H-pyrazol-1-yl)pyridin-3-yl]isonicotinamide
N-[6-(3,5-dicyclopropyl-1H-pyrazol-1-yl)pyridin-3-yl]-4-methylpyrimidine-5-carboxamide
N-{4-[5-cyclopropyl-3-(trifluoromethyl)-1H-pyrazol-1-yl]phenyl}-4-methyl-1,2,3-thiadiazole-5-carboxamide
N-{4-[5-cyclopropyl-3-(trifluoromethyl)-1H-pyrazol-1-yl]phenyl}-4-methylthiazole-5-carboxamide
N-{4-[5-cyclopropyl-3-(trifluoromethyl)-1H-pyrazol-1-yl]phenyl}-N,4-dimethylthiazole-5-carboxamide
N-{4-[5-cyclopropyl-3-(trifluoromethyl)-1H-pyrazol-1-yl]phenyl}-2,4-dimethylthiazole-5-carboxamide
N-{4-[5-cyclopropyl-3-(trifluoromethyl)-1H-pyrazol-1-yl]phenyl}-5-methylisoxazole-4-carboxamide
N-{4-[5-cyclopropyl-3-(trifluoromethyl)-1H-pyrazol-1-yl]phenyl}-3,5-dimethylisoxazole-4-carboxamide
N-{4-[5-cyclopropyl-3-(trifluoromethyl)-1H-pyrazol-1-yl]phenyl}-1-methyl-1H-imidazole-2-carboxamide
N-{4-[3-cyclopropyl-5-(trifluoromethyl)-1H-pyrazol-1-yl]phenyl}-4-methyl-1H-imidazole-5-carboxamide
N-{4-[5-cyclopropyl-3-(trifluoromethyl)-1H-pyrazol-1-yl]phenyl}-2-methylbenzamide
N-{4-[5-cyclopropyl-3-(trifluoromethyl)-1H-pyrazol-1-yl]phenyl}-2,3-difluorobenzamide
N-{4-[5-cyclopropyl-3-(trifluoromethyl)-1H-pyrazol-1-yl]phenyl}-2,6-difluorobenzamide
N-{4-[5-cyclopropyl-3-(trifluoromethyl)-1H-pyrazol-1-yl]phenyl}-3-(methylsulfonyl) benzamide
2-chloro-N-{4-[5-cyclopropyl-3-(trifluoromethyl)-1H-pyrazol-1-yl]phenyl}-5-(methylthio) benzamide
2-chloro-N-{4-[5-cyclopropyl-3-(trifluoromethyl)-1H-pyrazol-1-yl]phenyl}-5-(methylsulfonyl)benzamide
N-{4-[5-cyclopropyl-3-(trifluoromethyl)-1H-pyrazol-1-yl]phenyl}pyridine-4-carboxamide hydrochloride
N-{4-[5-cyclopropyl-3-(trifluoromethyl)-1H-pyrazol-1-yl]phenyl}-3-fluoro isonicotinamide
N-{4-[5-cyclopropyl-3-(trifluoromethyl)-1H-pyrazol-1-yl]phenyl}-4-methylpyrimidine-5-carboxamide
N-{4-[5-cyclopropyl-3-(trifluoromethyl)-1H-pyrazol-1-yl]phenyl}-2,4-dimethyl pyrimidine-5-carboxamide
N-{4-[5-cyclopropyl-3-(trifluoromethyl)-1H-pyrazol-1-yl]phenyl}-2-(4-fluorophenyl)acetamide
N-{4-[5-cyclopropyl-3-(trifluoromethyl)-1H-pyrazol-1-yl]phenyl}-2-(pyridin-2-yl)acetamide
N-{4-[5-cyclopropyl-3-(trifluoromethyl)-1H-pyrazol-1-yl]phenyl}-2-(pyridin-3-yl)acetamide
N-{4-[5-cyclopropyl-3-(trifluoromethyl)-1H-pyrazol-1-yl]phenyl}-2-(pyridin-4-yl)acetamide
4-[5-cyclopropyl-3-(trifluoromethyl)-1H-pyrazol-1-yl]-N-[(4-methylthiazol-5-yl)methyl]aniline
1-{4-[5-cyclopropyl-3-(trifluoromethyl)-1H-pyrazol-1-yl]phenyl}-3-(4-methyl-1,2,3-thiadiazol-5-yl)urea
1-{4-[5-cyclopropyl-3-(trifluoromethyl)-1H-pyrazol-1-yl]phenyl}-3-(4-methylthiazol-5-yl)urea
1-{4-[5-cyclopropyl-3-(trifluoromethyl)-1H-pyrazol-1-yl]phenyl}-3-(4-methylpyrimidin-5-yl)urea
4-[5-cyclopropyl-3-(trifluoromethyl)-1H-pyrazol-1-yl]-N-(4-methylthiazol-5-yl) benzamide
4-[5-cyclopropyl-3-(trifluoromethyl)-1H-pyrazol-1-yl]-N-(2,6-difluorophenyl) benzamide
N-{4-[4-chloro-5-cyclopropyl-3-(trifluoromethyl)-1H-pyrazol-1-yl]phenyl}-4-methylthiazole-5-carboxamide
N-{4-[4-chloro-5-cyclopropyl-3-(trifluoromethyl)-1H-pyrazol-1-yl]phenyl}-2-(pyridin-2-yl)acetamide
N-{4-[5-cyclopropyl-3-(trifluoromethyl)-1H-pyrazol-1-yl]-3-fluorophenyl}-4-methyl-1,2,3-thiadiazole-5-carboxamide
N-{4-[5-cyclopropyl-3-(trifluoromethyl)-1H-pyrazol-1-yl]-3-fluorophenyl}-4-methylthiazole-5-carboxamide
N-{4-[5-cyclopropyl-3-(trifluoromethyl)-1H-pyrazol-1-yl]-3-fluorophenyl}-5-methylisoxazole-4-carboxamide
N-{4-[5-cyclopropyl-3-(trifluoromethyl)-1H-pyrazol-1-yl]-3-fluorophenyl}-3,5-dimethylisoxazole-4-carboxamide
N-{4-[5-cyclopropyl-3-(trifluoromethyl)-1H-pyrazol-1-yl]-3-fluorophenyl}-2-methylbenzamide
N-{4-[5-cyclopropyl-3-(trifluoromethyl)-1H-pyrazol-1-yl]-3-fluorophenyl}-2,3-difluorobenzamide
N-{4-[5-cyclopropyl-3-(trifluoromethyl)-1H-pyrazol-1-yl]-3-fluorophenyl}-2,6-difluorobenzamide
N-{4-[5-cyclopropyl-3-(trifluoromethyl)-1H-pyrazol-1-yl]-3-fluorophenyl}nicotinamide
N-{4-[5-cyclopropyl-3-(trifluoromethyl)-1H-pyrazol-1-yl]-3-fluorophenyl}isonicotinamide
N-{4-[5-cyclopropyl-3-(trifluoromethyl)-1H-pyrazol-1-yl]-3-fluorophenyl}-3-fluoroisonicotinamide
3,5-dichloro-N-{4-[5-cyclopropyl-3-(trifluoromethyl)-1H-pyrazol-1-yl]-3-fluorophenyl}isonicotinamide
N-{4-[5-cyclopropyl-3-(trifluoromethyl)-1H-pyrazol-1-yl]-3-fluorophenyl}-4-methylpyrimidine-5-carboxamide
N-{4-[5-cyclopropyl-3-(trifluoromethyl)-1H-pyrazol-1-yl]-3-fluorophenyl}-N,4-dimethylpyrimidine-5-carboxamide
N-{4-[4-chloro-5-cyclopropyl-3-(trifluoromethyl)-1H-pyrazol-1-yl]-3-fluorophenyl}-4-methyl-1,2,3-thiadiazole-5-carboxamide
N-{4-[4-chloro-5-cyclopropyl-3-(trifluoromethyl)-1H-pyrazol-1-yl]-3-fluorophenyl}-2-(pyridin-2-yl)acetamide
1-{4-[5-cyclopropyl-3-(trifluoromethyl)-1H-pyrazol-1-yl]-3-fluorophenyl}-3-(4-methylpyrimidin-5-yl)urea
N-{4-[5)-cyclopropyl-3-(trifluromethyl)-1H-pyrazol-1-yl]3-fluorophenyl}-2,6-dichloro benzamide
4-[5-cyclopropyl-3-(trifluoromethyl)-1H-pyrazol-1-yl]-N-(2,3-difluorophenyl)-3-fluorobenzamide
4-[5-cyclopropyl-3-(trifluoromethyl)-1H-pyrazol-1-yl]-N-(2,6-difluorophenyl)-3-fluorobenzamide
N-{6-[5-cyclopropyl-3-(trifluoromethyl)-1H-pyrazol-1-yl]pyridin-3-yl}-4-methyl-1,2,3-thiadiazole-5-carboxamide
N-{6-[5-cyclopropyl-3-(trifluoromethyl)-1H-pyrazol-1-yl]pyridin-3-yl}-4-methylthiazole-5-carboxamide
N-{6-[5-cyclopropyl-3-(trifluoromethyl)-1H-pyrazol-1-yl]pyridin-3-yl}-3,5-dimethylisoxazole-4-carboxamide
N-{6-[5-cyclopropyl-3-(trifluoromethyl)-1H-pyrazol-1-yl]pyridin-3-yl}-2-methylbenzamide
2-chloro-N-{6-[5-cyclopropyl-3-(trifluoromethyl)-1H-pyrazol-1-yl]pyridin-3-yl}benzamide
N-(6-(5-cyclopropyl-3-(trifluoromethyl)-1H-pyrazol-1-yl)pyridin-3-yl)-2-fluorobenzamide
N-{6-[5-cyclopropyl-3-(trifluoromethyl)-1H-pyrazol-1-yl]pyridin-3-yl}-2,3-difluorobenzamide
N-{6-[5-cyclopropyl-3-(trifluoromethyl)-1H-pyrazol-1-yl]pyridin-3-yl}-2,6-difluorobenzamide
N-{6-[5-cyclopropyl-3-(trifluoromethyl)-1H-pyrazol-1-yl]pyridin-3-yl}picolinamide
N-{6-[5-cyclopropyl-3-(trifluoromethyl)-1H-pyrazol-1-yl]pyridin-3-yl}-3-methylpicolinamide
N-{6-[5-cyclopropyl-3-(trifluoromethyl)-1H-pyrazol-1-yl]pyridin-3-yl}nicotinamide
N-{6-[5-cyclopropyl-3-(trifluoromethyl)-1H-pyrazol-1-yl]pyridin-3-yl}-2-methylnicotinamide
N-{6-[5-cyclopropyl-3-(trifluoromethyl)-1H-pyrazol-1-yl]pyridin-3-yl}isonicotinamide
N-{6-[5-cyclopropyl-3-(trifluoromethyl)-1H-pyrazol-1-yl]pyridin-3-yl}-3-fluoroisonicotinamide
3,5-dichloro-N-{6-[5-cyclopropyl-3-(trifluoromethyl)-1H-pyrazol-1-yl]pyridin-3-yl}isonicotinamide
N-{6-[5-cyclopropyl-3-(trifluoromethyl)-1H-pyrazol-1-yl]pyridin-3-yl}-4-methylpyrimidine-5-carboxamide
N-{6-[5-cyclopropyl-3-(trifluoromethyl)-1H-pyrazol-1-yl]pyridin-3-yl}-2-(pyridin-2-yl)acetamide
N-{6-[5-cyclopropyl-3-(trifluoromethyl)-1H-pyrazol-1-yl]pyridin-3-yl}-2-(pyridin-4-yl)acetamide
N-{4-[4-chloro-5-cyclopropyl-3-(trifluoromethyl)-1H-pyrazol-1-yl]-3-fluorophenyl}-4-methylpyrimidine-5-carboxamide
1-{6-[3-cyclopropyl-5-(trifluoromethyl)-1H-pyrazol-1-yl]pyridin-3-yl}-3-(4-methylthiazol-5-yl)urea
6-[5-cyclopropyl-3-(trifluoromethyl)-1H-pyrazol-1-yl]-N-(2,3-difluorophenyl) nicotinamide
6-[5-cyclopropyl-3-(trifluoromethyl)-1H-pyrazol-1-yl]-N-(2,6-difluorophenyl) nicotinamide
N-{6-[4-chloro-5-cyclopropyl-3-(trifluoromethyl)-1H-pyrazol-1-yl]pyridin-3-yl}-4-methylthiazole-5-carboxamide
N-{2-[5-cyclopropyl-3-(trifluoromethyl)-1H-pyrazol-1-yl]pyrimidin-5-yl}-2,6-difluorobenzamide
N-{4-[5-(fluoromethyl)-3-(trifluoromethyl)-1H-pyrazol-1-yl]phenyl}-4-methylthiazole-5-carboxamide
N-{4-[5-(difluoromethyl)-3-(trifluoromethyl)-1H-pyrazol-1-yl]phenyl}-4-methylthiazole-5-carboxamide
3,5-dichloro-N-[4-(3,5-dicyclopropyl-1H-pyrazol-1-yl)-3-fluorophenyl]isonicotinamide
N-(2-chloro-6-fluorophenyl)-4-[5-cyclopropyl-3-(trifluoromethyl)-1H-pyrazol-1-yl]-3-fluorobenzamide
N-{2-[5-cyclopropyl-3-(trifluoromethyl)-1H-pyrazol-1-yl]pyrimidin-5-yl}-4-methylthiazole-5-carboxamide
N-{4-[5-cyclopropyl-3-(trifluoromethyl)-1H-pyrazol-1-yl]-3,5-difluorophenyl}-4-methylpyrimidine-5-carboxamide
{4-[5-cyclopropyl-3-(trifluoromethyl)-1H-pyrazol-1-yl]-3-fluorophenyl}-1-phenylcyclobutanecarboxamide
N-{4-[5-cyclopropyl-3-(trifluoromethyl)-1H-pyrazol-1-yl]-3-fluorophenyl}-4-methyloxazole-5-carboxamide
N-{2-[5-cyclopropyl-3-(trifluoromethyl)-1H-pyrazol-1-yl]pyrimidin-5-yl}-4-methylpyrimidine-5-carboxamide
4-[5-cyclopropyl-3-(trifluoromethyl)-1H-pyrazol-1-yl]-3-fluoro-N-(4-methylpyrimidin-5-yl) benzamide and
N-{4-[3-cyclopropyl-5-(difluoromethyl)-1H-pyrazol-1-yl]-3-fluorophenyl}-2,6-difluorobenzamide;
N-{4-[5-cyclopropyl-3-(difluoromethyl)-1H-pyrazol-1-yl]-3-fluorophenyl}-2,6-difluorobenzamide
N-[4-(3,5-dicyclopropyl-1H-pyrazol-1-yl)phenyl]-1H-benzo[d]imidazole-6-carboxamide
N-[4-(3,5-dicyclopropyl-1H-pyrazol-1-yl)phenyl]-1H-benzo[d][1,2,3]triazole-6-carboxamide
N-[4-(3,5-dicyclopropyl-1H-pyrazol-1-yl)phenyl]quinoline-6-carboxamide hydrochloride
N-[4-(3,5-dicyclopropyl-1H-pyrazol-1-yl)phenyl]quinoxaline-6-carboxamide
2-(1H-benzo[d]imidazol-1-yl)-N-[4-(3,5-dicyclopropyl-1H-pyrazol-1-yl)phenyl]acetamide
2-(1H-benzo[d][1,2,3]triazol-1-yl)-N-[4-(3,5-dicyclopropyl-1H-pyrazol-1-yl)phenyl]acetamide
N-[4-(3,5-dicyclopropyl-1H-pyrazol-1-yl)phenyl]-2-(1H-indol-3-yl)acetamide
N-[4-(3,5-dicyclopropyl-1H-pyrazol-1-yl)phenyl]-2-(imidazo[1,2-a]pyridin-2-yl)acetamide hydrochloride
N-[4-(3,5-dicyclopropyl-1H-pyrazol-1-yl)phenyl]-2-(quinolin-6-yl)acetamide:
N-[4-(3,5-dicyclopropyl-1H-pyrazol-1-yl)phenyl]-2-(quinolin-6-yl)acetamide hydrochloride
2-(1H-benzo[d][1,2,3]triazol-1-yl)-N-(4-(3,5-dicyclopropyl-1H-pyrazol-1-yl)-3-fluorophenyl)acetamide
N-[4-(3,5-dicyclopropyl-1H-pyrazol-1-yl)-3-fluorophenyl]-2-(quinolin-6-yl)acetamide hydrochloride
N-[6-(3,5-dicyclopropyl-1H-pyrazol-1-yl)pyridin-3-yl]quinoline-6-carboxamide dihydrochloride
N-[6-(3,5-dicyclopropyl-1H-pyrazol-1-yl)pyridin-3-yl]quinoxaline-6-carboxamide
2-(1H-benzo[d][1,2,3]triazol-1-yl)-N-[6-(3,5-dicyclopropyl-1H-pyrazol-1-yl)pyridin-3-yl]acetamide
N-[6-(3,5-dicyclopropyl-1H-pyrazol-1-yl)pyridin-3-yl]-2-(quinolin-6-yl)acetamidedihydrochloride
N-{4-[5-cyclopropyl-3-(trifluoromethyl)-1H-pyrazol-1-yl]phenyl}quinoline-6-carboxamide hydrochloride
N-{4-[5-cyclopropyl-3-(trifluoromethyl)-1H-pyrazol-1-yl]phenyl}quinoxaline-6-carboxamide
2-(1H-benzo[d]imidazol-1-yl)-N-{4-[5-cyclopropyl-3-(trifluoromethyl)-1H-pyrazol-1-yl]phenyl}acetamide
2-(1H-benzo[d][1,2,3]triazol-1-yl)-N-{4-[5-cyclopropyl-3-(trifluoromethyl)-1H-pyrazol-1-yl]phenyl}acetamide
2-(2H-benzo[d][1,2,3]triazol-2-yl)-N-{4-[5-cyclopropyl-3-(trifluoromethyl)-1H-pyrazol-1-yl]phenyl}acetamide
2-(3H-[1,2,3]triazolo[4,5-b]pyridin-3-yl)-N-{4-[5-cyclopropyl-3-(trifluoromethyl)-1H-pyrazol-1-yl]phenyl}acetamide
(S)-2-(3H-[1,2,3]triazolo[4,5-b]pyridin-3-yl)-N-{4-[5-cyclopropyl-3-(trifluoromethyl)-1H-pyrazol-1-yl]phenyl}propanamide
2-(6-amino-9H-purin-9-yl)-N-{4-[5-cyclopropyl-3-(trifluoromethyl)-1H-pyrazol-1-yl]phenyl}acetamide
N-(4-(5-cyclopropyl-3-(trifluoromethyl)-1H-pyrazol-1-yl)phenyl)-2-(1,3-dimethyl-2,6-dioxo-2,3-dihydro-1H-purin-7(6H)-yl)acetamide
N-{4-[5-cyclopropyl-3-(trifluoromethyl)-1H-pyrazol-1-yl)phenyl)-2-(imidazo[1,2-a]pyridin-2-yl)acetamide hydrochloride
N-{4-[5-cyclopropyl-3-(trifluoromethyl)-1H-pyrazol-1-yl]phenyl}-2-(quinolin-6-yl)acetamide hydrochloride
N-{4-[5-cyclopropyl-3-(trifluoromethyl)-1H-pyrazol-1-yl]phenyl}-2-(quinolin-6-yl)propanamide hydrochloride
N-{4-[5-cyclopropyl-3-(trifluoromethyl)-1H-pyrazol-1-yl]-3-fluorophenyl}-1H-benzo[d][1,2,3]triazole-6-carboxamide
2-(1H-benzo[d][1,2,3]triazol-1-yl)-N-{4-[5-cyclopropyl-3-(trifluoromethyl)-1H-pyrazol-1-yl]-3-fluorophenyl}acetamide
N-{6-[5-cyclopropyl-3-(trifluoromethyl)-1H-pyrazol-1-yl]pyridin-3-yl}-1H-benzo[d][1,2,3]triazole-5-carboxamide
2-(1H-benzo[d][1,2,3]triazol-1-yl)-N-{6-[5-cyclopropyl-3-(trifluoromethyl)-1H-pyrazol-1-yl]pyridin-3-yl}acetamide
2-(2H-benzo[d][1,2,3]triazol-2-yl)-N-{6-[5-cyclopropyl-3-(trifluoromethyl)-1H-pyrazol-1-yl]acetamide
N-{6-[5-cyclopropyl-3-(trifluoromethyl)-1H-pyrazol-1-yl]pyridin-3-yl}-2-(quinolin-6-yl)acetamide hydrochloride
2-(1H-benzo[d][1,2,3]triazol-1-yl)-N-{6-[4-chloro-5-cyclopropyl-3-(trifluoromethyl)-1H-pyrazol-1-yl]pyridin-3-yl}acetamide
4-[5-cyclopropyl-3-(trifluoromethyl)-1H-pyrazol-1-yl]-3-fluoro-N-(quinolin-6-ylmethyl) benzamide hydrochloride and
1-[4-(3,5-dicyclopropyl-1H-pyrazol-1-yl)phenyl]-3-(quinolin-6-yl)urea;
and pharmaceutically acceptable salts thereof.

36. The pharmaceutical composition according to any one of claims 31-35, wherein the CRAC modulator is selected from CM2489;
CM4620;
N-(5-(6-chloro-2,2-difluorobenzo[d][1,3]dioxol-5-yl)pyrazin-2-yl)-2-fluoro-6-methylbenzamide;
N-[4-[3,5-Bis(trifluoromethyl)-1H-pyrazol-1-yl]phenyl]-4-methyl-1,2,3-thiadiazole-5-carboxamide (YM-58483);
2,6-Difluoro-N-{5-[4-methyl-1-(5-methyl-thiazol-2-yl)-1,2,5,6-tetrahydro-pyridin-3-yl]-pyrazin-2-yl}-benzamid (R02959);
2,6-Difluoro-N-(1-(4-hydroxy-2-(trifluoromethyl)benzyl)-1H-pyrazol-3-yl)benzamide (GSK-7975A);
2,6-Difluoro-N-(1-(2-phenoxybenzyl)-1H-pyrazol-3-yl)benzamide (GSK5503A);
N-(2′,5′-Dimethoxy[1,1′-biphenyl]-4-yl)-3-fluoro-4-pyridinecarboxamide (Synta 66);
N-{6-[5-cyclopropyl-3-(trifluoromethyl)-1H-pyrazol-1-yl]pyridin-3-yl}-2-methylbenzamide;
N-{6-[5-cyclopropyl-3-(trifluoromethyl)-1H-pyrazol-1-yl]pyridin-3-yl}-3-fluoroisonicotinamide;
and pharmaceutically acceptable salts thereof.

37. The pharmaceutical composition according to any one of claims 31-36, wherein the CRAC modulator is selected from
N-{6-[5-cyclopropyl-3-(trifluoromethyl)-1H-pyrazol-1-yl]pyridin-3-yl}-2-methyl benzamide;
N-{6-[5-cyclopropyl-3-(trifluoromethyl)-1H-pyrazol-1-yl]pyridin-3-yl}-3-fluoroisonicotinamide;
and pharmaceutically acceptable salts thereof.

38. The pharmaceutical composition according to any one of claims 31-37, wherein the corticosteroid is selected from the group consisting of dexamethasone, betamethasone, prednisolone, methyl prednisolone, prednisone, hydrocortisone, fluticasone, triamcinolone, budesonide or cortisone prednisolone, methylprednisolone, naflocort, deflazacort, halopredone acetate, budesonide, beclomethasone dipropionate, hydrocortisone, triamcinolone acetonide, fluocinolone acetonide, fluocinonide, clocortolone pivalate, methylprednisolone aceponate, dexamethasone palmitoate, tipredane, hydrocortisone aceponate, prednicarbate, alclometasone dipropionate, halometasone, methylprednisolone suleptanate, mometasone, mometasone furoate, mometasone furoate monohydrate, rimexolone, prednisolone farnesylate, ciclesonide, deprodone propionate, fluticasone propionate, halobetasol propionate, loteprednol etabonate, betamethasone butyrate propionate, flunisolide, prednisone, dexamethasone sodium phosphate, triamcinolone, betamethasone 17-valerate, betamethasone, betamethasone dipropionate, hydrocortisone acetate, hydrocortisone sodium succinate, prednisolone sodium phosphate, hydrocortisone probutate, and pharmaceutically acceptable salts thereof.

39. The pharmaceutical composition according to claim 38, wherein the corticosteroid is selected from the group consisting of dexamethasone, betamethasone, prednisolone, methyl prednisolone, prednisone, hydrocortisone, fluticasone, mometasone, mometasone furoate, mometasone furoate monohydrate, triamcinolone, budesonide, cortisone, and pharmaceutically acceptable salts thereof.

40. The pharmaceutical composition according to any one of claims 38-39, wherein the corticosteroid is selected from dexamethasone, fluticasone, and pharmaceutically acceptable salts thereof.

41. The pharmaceutical composition according to any one of claims 31-40, wherein the CRAC modulator is N-{6-[5-cyclopropyl-3-(trifluoromethyl)-1H-pyrazol-1-yl]pyridin-3-yl}-2-methyl benzamide and the corticosteroid is dexamethasone.

42. The pharmaceutical composition according to any one of claims 31-40, wherein the CRAC modulator is N-{6-[5-cyclopropyl-3-(trifluoromethyl)-1H-pyrazol-1-yl]pyridin-3-yl}-2-methyl benzamide and the corticosteroid is fluticasone.

43. The pharmaceutical composition according to any one of claims 31-40, wherein the CRAC modulator is N-{6-[5-cyclopropyl-3-(trifluoromethyl)-1H-pyrazol-1-yl]pyridin-3-yl}-2-methyl benzamide and the corticosteroid is mometasone, mometasone furoate or mometasone furoate monohydrate.

44. The pharmaceutical composition of any one of claims 31-42, wherein the composition comprises about
(i) 0.01 mg to about 1000 mg;
(ii) 0.01 mg to about 500 mg;
(iii) 0.01 mg to about 250 mg; or
(iv) 0.01 mg to about 100 mg of each of the CRAC modulator and the corticosteroid.

45. The pharmaceutical composition of any one of claims 31-44, wherein the composition comprises about
(a)
(i) 0.01 mg to about 1000 mg;
(ii) 10 mg to about 500 mg;
(iii) 50 mg to about 250 mg; or
(iv) 50 mg to about 100 mg;
of the CRAC modulator; and
(b) about 0.01 mg to about 100 mg of the corticosteroid

46. The pharmaceutical composition of any one of claims 31-45, wherein the composition comprises about 10 mg to about 500 mg of CRAC modulator; and about 0.01 mg to about 100 mg of the corticosteroid.

47. The pharmaceutical composition according to any one of claims 31-46, for use in a method of treating an autoimmune, respiratory and/or inflammatory disease or condition selected from the group consisting of asthma, chronic obstructive pulmonary disease, rheumatoid arthritis, inflammatory bowel disease, glomerulonephritis, neuroinflammatory diseases, multiple sclerosis, uveitis, psoriasis, arthritis, vasculitis, dermatitis, osteoarthritis, inflammatory muscle disease, allergic rhinitis, vaginitis, interstitial cystitis, scleroderma, osteoporosis, eczema, allogeneic or xenogeneic transplantation (organ, bone marrow, stem cells and other cells and tissues) graft rejection, graft-versus-host disease, lupus erythematosus, inflammatory disease, type I diabetes, pulmonary fibrosis, dermatomyositis, Sjogren's syndrome, thyroiditis (e.g., Hashimoto's and autoimmune thyroiditis), myasthenia gravis, autoimmune hemolytic anemia, cystic fibrosis, Idiopathic pulmonary fibrosis (IPF), chronic relapsing hepatitis, primary biliary cirrhosis, allergic conjunctivitis and atopic dermatitis, and combinations thereof.

48. The use of a pharmaceutical composition according to any one of claims 31-46, in the manufacture of a medicament for the treatment of an autoimmune, respiratory or inflammatory disease or condition selected from asthma, chronic obstructive pulmonary disease, rheumatoid arthritis, inflammatory bowel disease, glomerulonephritis, neuroinflammatory diseases, multiple sclerosis, uveitis, psoriasis, arthritis, vasculitis, dermatitis, osteoarthritis, inflammatory muscle disease, allergic rhinitis, vaginitis, interstitial cystitis, scleroderma, osteoporosis, eczema, allogeneic or xenogeneic transplantation (organ, bone marrow, stem cells and other cells and tissues) graft rejection, graft-versus-host disease, lupus erythematosus, inflammatory disease, type I diabetes, pulmonary fibrosis, dermatomyositis, Sjogren's syndrome, thyroiditis (e.g., Hashimoto's and autoimmune thyroiditis), myasthenia gravis, autoimmune hemolytic anemia, cystic fibrosis, Idiopathic pulmonary fibrosis (IPF), chronic relapsing hepatitis, primary biliary cirrhosis, allergic conjunctivitis and atopic dermatitis, and combinations thereof.

49. A kit for treating an autoimmune, respiratory or inflammatory disease or condition, the kit comprising:
(i) a CRAC modulator, and (ii) a corticosteroid, either in a single pharmaceutical composition or in separate pharmaceutical compositions,
(ii) optionally, instructions for treating the autoimmune, respiratory or inflammatory disease or condition with the CRAC modulator and corticosteroid; and
(iii) optionally, a container for placing the pharmaceutical composition or pharmaceutical compositions.

50. The kit according to claim 49, wherein the CRAC modulator and corticosteroid are for use in the treatment of an autoimmune, respiratory or inflammatory disease or condition selected from asthma, chronic obstructive pulmonary disease, rheumatoid arthritis, inflammatory bowel disease, glomerulonephritis, neuroinflammatory diseases, multiple sclerosis, uveitis, psoriasis, arthritis, vasculitis, dermatitis, osteoarthritis, inflammatory muscle disease, allergic rhinitis, vaginitis, interstitial cystitis, scleroderma, osteoporosis, eczema, allogeneic or xenogeneic transplantation (organ, bone marrow, stem cells and other cells and tissues) graft rejection, graft-versus-host disease, lupus erythematosus, inflammatory disease, type I diabetes, pulmonary fibrosis, dermatomyositis, Sjogren's syndrome, thyroiditis (e.g., Hashimoto's and autoimmune thyroiditis), myasthenia gravis, autoimmune hemolytic anemia, cystic fibrosis, Idiopathic pulmonary fibrosis (IPF), chronic relapsing hepatitis, primary biliary cirrhosis, allergic conjunctivitis and atopic dermatitis.

51. The kit according to any one of claim 49 or 50, wherein the corticosteroid is selected from the group consisting of dexamethasone, betamethasone, prednisolone, methyl prednisolone, prednisone, hydrocortisone, fluticasone, triamcinolone, budesonide or cortisone prednisolone, methylprednisolone, naflocort, deflazacort, halopredone acetate, budesonide, beclomethasone dipropionate, hydrocortisone, triamcinolone acetonide, fluocinolone acetonide, fluocinonide, clocortolone pivalate, methylprednisolone aceponate, dexamethasone palmitoate, tipredane, hydrocortisone aceponate, prednicarbate, alclometasone dipropionate, halometasone, methylprednisolone suleptanate, mometasone, mometasone furoate, mometasone furoate monohydrate, nmexolone, prednisolone farnesylate, ciclesonide, deprodone propionate, fluticasone propionate, halobetasol propionate, loteprednol etabonate, betamethasone butyrate propionate, flunisolide, prednisone, dexamethasone sodium phosphate, triamcinolone, betamethasone 17-valerate, betamethasone, betamethasone dipropionate, hydrocortisone acetate, hydrocortisone sodium succinate, prednisolone sodium phosphate, hydrocortisone probutate, and pharmaceutically acceptable salts thereof.

52. The kit according to claim 51, wherein the corticosteroid is selected from the group consisting of dexamethasone, betamethasone, prednisolone, methyl prednisolone, prednisone, hydrocortisone, fluticasone, mometasone, mometasone furoate, mometasone furoate monohydrate, triamcinolone, budesonide, cortisone, and pharmaceutically acceptable salts thereof.

53. The kit according to any one of claims 49-52, wherein the CRAC modulator is
(i) a compound of formula (I) 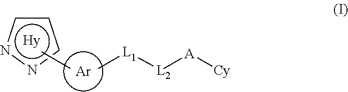
or a tautomer, N-oxide, pharmaceutically acceptable ester or pharmaceutically acceptable salt thereof, wherein
Ring Hy represents 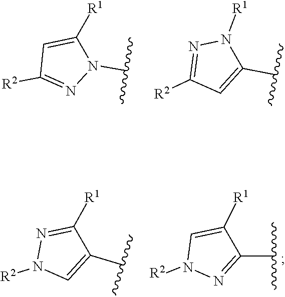
Ring Hy is optionally substituted with R′″;
R¹ and R² are the same or different and are selected from CH₃, CH₂F, CHF₂, CF₃, substituted or unsubstituted C₍₃₋₅₎cycloalkyl, CH₂—OR^{a}, CH₂—NR^{a}R^{b} and COOH;
Ring Ar represents: 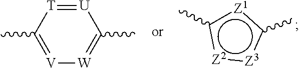
T, U, V and W are the same or different and are independently selected from CR^{a} and N;
Z¹, Z² and Z³ are the same or different and are selected from CR^{a}, CR^{a}R^{b}, O, S and —NR^{a}, with the proviso that at least one of Z¹, Z² and Z³ represents O, S or —NR^{a};
L₁ and L₂ together represent —NH—C(═X)—, —NH—S(═O)_{q}—, —C(═X)NH—, —NH—CR′R″— or —S(═O)_{q}NH—;
A is absent or selected from —(CR′R″)—, O, S(═O)_{q}, C(═X) and —NR^{a};
each occurrence of R′ and R″ are the same or different and are selected from hydrogen, hydroxy, cyano, halogen, —OR^{a}, —COOR^{a}, —S(═O)_{q}—R^{a}, —NR^{a}R^{b}, —C(═X)—R^{a}, substituted or unsubstituted C₍₁₋₆₎ alkyl group, substituted or unsubstituted C₍₁₋₆₎ alkenyl, substituted or unsubstituted C₍₁₋₆₎ alkynyl, and substituted or unsubstituted C₍₃₋₅₎ cycloalkyl, or R′ and R″ together with the common atom to which they are attached may be joined to form a saturated 3-6 member carbocyclic ring; which may optionally include one or more heteroatoms which may be same or different and are selected from O, NR^{a} and S;
R′″ is selected from hydrogen, hydroxy, cyano, halogen, —OR^{a}, —COOR^{a}, —S(═O)_{q}—R^{a}, —NR^{a}R^{b}, —C(═X)—R^{a}, substituted or unsubstituted C₍₁₋₆₎ alkyl group, substituted or unsubstituted C₍₁₋₆₎ alkenyl, substituted or unsubstituted C₍₁₋₆₎ alkynyl, and substituted or unsubstituted C₍₃₋₅₎cycloalkyl;
each occurrence of X is independently selected from O, S and —NR^{a};
Cy is selected from substituted or unsubstituted cycloalkyl group, substituted or unsubstituted heterocyclyl, substituted or unsubstituted aryl, and substituted or unsubstituted heteroaryl;
each occurrence of R^{a} and R^{b} are the same or different and are selected from hydrogen, nitro, hydroxy, cyano, halogen, —OR^{c}, —S(═O)_{q}—R^{c}, —C(═Y)—R^{c}, —CR^{c}R^{d}—C(═Y)—R^{c}, —CR^{c}R^{d}—Y—CR^{c}R^{d}—, —C(═Y)—NR^{c}R^{d}—, —NRR^{d}—C(═Y)—NR^{c}R^{d}—, —S(═O)_{q}—NR^{c}R^{d}—, —NR^{c}R^{d}—S(═O)_{q}—NR^{c}R^{d}—, —NR^{c}R^{d}—NR^{c}R^{d}—, substituted or unsubstituted alkyl, substituted or unsubstituted alkenyl, substituted or unsubstituted alkynyl, optionally substituted or unsubstituted cycloalkyl, substituted or unsubstituted cycloalkylakyl, substituted or unsubstituted cycloalkenyl, substituted or unsubstituted heterocylyl, substituted or unsubstituted heterocyclylalkyl, substituted or unsubstituted aryl, substituted or unsubstituted arylalkyl, substituted or unsubstituted heteroaryl, and substituted or unsubstituted heteroarylalkyl, or when R^{a} and R^{b} are directly bound to the same atom, they may be joined to form a substituted or unsubstituted saturated or unsaturated 3-10 member ring, which may optionally include one or more heteroatoms which may be the same or different and are selected from O, NR^{c} and S;
each occurrence of R^{c} and R^{d} may be same or different and are selected from hydrogen, nitro, hydroxy, cyano, halogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted heterocyclic group, substituted or unsubstituted heterocyclylalkyl, or when two R^{c} and/or R^{d} substituents are directly bound to the same atom, they may be joined to form a substituted or unsubstituted saturated or unsaturated 3-10 member ring, which may optionally include one or more heteroatoms which are the same or different and are selected from O, NH and S;
each occurrence of Y is independently selected from O, S and —NR^{a}; and
each occurrence of q independently represents 0, 1 or 2; or
(ii)
CM2489,
CM4620,
N-(5-(6-chloro-2,2-difluorobenzo[d][1,3]dioxol-5-yl)pyrazin-2-yl)-2-fluoro-6-methylbenzamide;
N-[4-[3,5-Bis(trifluoromethyl)-1H-pyrazol-1-yl]phenyl]-4-methyl-1,2,3-thiadiazole-5-carboxamide (YM-58483),
2,6-Difluoro-N-{5-[4-methyl-1-(5-methyl-thiazol-2-yl)-1,2,5,6-tetrahydro-pyridin-3-yl]-pyrazin-2-yl}-benzamid (R02959),
2,6-Difluoro-N-(1-(4-hydroxy-2-(trifluoromethyl)benzyl)-1H-pyrazol-3-yl)benzamide (GSK-7975A),
2,6-Difluoro-N-(1-(2-phenoxybenzyl)-1H-pyrazol-3-yl)benzamide (GSK5503A),
N-(2′,5′-Dimethoxy[1,1′-biphenyl]-4-yl)-3-fluoro-4-pyridinecarboxamide (Synta 66),
or a pharmaceutically acceptable salt thereof.

54. The kit according to any one of claims 49-53, wherein the CRAC modulator is a compound of formula (IA) 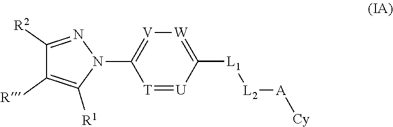
or a tautomer, N-oxide, pharmaceutically acceptable ester, or pharmaceutically acceptable salt thereof, wherein
both R¹ and R² are cyclopropyl or one of R¹ and R² is CF₃ and the other is cyclopropyl;
T is CF or N and U, V, W are independently CH, CF or N;
L₁ and L₂ together represent —NH—C(═X)—, —NH—S(═O)_{q}—, —C(═X)NH—, or —S(═O)_{q}NH— or —NH—CR′R″—;
A is absent or selected from —(CR′R″)— and —NR^{a};
each occurrence of R′ and R″ are the same or different and are independently selected from hydrogen or substituted or unsubstituted C₍₁₋₆₎ alkyl group or R′ and R″ may be joined to form a substituted or unsubstituted saturated or unsaturated 3-6 membered ring, which may optionally include one or more heteroatoms which may be same or different and are selected from O, NR^{a} and S;
R′″ is selected from hydrogen or halogen;
each occurrence of X is independently selected from O, S and —NR^{a};
Cy is selected from 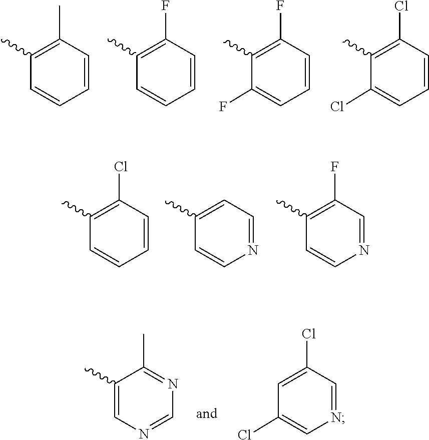
each occurrence of R^{a} is independently selected from hydrogen, nitro, hydroxy, cyano, halogen, —OR^{c}, —S(═O)_{q}—R^{c}, —NR^{c}R^{d}, —C(═Y)—R^{c}, —CR^{c}R^{d}—C(═Y)—R^{c}, —CR^{c}R^{d}—Y—CR^{c}R^{d}—, —C(═Y)—NR^{c}R^{d}—, —NRR^{d}—C(═Y)—NR^{c}R^{d}—, —S(═O)_{q}—NR^{c}R^{d}—, —NR^{c}R^{d}—S(═O)_{q}—NR^{c}R^{d}—, —NR^{c}R^{d}—NR^{c}R^{d}—, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted heterocylyl, substituted or unsubstituted heterocyclylalkyl, substituted or unsubstituted aryl, substituted or unsubstituted arylalkyl, substituted or unsubstituted heteroaryl, and substituted or unsubstituted heteroarylalkyl;
each occurrence of R^{c} and R^{d} may be same or different and are independently selected from hydrogen, nitro, hydroxy, cyano, halogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted heterocyclic group, substituted or unsubstituted heterocyclylalkyl, or when two R^{c} and/or R^{d} substitutents are directly bound to the same atom, they may be joined to form a substituted or unsubstituted saturated or unsaturated 3-10 member ring, which may optionally include one or more heteroatoms which are the same or different and are selected from O, NH and S;
each occurrence of Y is independently selected from O, S and —NR^{a}; and
each occurrence of q independently represents 0, 1 or 2.

55. The kit according to any one of claims 49-54, wherein the CRAC modulator is a compound of formula (IB) 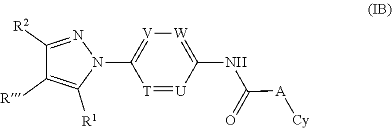
or a tautomer, N-oxide, pharmaceutically acceptable ester or pharmaceutically acceptable salt thereof, wherein
R¹ and R² are both cyclopropyl or one of R¹ and R² is CF₃ and the other is cyclopropyl;
R′″ is selected from hydrogen, hydroxy, cyano, halogen, —OR^{a}, —COOR^{a}, —S(═O)_{q}—R^{a}, —NR^{a}R^{b}, —C(═X)—R^{a}, substituted or unsubstituted C₍₁₋₆₎ alkyl group, substituted or unsubstituted C₍₁₋₆₎ alkenyl, substituted or unsubstituted C₍₁₋₆₎ alkynyl, and substituted or unsubstituted C₍₃₋₅₎cycloalkyl;
T, U, V and W are the same or different and are independently selected from CR^{a} and N;
A is absent or is selected from —CH₂—, —CHMe-, 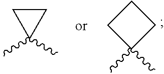
Cy is a bicyclic ring selected from substituted or unsubstituted cycloalkyl group, substituted or unsubstituted heterocyclyl, substituted or unsubstituted aryl, and substituted or unsubstituted heteroaryl;
each occurrence of R^{a} and R^{b} are the same or different and are independently selected from hydrogen, nitro, hydroxy, cyano, halogen, —OR^{c}, —S(═O)_{q}—R^{c}, —NR^{c}R^{d}, —C(═Y)—R^{c}, —CR^{c}R^{d}—C(═Y)—R^{c}, —CR^{c}R^{d}—Y—CR^{c}R^{d}—, —C(═Y)—NR^{c}R^{d}—, —NRR^{d}—C(═Y)—NR^{c}R^{d}—, —S(═O)_{q}—NR^{c}R^{d}—, —NR^{c}R^{d}—S(═O)_{q}—NR^{c}R^{d}—, —NR^{c}R^{d}—NR^{c}R^{d}—, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted heterocylyl, substituted or unsubstituted heterocyclylalkyl, substituted or unsubstituted aryl, substituted or unsubstituted arylalkyl, substituted or unsubstituted heteroaryl, and substituted or unsubstituted heteroarylalkyl, or when R^{a} and R^{b} are directly bound to the same atom, they may be joined to form a substituted or unsubstituted saturated or unsaturated 3-10 member ring, which may optionally include one or more heteroatoms which may be the same or different and are selected from O, NR^{c} and S;
each occurrence of R^{c} and R^{d} may be same or different and are independently selected from hydrogen, nitro, hydroxy, cyano, halogen, substituted or unsubstituted alkyl, substituted or unsubstituted alkenyl, substituted or unsubstituted alkynyl, substituted or unsubstituted cycloalkyl, substituted or unsubstituted cycloalkylakyl, substituted or unsubstituted cycloalkenyl, substituted or unsubstituted heterocyclic group, substituted or unsubstituted heterocyclylalkyl, or when two R^{c} and/or R^{d} substitutents are directly bound to the same atom, they may be joined to form a substituted or unsubstituted saturated or unsaturated 3-10 member ring, which may optionally include one or more heteroatoms which are the same or different and are selected from O, NH and S;
each occurrence of Y is selected from O, S and —NR^{a}; and
each occurrence of q independently represents 0, 1 or 2.

56. The kit according to any one of claims 49-55, the CRAC modulator is selected from
N-[4-(3,5-dicyclopropyl-1H-pyrazol-1-yl)phenyl]-4-methyl-1,2,3-thiadiazole-5-carboxamide
N-[4-(3,5-dicyclopropyl-1H-pyrazol-1-yl)phenyl]-4-methylthiazole-5-carboxamide
N-[4-(3,5-dicyclopropyl-1H-pyrazol-1-yl)phenyl]-2,4-dimethylthiazole-5-carboxamide
N-[4-(3,5-dicyclopropyl-1H-pyrazol-1-yl)phenyl]-5-methylisoxazole-4-carboxamide
N-[4-(3,5-dicyclopropyl-1H-pyrazol-1-yl)phenyl]-3,5-dimethylisoxazole-4-carboxamide
N-[4-(3,5-dicyclopropyl-1H-pyrazol-1-yl)phenyl]benzamide
N-[4-(3,5-dicyclopropyl-1H-pyrazol-1-yl)phenyl]-2-methylbenzamide
N-[4-(3,5-dicyclopropyl-1H-pyrazol-1-yl)phenyl]-2,6-difluorobenzamide
N-[4-(3,5-dicyclopropyl-1H-pyrazol-1-yl)phenyl]-2,3-difluorobenzamide
N-[4-(3,5-dicyclopropyl-1H-pyrazol-1-yl)phenyl-3-(methylsulfonyl)benzamide
N-[4-(3,5-dicyclopropyl-1H-pyrazol-1-yl)phenyl]-4-(methylsulfonyl)benzamide
2-chloro-N-[4-(3,5-dicyclopropyl-1H-pyrazol-1-yl)phenyl]-5-(methylthio)benzamide
2-chloro-N-[4-(3,5-dicyclopropyl-1H-pyrazol-1-yl)phenyl)-5-(methylsulfonyl)benzamide
N-[4-(3,5-dicyclopropyl-1H-pyrazol-1-yl)phenyl]nicotinamide hydrochloride
N-[4-(3,5-dicyclopropyl-1H-pyrazol-1-yl)phenyl]isonicotinamide hydrochloride
N-[4-(3,5-dicyclopropyl-1H-pyrazol-1-yl)phenyl]-3-fluoroisonicotinamide
3,5-dichloro-N-(4-(3,5-dicyclopropyl-1H-pyrazol-1-yl)phenyl)isonicotinamide
N-[4-(3,5-dicyclopropyl-1H-pyrazol-1-yl)phenyl]-4-methylpyrimidine-5-carboxamide
N-[4-(3,5-dicyclopropyl-1H-pyrazol-1-yl)phenyl]-2-phenylacetamide
N-[4-(3,5-dicyclopropyl-1H-pyrazol-1-yl)phenyl]-2-(4-fluorophenyl)acetamide
N-[4-(3,5-dicyclopropyl-1H-pyrazol-1-yl)phenyl]-1-phenylcyclopropanecarboxamide
N-[4-(3,5-dicyclopropyl-1H-pyrazol-1-yl)phenyl]-2-(pyridin-2-yl)acetamide
N-[4-(3,5-dicyclopropyl-1H-pyrazol-1-yl)phenyl]-2-(pyridin-3-yl)acetamide
N-[4-(3,5-dicyclopropyl-1H-pyrazol-1-yl)phenyl]-2-(pyridin-4-yl)acetamide
N-[4-(3,5-dicyclopropyl-1H-pyrazol-1-yl)phenyl]-2-(piperazin-1-yl)acetamide
N-[4-(3,5-dicyclopropyl-1H-pyrazol-1-yl)phenyl]-2-morpholinoacetamide
N-[4-(3,5-dicyclopropyl-1H-pyrazol-1-yl)phenyl]benzenesulfonamide
N-[4-(3,5-dicyclopropyl-1H-pyrazol-1-yl)-3-fluorophenyl]-4-methyl-1,2,3-thiadiazole-5-carboxamide
N-[4-(3,5-dicyclopropyl-1H-pyrazol-1-yl)-3-fluorophenyl]-4-methylthiazole-5-carboxamide
N-[4-(3,5-dicyclopropyl-1H-pyrazol-1-yl)-3-fluorophenyl]-3,5-dimethylisoxazole-4-carboxamide
N-[4-(3,5-dicyclopropyl-1H-pyrazol-1-yl)-3-fluorophenyl]-2methyl benzamide
N-[4-(3,5-dicyclopropyl-1H-pyrazol-1-yl)-3-fluorophenyl]-2,3-difluorobenzamide
N-[4-(3,5-dicyclopropyl-1H-pyrazol-1-yl)-3-fluorophenyl]-2,6-difluorobenzamide
N-[4-(3,5-dicyclopropyl-1H-pyrazol-1-yl)-3-fluorophenyl]nicotinamide
N-[4-(3,5-dicyclopropyl-1H-pyrazol-1-yl)-3-fluorophenyl]isonicotinamide
N-[4-(3,5-dicyclopropyl-1H-pyrazol-1-yl)-3-fluorophenyl]-4-methylpyrimidine-5-carboxamide
N-[4-(4-chloro-3,5-dicyclopropyl-1H-pyrazol-1-yl)-3-fluorophenyl]-4-methyl-1,2,3-thiadiazole-5-carboxamide
N-[6-(3,5-dicyclopropyl-1H-pyrazol-1-yl)pyridin-3-yl]-4-methyl-1,2,3-thiadiazole-5-carboxamide
N-[6-(3,5-dicyclopropyl-1H-pyrazol-1-yl)pyridin-3-yl]-4-methylthiazole-5-carboxamide
N-[6-(3,5-dicyclopropyl-1H-pyrazol-1-yl)pyridin-3-yl]-2,4-dimethylthiazole-5-carboxamide
N-[6-(3,5-dicyclopropyl-1H-pyrazol-1-yl)pyridin-3-yl]-3,5-dimethylisoxazole-4-carboxamide
6-(3,5-dicyclopropyl-1H-pyrazol-1-yl)-N-o-tolylnicotinamide
N-[6-(3,5-dicyclopropyl-1H-pyrazol-1-yl)pyridin-3-yl]-2-fluorobenzamide
N-[6-(3,5-dicyclopropyl-1H-pyrazol-1-yl)pyridin-3-yl]-2,3-difluorobenzamide
N-[6-(3,5-dicyclopropyl-1H-pyrazol-1-yl)pyridin-3-yl]-2,6-difluorobenzamide
N-[6-(3,5-dicyclopropyl-1H-pyrazol-1-yl)pyridin-3-yl]nicotinamide dihydrochloride
N-[6-(3,5-dicyclopropyl-1H-pyrazol-1-yl)pyridin-3-yl]isonicotinamide
N-[6-(3,5-dicyclopropyl-1H-pyrazol-1-yl)pyridin-3-yl]-3-fluoroisonicotinamide
3,5-dichloro-N-{4-[5-cyclopropyl-3-(trifluoromethyl)-1H-pyrazol-1-yl]phenyl}isonicotinamide
3,5-dichloro-N-[6-(3,5-dicyclopropyl-1H-pyrazol-1-yl)pyridin-3-yl]isonicotinamide
N-[6-(3,5-dicyclopropyl-1H-pyrazol-1-yl)pyridin-3-yl]-4-methylpyrimidine-5-carboxamide
N-{4-[5-cyclopropyl-3-(trifluoromethyl)-1H-pyrazol-1-yl]phenyl}-4-methyl-1,2,3-thiadiazole-5-carboxamide
N-{4-[5-cyclopropyl-3-(trifluoromethyl)-1H-pyrazol-1-yl]phenyl}-4-methylthiazole-5-carboxamide
N-{4-[5-cyclopropyl-3-(trifluoromethyl)-1H-pyrazol-1-yl]phenyl}-N,4-dimethylthiazole-5-carboxamide
N-{4-[5-cyclopropyl-3-(trifluoromethyl)-1H-pyrazol-1-yl]phenyl}-2,4-dimethylthiazole-5-carboxamide
N-{4-[5-cyclopropyl-3-(trifluoromethyl)-1H-pyrazol-1-yl]phenyl}-5-methylisoxazole-4-carboxamide
N-{4-[5-cyclopropyl-3-(trifluoromethyl)-1H-pyrazol-1-yl]phenyl}-3,5-dimethylisoxazole-4-carboxamide
N-{4-[5-cyclopropyl-3-(trifluoromethyl)-1H-pyrazol-1-yl]phenyl}-1-methyl-1H-imidazole-2-carboxamide
N-{4-[3-cyclopropyl-5-(trifluoromethyl)-1H-pyrazol-1-yl]phenyl}-4-methyl-1H-imidazole-5-carboxamide
N-{4-[5-cyclopropyl-3-(trifluoromethyl)-1H-pyrazol-1-yl]phenyl}-2-methylbenzamide
N-{4-[5-cyclopropyl-3-(trifluoromethyl)-1H-pyrazol-1-yl]phenyl}-2,3-difluorobenzamide
N-{4-[5-cyclopropyl-3-(trifluoromethyl)-1H-pyrazol-1-yl]phenyl}-2,6-difluorobenzamide
N-{4-[5-cyclopropyl-3-(trifluoromethyl)-1H-pyrazol-1-yl]phenyl}-3-(methylsulfonyl) benzamide
2-chloro-N-{4-[5-cyclopropyl-3-(trifluoromethyl)-1H-pyrazol-1-yl]phenyl}-5-(methylthio) benzamide
2-chloro-N-{4-[5-cyclopropyl-3-(trifluoromethyl)-1H-pyrazol-1-yl]phenyl}-5-(methylsulfonyl)benzamide
N-{4-[5-cyclopropyl-3-(trifluoromethyl)-1H-pyrazol-1-yl]phenyl}pyridine-4-carboxamide hydrochloride
N-{4-[5-cyclopropyl-3-(trifluoromethyl)-1H-pyrazol-1-yl]phenyl}-3-fluoro isonicotinamide
N-{4-[5-cyclopropyl-3-(trifluoromethyl)-1H-pyrazol-1-yl]phenyl}-4-methylpyrimidine-5-carboxamide
N-{4-[5-cyclopropyl-3-(trifluoromethyl)-1H-pyrazol-1-yl]phenyl}-2,4-dimethyl pyrimidine-5-carboxamide
N-{4-[5-cyclopropyl-3-(trifluoromethyl)-1H-pyrazol-1-yl]phenyl}-2-(4-fluorophenyl)acetamide
N-{4-[5-cyclopropyl-3-(trifluoromethyl)-1H-pyrazol-1-yl]phenyl}-2-(pyridin-2-yl)acetamide
N-{4-[5-cyclopropyl-3-(trifluoromethyl)-1H-pyrazol-1-yl]phenyl}-2-(pyridin-3-yl)acetamide
N-{4-[5-cyclopropyl-3-(trifluoromethyl)-1H-pyrazol-1-yl]phenyl}-2-(pyridin-4-yl)acetamide
4-[5-cyclopropyl-3-(trifluoromethyl)-1H-pyrazol-1-yl]-N-[(4-methylthiazol-5-yl)methyl]aniline
1-{4-[5-cyclopropyl-3-(trifluoromethyl)-1H-pyrazol-1-yl]phenyl}-3-(4-methyl-1,2,3-thiadiazol-5-yl)urea
1-{4-[5-cyclopropyl-3-(trifluoromethyl)-1H-pyrazol-1-yl]phenyl}-3-(4-methylthiazol-5-yl)urea
1-{4-[5-cyclopropyl-3-(trifluoromethyl)-1H-pyrazol-1-yl]phenyl}-3-(4-methylpyrimidin-5-yl)urea
4-[5-cyclopropyl-3-(trifluoromethyl)-1H-pyrazol-1-yl]-N-(4-methylthiazol-5-yl) benzamide
4-[5-cyclopropyl-3-(trifluoromethyl)-1H-pyrazol-1-yl]-N-(2,6-difluorophenyl) benzamide
N-{4-[4-chloro-5-cyclopropyl-3-(trifluoromethyl)-1H-pyrazol-1-yl]phenyl}-4-methylthiazole-5-carboxamide
N-{4-[4-chloro-5-cyclopropyl-3-(trifluoromethyl)-1H-pyrazol-1-yl]phenyl}-2-(pyridin-2-yl)acetamide
N-{4-[5-cyclopropyl-3-(trifluoromethyl)-1H-pyrazol-1-yl]-3-fluorophenyl}-4-methyl-1,2,3-thiadiazole-5-carboxamide
N-{4-[5-cyclopropyl-3-(trifluoromethyl)-1H-pyrazol-1-yl]-3-fluorophenyl}-4-methylthiazole-5-carboxamide
N-{4-[5-cyclopropyl-3-(trifluoromethyl)-1H-pyrazol-1-yl]-3-fluorophenyl}-5-methylisoxazole-4-carboxamide
N-{4-[5-cyclopropyl-3-(trifluoromethyl)-1H-pyrazol-1-yl]-3-fluorophenyl}-3,5-dimethylisoxazole-4-carboxamide
N-{4-[5-cyclopropyl-3-(trifluoromethyl)-1H-pyrazol-1-yl]-3-fluorophenyl}-2-methylbenzamide
N-{4-[5-cyclopropyl-3-(trifluoromethyl)-1H-pyrazol-1-yl]-3-fluorophenyl}-2,3-difluorobenzamide
N-{4-[5-cyclopropyl-3-(trifluoromethyl)-1H-pyrazol-1-yl]-3-fluorophenyl}-2,6-difluorobenzamide
N-{4-[5-cyclopropyl-3-(trifluoromethyl)-1H-pyrazol-1-yl]-3-fluorophenyl}nicotinamide
N-{4-[5-cyclopropyl-3-(trifluoromethyl)-1H-pyrazol-1-yl]-3-fluorophenyl}isonicotinamide
N-{4-[5-cyclopropyl-3-(trifluoromethyl)-1H-pyrazol-1-yl]-3-fluorophenyl}-3-fluoroisonicotinamide
3,5-dichloro-N-{4-[5-cyclopropyl-3-(trifluoromethyl)-1H-pyrazol-1-yl]-3-fluorophenyl}isonicotinamide
N-{4-[5-cyclopropyl-3-(trifluoromethyl)-1H-pyrazol-1-yl]-3-fluorophenyl}-4-methylpyrimidine-5-carboxamide
N-{4-[5-cyclopropyl-3-(trifluoromethyl)-1H-pyrazol-1-yl]-3-fluorophenyl}-N,4-dimethylpyrimidine-5-carboxamide
N-{4-[4-chloro-5-cyclopropyl-3-(trifluoromethyl)-1H-pyrazol-1-yl]-3-fluorophenyl}-4-methyl-1,2,3-thiadiazole-5-carboxamide
N-{4-[4-chloro-5-cyclopropyl-3-(trifluoromethyl)-1H-pyrazol-1-yl]-3-fluorophenyl}-2-(pyridin-2-yl)acetamide
1-{4-[5-cyclopropyl-3-(trifluoromethyl)-1H-pyrazol-1-yl]-3-fluorophenyl}-3-(4-methylpyrimidin-5-yl)urea
N-{4-[5)-cyclopropyl-3-(trifluromethyl)-1H-pyrazol-1-yl]3-fluorophenyl}-2,6-dichloro benzamide
4-[5-cyclopropyl-3-(trifluoromethyl)-1H-pyrazol-1-yl]-N-(2,3-difluorophenyl)-3-fluorobenzamide
4-[5-cyclopropyl-3-(trifluoromethyl)-1H-pyrazol-1-yl]-N-(2,6-difluorophenyl)-3-fluorobenzamide
N-{6-[5-cyclopropyl-3-(trifluoromethyl)-1H-pyrazol-1-yl]pyridin-3-yl}-4-methyl-1,2,3-thiadiazole-5-carboxamide
N-{6-[5-cyclopropyl-3-(trifluoromethyl)-1H-pyrazol-1-yl]pyridin-3-yl}-4-methylthiazole-5-carboxamide
N-{6-[5-cyclopropyl-3-(trifluoromethyl)-1H-pyrazol-1-yl]pyridin-3-yl}-3,5-dimethylisoxazole-4-carboxamide
N-{6-[5-cyclopropyl-3-(trifluoromethyl)-1H-pyrazol-1-yl]pyridin-3-yl}-2-methylbenzamide
2-chloro-N-{6-[5-cyclopropyl-3-(trifluoromethyl)-1H-pyrazol-1-yl]pyridin-3-yl}benzamide
N-(6-(5-cyclopropyl-3-(trifluoromethyl)-1H-pyrazol-1-yl)pyridin-3-yl)-2-fluorobenzamide
N-{6-[5-cyclopropyl-3-(trifluoromethyl)-1H-pyrazol-1-yl]pyridin-3-yl}-2,3-difluorobenzamide
N-{6-[5-cyclopropyl-3-(trifluoromethyl)-1H-pyrazol-1-yl]pyridin-3-yl}-2,6-difluorobenzamide
N-{6-[5-cyclopropyl-3-(trifluoromethyl)-1H-pyrazol-1-yl]pyridin-3-yl}picolinamide
N-{6-[5-cyclopropyl-3-(trifluoromethyl)-1H-pyrazol-1-yl]pyridin-3-yl}-3-methylpicolinamide
N-{6-[5-cyclopropyl-3-(trifluoromethyl)-1H-pyrazol-1-yl]pyridin-3-yl}nicotinamide
N-{6-[5-cyclopropyl-3-(trifluoromethyl)-1H-pyrazol-1-yl]pyridin-3-yl}-2-methylnicotinamide
N-{6-[5-cyclopropyl-3-(trifluoromethyl)-1H-pyrazol-1-yl]pyridin-3-yl}isonicotinamide
N-{6-[5-cyclopropyl-3-(trifluoromethyl)-1H-pyrazol-1-yl]pyridin-3-yl}-3-fluoroisonicotinamide
3,5-dichloro-N-{6-[5-cyclopropyl-3-(trifluoromethyl)-1H-pyrazol-1-yl]pyridin-3-yl}isonicotinamide
N-{6-[5-cyclopropyl-3-(trifluoromethyl)-1H-pyrazol-1-yl]pyridin-3-yl}-4-methylpyrimidine-5-carboxamide
N-{6-[5-cyclopropyl-3-(trifluoromethyl)-1H-pyrazol-1-yl]pyridin-3-yl}-2-(pyridin-2-yl)acetamide
N-{6-[5-cyclopropyl-3-(trifluoromethyl)-1H-pyrazol-1-yl]pyridin-3-yl}-2-(pyridin-4-yl)acetamide
N-{4-[4-chloro-5-cyclopropyl-3-(trifluoromethyl)-1H-pyrazol-1-yl]-3-fluorophenyl}-4-methylpyrimidine-5-carboxamide
1-{6-[3-cyclopropyl-5-(trifluoromethyl)-1H-pyrazol-1-yl]pyridin-3-yl}-3-(4-methylthiazol-5-yl)urea
6-[5-cyclopropyl-3-(trifluoromethyl)-1H-pyrazol-1-yl]-N-(2,3-difluorophenyl) nicotinamide
6-[5-cyclopropyl-3-(trifluoromethyl)-1H-pyrazol-1-yl]-N-(2,6-difluorophenyl) nicotinamide
N-{6-[4-chloro-5-cyclopropyl-3-(trifluoromethyl)-1H-pyrazol-1-yl]pyridin-3-yl}-4-methylthiazole-5-carboxamide
N-{2-[5-cyclopropyl-3-(trifluoromethyl)-1H-pyrazol-1-yl]pyrimidin-5-yl}-2,6-difluorobenzamide
N-{4-[5-(fluoromethyl)-3-(trifluoromethyl)-1H-pyrazol-1-yl]phenyl}-4-methylthiazole-5-carboxamide
N-{4-[5-(difluoromethyl)-3-(trifluoromethyl)-1H-pyrazol-1-yl]phenyl}-4-methylthiazole-5-carboxamide
3,5-dichloro-N-[4-(3,5-dicyclopropyl-1H-pyrazol-1-yl)-3-fluorophenyl]isonicotinamide
N-(2-chloro-6-fluorophenyl)-4-[5-cyclopropyl-3-(trifluoromethyl)-1H-pyrazol-1-yl]-3-fluorobenzamide
N-{2-[5-cyclopropyl-3-(trifluoromethyl)-1H-pyrazol-1-yl]pyrimidin-5-yl}-4-methylthiazole-5-carboxamide
N-{4-[5-cyclopropyl-3-(trifluoromethyl)-1H-pyrazol-1-yl]-3,5-difluorophenyl}-4-methylpyrimidine-5-carboxamide
{4-[5-cyclopropyl-3-(trifluoromethyl)-1H-pyrazol-1-yl]-3-fluorophenyl}-1-phenylcyclobutanecarboxamide
N-{4-[5-cyclopropyl-3-(trifluoromethyl)-1H-pyrazol-1-yl]-3-fluorophenyl}-4-methyloxazole-5-carboxamide
N-{2-[5-cyclopropyl-3-(trifluoromethyl)-1H-pyrazol-1-yl]pyrimidin-5-yl}-4-methylpyrimidine-5-carboxamide
4-[5-cyclopropyl-3-(trifluoromethyl)-1H-pyrazol-1-yl]-3-fluoro-N-(4-methylpyrimidin-5-yl) benzamide and
N-{4-[3-cyclopropyl-5-(difluoromethyl)-1H-pyrazol-1-yl]-3-fluorophenyl}-2,6-difluorobenzamide;
N-{4-[5-cyclopropyl-3-(difluoromethyl)-1H-pyrazol-1-yl]-3-fluorophenyl}-2,6-difluorobenzamide
N-[4-(3,5-dicyclopropyl-1H-pyrazol-1-yl)phenyl]-1H-benzo[d]imidazole-6-carboxamide
N-[4-(3,5-dicyclopropyl-1H-pyrazol-1-yl)phenyl]-1H-benzo[d][1,2,3]triazole-6-carboxamide
N-[4-(3,5-dicyclopropyl-1H-pyrazol-1-yl)phenyl]quinoline-6-carboxamide hydrochloride
N-[4-(3,5-dicyclopropyl-1H-pyrazol-1-yl)phenyl]quinoxaline-6-carboxamide
2-(1H-benzo[d]imidazol-1-yl)-N-[4-(3,5-dicyclopropyl-1H-pyrazol-1-yl)phenyl]acetamide
2-(1H-benzo[d][1,2,3]triazol-1-yl)-N-[4-(3,5-dicyclopropyl-1H-pyrazol-1-yl)phenyl]acetamide
N-[4-(3,5-dicyclopropyl-1H-pyrazol-1-yl)phenyl]-2-(1H-indol-3-yl)acetamide
N-[4-(3,5-dicyclopropyl-1H-pyrazol-1-yl)phenyl]-2-(imidazo[1,2-a]pyridin-2-yl)acetamide hydrochloride
N-[4-(3,5-dicyclopropyl-1H-pyrazol-1-yl)phenyl]-2-(quinolin-6-yl)acetamide:
N-[4-(3,5-dicyclopropyl-1H-pyrazol-1-yl)phenyl]-2-(quinolin-6-yl)acetamide hydrochloride
2-(1H-benzo[d][1,2,3]triazol-1-yl)-N-(4-(3,5-dicyclopropyl-1H-pyrazol-1-yl)-3-fluorophenyl)acetamide
N-[4-(3,5-dicyclopropyl-1H-pyrazol-1-yl)-3-fluorophenyl]-2-(quinolin-6-yl)acetamide hydrochloride
N-[6-(3,5-dicyclopropyl-1H-pyrazol-1-yl)pyridin-3-yl]quinoline-6-carboxamide dihydrochloride
N-[6-(3,5-dicyclopropyl-1H-pyrazol-1-yl)pyridin-3-yl]quinoxaline-6-carboxamide
2-(1H-benzo[d][1,2,3]triazol-1-yl)-N-[6-(3,5-dicyclopropyl-1H-pyrazol-1-yl)pyridin-3-yl]acetamide
N-[6-(3,5-dicyclopropyl-1H-pyrazol-1-yl)pyridin-3-yl]-2-(quinolin-6-yl)acetamidedihydrochloride
N-{4-[5-cyclopropyl-3-(trifluoromethyl)-1H-pyrazol-1-yl]phenyl}quinoline-6-carboxamide hydrochloride
N-{4-[5-cyclopropyl-3-(trifluoromethyl)-1H-pyrazol-1-yl]phenyl}quinoxaline-6-carboxamide
2-(1H-benzo[d]imidazol-1-yl)-N-{4-[5-cyclopropyl-3-(trifluoromethyl)-1H-pyrazol-1-yl]phenyl}acetamide
2-(1H-benzo[d][1,2,3]triazol-1-yl)-N-{4-[5-cyclopropyl-3-(trifluoromethyl)-1H-pyrazol-1-yl]phenyl}acetamide
2-(2H-benzo[d][1,2,3]triazol-2-yl)-N-{4-[5-cyclopropyl-3-(trifluoromethyl)-1H-pyrazol-1-yl]phenyl}acetamide
2-(3H-[1,2,3]triazolo[4,5-b]pyridin-3-yl)-N-{4-[5-cyclopropyl-3-(trifluoromethyl)-1H-pyrazol-1-yl]phenyl}acetamide
(S)-2-(3H-[1,2,3]triazolo[4,5-b]pyridin-3-yl)-N-{4-[5-cyclopropyl-3-(trifluoromethyl)-1H-pyrazol-1-yl]phenyl}propanamide
2-(6-amino-9H-purin-9-yl)-N-{4-[5-cyclopropyl-3-(trifluoromethyl)-1H-pyrazol-1-yl]phenyl}acetamide
N-(4-(5-cyclopropyl-3-(trifluoromethyl)-1H-pyrazol-1-yl)phenyl)-2-(1,3-dimethyl-2,6-dioxo-2,3-dihydro-1H-purin-7(6H)-yl)acetamide
N-{4-[5-cyclopropyl-3-(trifluoromethyl)-1H-pyrazol-1-yl)phenyl)-2-(imidazo[1,2-a]pyridin-2-yl)acetamide hydrochloride
N-{4-[5-cyclopropyl-3-(trifluoromethyl)-1H-pyrazol-1-yl]phenyl}-2-(quinolin-6-yl)acetamide hydrochloride
N-{4-[5-cyclopropyl-3-(trifluoromethyl)-1H-pyrazol-1-yl]phenyl}-2-(quinolin-6-yl)propanamide hydrochloride
N-{4-[5-cyclopropyl-3-(trifluoromethyl)-1H-pyrazol-1-yl]-3-fluorophenyl}-1H-benzo[d][1,2,3]triazole-6-carboxamide
2-(1H-benzo[d][1,2,3]triazol-1-yl)-N-{4-[5-cyclopropyl-3-(trifluoromethyl)-1H-pyrazol-1-yl]-3-fluorophenyl}acetamide
N-{6-[5-cyclopropyl-3-(trifluoromethyl)-1H-pyrazol-1-yl]pyridin-3-yl}-1H-benzo[d][1,2,3]triazole-5-carboxamide
2-(1H-benzo[d][1,2,3]triazol-1-yl)-N-{6-[5-cyclopropyl-3-(trifluoromethyl)-1H-pyrazol-1-yl]pyridin-3-yl}acetamide
2-(2H-benzo[d][1,2,3]triazol-2-yl)-N-{6-[5-cyclopropyl-3-(trifluoromethyl)-1H-pyrazol-1-yl]pyridin-3-yl}acetamide
N-{6-[5-cyclopropyl-3-(trifluoromethyl)-1H-pyrazol-1-yl]pyridin-3-yl}-2-(quinolin-6-yl)acetamide hydrochloride
2-(1H-benzo[d][1,2,3]triazol-1-yl)-N-{6-[4-chloro-5-cyclopropyl-3-(trifluoromethyl)-1H-pyrazol-1-yl]pyridin-3-yl}acetamide
4-[5-cyclopropyl-3-(trifluoromethyl)-1H-pyrazol-1-yl]-3-fluoro-N-(quinolin-6-ylmethyl) benzamide hydrochloride
1-[4-(3,5-dicyclopropyl-1H-pyrazol-1-yl)phenyl]-3-(quinolin-6-yl)urea;
and pharmaceutically acceptable salts thereof.

57. The kit according to any one of claims 49-56, wherein the CRAC modulator is selected from
CM2489;
CM4620;
N-(5-(6-chloro-2,2-difluorobenzo[d][1,3]dioxol-5-yl)pyrazin-2-yl)-2-fluoro-6-methylbenzamide;
N-[4-[3,5-Bis(trifluoromethyl)-1H-pyrazol-1-yl]phenyl]-4-methyl-1,2,3-thiadiazole-5-carboxamide (YM-58483);
2,6-Difluoro-N-{5-[4-methyl-1-(5-methyl-thiazol-2-yl)-1,2,5,6-tetrahydro-pyridin-3-yl]-pyrazin-2-yl}-benzamid (R02959);
2,6-Difluoro-N-(1-(4-hydroxy-2-(trifluoromethyl)benzyl)-1H-pyrazol-3-yl)benzamide (GSK-7975A);
2,6-Difluoro-N-(1-(2-phenoxybenzyl)-1H-pyrazol-3-yl)benzamide (GSK5503A);
N-(2′,5′-Dimethoxy[1,1′-biphenyl]-4-yl)-3-fluoro-4-pyridinecarboxamide (Synta 66);
N-{6-[5-cyclopropyl-3-(trifluoromethyl)-1H-pyrazol-1-yl]pyridin-3-yl}-2-methylbenzamide;
N-{6-[5-cyclopropyl-3-(trifluoromethyl)-1H-pyrazol-1-yl]pyridin-3-yl}-3-fluoroisonicotinamide;
and pharmaceutically acceptable salts thereof

58. The kit according to any one of claims 49-57, wherein the CRAC modulator is selected from
N-{6-[5-cyclopropyl-3-(trifluoromethyl)-1H-pyrazol-1-yl]pyridin-3-yl}-2-methyl benzamide and
N-{6-[5-cyclopropyl-3-(trifluoromethyl)-1H-pyrazol-1-yl]pyridin-3-yl}-3-fluoroisonicotinamide and pharmaceutically acceptable salts thereof.

59. The kit according to any one of claims 49-58, wherein the CRAC modulator is N-{6-[5-cyclopropyl-3-(trifluoromethyl)-1H-pyrazol-1-yl]pyridin-3-yl}-2-methyl benzamide and the corticosteroid is dexamethasone.

60. The kit according to any one of claims 49-58, wherein the CRAC modulator is
N-{6-[5-cyclopropyl-3-(trifluoromethyl)-1H-pyrazol-1-yl]pyridin-3-yl}-2-methyl benzamide and the corticosteroid is fluticasone.

61. The kit according to any one of claims 49-58, wherein the CRAC modulator is
N-{6-[5-cyclopropyl-3-(trifluoromethyl)-1H-pyrazol-1-yl]pyridin-3-yl}-2-methyl benzamide and the corticosteroid is mometasone, mometasone furoate or mometasone furoate monohydrate.
